SEQUENCE LISTING (40 sequences)

SEQ ID NO: 1 - protein, 30 aa, Homo sapiens
GLYECNRYPCIKEVECYVSRPTEKTVFLVF

SEQ ID NO: 2 - protein, 30 aa, Mus musculus; identical to SEQ ID NO: 1

SEQ ID NO: 3 - protein, 30 aa, Macaca mulatta; identical to SEQ ID NO: 1

SEQ ID NO: 4 - protein, 30 aa, Callithrix jacchus; identical to SEQ ID NO: 1

SEQ ID NO: 5 - protein, 30 aa, Taeniopygia guttata
AIFECDRYPCVKEVECYVSRPTEKSVFLVF

SEQ ID NO: 6 - protein, 30 aa, Danio rerio
AVYECDRYPCIKDVECYVSRPTEKTVFLVF

SEQ ID NO: 7 - protein, 36 aa, Homo sapiens
LIQWYIYGFSLSAVYTCKRDPCPHQVDCFLSRPTEK

SEQ ID NO: 8 - protein, 36 aa, Mus musculus; identical to SEQ ID NO: 7

SEQ ID NO: 9 - protein, 36 aa, Macaca mulatta; identical to SEQ ID NO: 7

SEQ ID NO: 10 - protein, 36 aa, Callithrix jacchus; identical to SEQ ID NO: 7

SEQ ID NO: 11 - protein, 36 aa, Taeniopygia guttata
LIQWYIYGFSLNAIYTCERDPCPHRVDCFLSRPTEK

SEQ ID NO: 12 - protein, 36 aa, Danio rerio
VIQWYLYGFSLSAVYTCERTPCPHRVDCFLSRPTEK

SEQ ID NO: 13 - protein, 23 aa, Homo sapiens
GFQVHPFYVCSRLPCPHKIDCFI

SEQ ID NO: 14 - protein, 23 aa, Mus musculus
GFQVHPFYVCSRLPCPHKIDCFI

SEQ ID NO: 15 - protein, 23 aa, Macaca mulatta
GFQVHPFYVCSRLPCPHKIDCFI

SEQ ID NO: 16 - protein, 23 aa, Callithrix jacchus
GFQVHPFYVCSRLPCPHKIDCFI

SEQ ID NO: 17 - protein, 23 aa, Taeniopygia guttata
RFEVSPSYVCSRSPCPHTVDCFV

SEQ ID NO: 18 - protein, 23 aa, Danio rerio
GFEVAPSYVCTRSPCPHTVDCFV

SEQ ID NO: 19 - protein, 306 aa, Morone americana
MGEWTILERLLEAAVQQHSTMIGRILLTVVVIFRILIVAIVGETVYEDEQAMFICNTLQPGCNQACYDKAFPISHIRYWVFQIILVCTPSLCFITYSVHQSAKQKDRRYSFLYPIMERDYGGRDGARKLRNINGILVQHGGDGGGGKEEPDCLEVKEIPNAPRGLTHGKSSKVRRQEGISRFYIIQVVFRNALEIGFLAGQYFLYGFSVPGIFECDRYPCLKEVECYVSRPTEKTVFLVFMFAVSGICVVLNLAELNHLGWRKIKAAIRGVQARRKSICEIRKKDMAHLSQPPNLGRTQSSESAYV

SEQ ID NO: 20 - protein, 304 aa, Morone americana
MGEWTILERLLEAAVQQHSTMIGRILLTVVVIFRILIVAIVGETVYDDEQTMFVCNTLQPGCNQACYDKAFPISHIRYWVFQIIMVCTPSLCFITYSVHQSAKQKERRYSTVYLTLDKDQDSLKRDESKKIKNTIVNGVLQNTENSTKEAEPDCLEVKEIPNSAMRTTKSKMRRQEGISRFYIIQVVFRNALEIGFLVGQYFLYGFNVPSVYECDRYPCIKDVECYVSRPTEKTVFLVFMFAVSGFCVVLNLAELNHLGWRKIKTAVRGVQARRKSIYEIRNKDLPRMSVPNFGRTQSSDSAYV

SEQ ID NO: 21 - DNA, 1871 bp, Morone americana
  source 1..1871: mol_type = other DNA
caagaacgctcatctgagcaggtgcatacacaaagaattctatagtctgagaaacacgagggtgggtttgtgtgcagctagcgtatagacaagcttcagtccgaccatctcaggtgcagttggggacatgtgtgcctaaaaaaagaccaaaggcagatcacagtagcaacaataaggtgccactgagctcctttaaggttgcttttgactcctcatttacaccagacaagccacctgaagccactccctacctcatatcaacccaacaagtggacttgaagactcagagaaaagagacttttaacaccaacactggtgccctgctgactgggaagcccccccacctgaatttctgtacccagtggatgcttcctaactccaaccatgggagaatggaccatcctagagcgcctcctggaggctgctgtgcagcagcactctactatgatcggaaggatcctgcttacagtagtggtgatcttccgtatcctgattgtggcaatcgttggtgagaccgtgtacgaggacgagcaggccatgttcatctgcaacactctgcagccaggctgcaaccaggcctgctacgacaaggccttccccatttcccacatccgctactgggtcttccagatcatactggtgtgcacgcccagcctctgctttatcacctactctgtccaccagtcggccaagcagaaagaccggcgttactcctttctctatcccataatggagagggactacgggggaagggacggggctcgaaagctccgcaacattaatggaattttagttcaacatggtggtgatggcggaggagggaaggaagaaccagactgcctggaggtgaaggagatccccaacgccccgcggggcctcacccacgggaagagctccaaggttcgccggcaagaagggatctcccgcttttacatcattcaggtggtgttcagaaacgcactggagattggcttcttggcgggccaatacttcctttatggctttagtgtgcctgggattttcgagtgcgaccgctacccgtgtctgaaggaggtggagtgctatgtgtcccgccccacagaaaaaacggttttcctggtgttcatgtttgcggtcagtggcatctgcgtagttctcaacttggccgaactcaaccatctgggctggcgcaagatcaaggccgccatcaggggtgtgcaggcccgcaggaagtcgatctgtgagatcaggaagaaggacatggcgcatctgtcccagccacccaacctgggacgcacacagtccagcgaatcggcctatgtctgatgatgaagagcagaagaagaggaggggtgggatgaatgtgaaagagaaagaatgaattaattatgattttaatttaaaagagatggtgttgaggtcaaacaatatgaccctctctgcgaaacagaatgtaaaagaccaaactccggctcagtcaagaaagagtgaaggagatcattaaaaaaaatagagagggtaaaagaggacataaagttctgcaaagaacgtaatgaacatttggtctcgtggggggatattccaactgccattttatacttttgtcttttttaagtaattaccgtctcctctgctcctcatttcttttaaagtgaggaattaaatcctcaaggggcttgagaaggaaggactagaaatgagaagaagaggggaagggagaggagaacggatgagaggctgttttgtattcttaggggatattggaagatgacaatggtgtttgatgcatcccatggtgcttttcttctggattgtgtcaacctcaatgaaaaaattgaggacaggataggggatgggacgggggaggatgagcagcaacatttaaaaaaaaa

SEQ ID NO: 22 - DNA, 1320 bp, Morone americana
  source 1..1320: mol_type = other DNA
cctgatctgaagatcgaaaataggagggcttatagtaatcagcccttcagactcccatgacaaggaaaagtgtggcaacgtcgtgggagcgctcggcacacacagcaggacgctcactggcgctgcgcgctcagcacataaccaaccagatagcgcgcacagagacggagagagacggggacggcagcagcatcccgtgacggatgcattttgtagctaaaatcggacgctttggagactgagaacaacgagaggcagggagaagaaggacggggaaataacgcgcttttttggagggattgaacagagtgccgttgtgctctagtcttctccggatgctcagaaatgggggaatggactatactagagaggctcctggaggctgctgtccagcagcactctactatgataggaaggatcctactaacagtggtggtcatcttccggattctaatcgtagcaatagttggagagactgtctatgatgacgagcagaccatgtttgtttgtaacaccttgcaaccgggctgcaaccaggcatgctacgacaaggcatttcccatttcacacataagatattgggtttttcagatcatcatggtgtgcaccccgagtctttgttttataacgtactcggtgcaccagtcggccaagcagaaggagcggcggtactcaacagtctatctgacactggataaggatcaagattcactgaagcgagacgagagcaaaaagataaagaacaccattgttaatggagtacttcagaacacggagaactccaccaaagaagccgagccagactgtttagaagtcaaagagatccctaattcggccatgagaactacaaagtccaaaatgaggcgacaagagggcatctccaggttttacatcatccaggtggttttcagaaacgcgctggaaataggcttcttggtgggtcagtatttcttgtatggattcaacgtcccgtcggtgtacgagtgcgatcgctacccctgcatcaaagatgtcgagtgctacgtctccagacccacggagaagacagtgttcctggtcttcatgttcgcggtcagcgggttctgcgtggtgctgaacctggcagaactcaatcacctgggctggaggaaaatcaaaacggccgtgagaggagtgcaggctcggcggaagtccatttatgagatcagaaataaggacttgccgaggatgagtgtgcctaatttcggccgtactcagtccagtgactctgcttatgtgtaacaaacacacacatacacacacacaaacggggaggaagaagtgaaaatatgtaaaaaaaaa

SEQ ID NO: 23 - DNA, 918 bp, synthetic construct
  source 1..918: mol_type = other DNA
atgggagaatggactatcctggaacgactcctggaagcggctgtgcagcagcactcaacgatgataggtcggattcttctgacagtggtcgtaatatttagaattctcattgtagccattgtgggagaaacagtatatgaagatgaacaagcaatgttcatctgtaacactctgcaacctggttgtaatcaagcttgctacgataaagcatttcccataagccatattagatattgggtttttcagataatccttgtctgtactccatctctttgcttcattacttactcagtgcatcaatctgccaagcagaaggaccgcagatacagcttcctttatcccataatggagcgcgattatggggggagagacggtgctcgaaaactccggaatataaatgggatcttggtgcaacatgggggggacggagggggaggtaaagaggagcccgactgtctcgaagtcaaagagatacctaatgctccccgcgggctcacccacggtaaatcctctaaggtaagaaggcaggagggaataagtcggttttacattatccaagtcgttttcaggaacgctctcgaaatcggatttcttgctggacagtatttcctgtacggtttttccgttcctggaatattcaagtgcgatagatacccttgcttgaaaaaagttgaatgctacgtgagccgacccacagagaagacagttttccttgtattcatgttcgccgtgtcagggatctgtgtcgtcctcaatttggccgaattgaaccatttgggctggcgcaagataaaggcggcaataagaggtgtccaggctcgaagaaagtcaatttgtgaaatcagaaaaaaagacatggcgcacctctcacagccgcctaatttgggacgaacacagtcaagcgagagtgcttatgtc

SEQ ID NO: 24 - DNA, 918 bp, synthetic construct
  source 1..918: mol_type = other DNA
atgggtgagtggactatacttgaacgcctgcttgaggctgctgtgcaacagcactcaacaatgattggacgcatcttgttgacagttgttgttatattccgcatcctgatcgtcgccattgtgggggaaactgtctacgaggatgagcaggctatgttcatttgcaatacattgcagcccggctgcaaccaggcttgttacgataaggcctttccaatctctcatatacggtactgggtattccaaattattcttgtttgtacaccctccctctgtttcataacctattctgtacaccagtccgctaaacagaaagatcgccgatacagcttcctgtaccccataatggaacgcgactatggaggtagagacggggcacgaaaactgcgcaacataaatggtattctggtgcagcatggcggtgatggagggggtggaaaggaagaaccagactgtttggaagtgaaggagattcccaatgcacctcgcggacttactcacggaaaatcaagcaaggtacgacgacaagaggggatcagcaggttctatatcattcaggtggtctttagaaatgctttggaaattggttttctggcaggccagtattttctttatggtttcagtgtacctggcatattcaagtgcgataggtatccatgcttgaaaaaagtagagtgttatgtgtctagacctaccgaaaagactgtcttccttgtctttatgttcgctgtttcaggtatctgcgtcgttctcaacctggctgagctcaaccatttgggctggagaaagataaaggcagcaatccgcggcgtgcaggcccggagaaagtctatctgtgagattaggaaaaaagatatggcccacctttcccagccccctaacctgggaaggacacaaagctcagaaagtgcttacgtc

SEQ ID NO: 25 - DNA, 912 bp, synthetic construct
  source 1..912: mol_type = other DNA
atgggtgagtggacaatccttgagaggctgctcgaagccgccgtccagcagcacagcaccatgataggtcgcatactgttgacagtagtagttatcttcagaatccttatcgtagctatcgttggggaaaccgtctatgacgatgaacagactatgttcgtgtgcaacaccttgcagcctgggtgtaatcaggcatgctacgataaagcattccctatatcacatatccgatattgggtatttcagattattatggtctgtaccccgtccctttgttttataacttacagcgtccaccagtctgccaaacagaaggagagacggtatagcaccgtgtacctcactttggataaggaccaggatagcctgaaacgcgatgaaagcaagaagataaagaacacaattgttaacggggtgcttcagaacactgaaaattcaacaaaggaggcggagccggactgcctcgaggtgaaggagattccaaactctgccatgcgaacgacgaaaagcaagatgagaagacaagagggaatttcaagattctacataatccaggtggtattccgaaacgctctcgagattggattcttggttgggcagtatttcttgtacgggtttaatgtgcctagtgtatatgagtgcgacagatacccttgtatcgaagatgtagaatgttacgtgtctcgcccaacggaaaaaactgtgtttcttgtttttatgttcgcggttagtggcttttgtgtagtcctcaacctcgcagagctgaaccatcttgggtggcgaaagattaaaaccgccgtgcgcggcgtccaggcacgacggaaaagcatctatgaaatcagaaacaaggacctcccaagaatgtccgtgcccaacttcggcaggacgcagtcaagtgattctgcctatgta

SEQ ID NO: 26 - DNA, 912 bp, synthetic construct
  source 1..912: mol_type = other DNA
atgggggagtggactatcttggagaggctgcttgaagctgctgtgcaacagcactcaaccatgattgggcggattcttttgactgttgttgtgatctttcggattctcatcgttgctatcgtcggagagaccgtttacgatgacgaacaaacaatgtttgtgtgtaacacccttcaaccagggtgtaaccaagcatgttatgacaaggcatttccaatttcacatatccgatactgggtcttccaaatcataatggtgtgcacaccatccctctgctttattacatatagcgtacaccagagcgcaaaacaaaaggagcggagatacagtaccgtttacttgacactggacaaagaccaagactccctgaaacgggacgagtcaaagaagataaagaacactatcgttaatggagtacttcaaaacacagagaacagtaccaaagaagccgagcccgattgccttgaagtcaaagagattcctaatagtgcaatgagaaccacaaaaagcaaaatgcgcaggcaggaaggtatctcacgcttctacatcatccaagtcgtcttccgcaacgccttggaaatcgggttcttggtcggacaatactttctctatgggtttaatgtcccaagcgtgtacgagtgtgaccgatacccctgcatcgaagacgttgagtgctatgtgtctcgccccaccgagaaaaccgtcttcctggtgttcatgtttgccgtcagtggcttttgtgtggttcttaatctcgctgagcttaaccatctggggtggcggaagattaagactgcagttagaggagtacaagcccgacgaaagtcaatatacgagattaggaataaagatcttcctagaatgagcgtgcccaactttgggcgcacacagagctcagacagcgcttatgta

SEQ ID NO: 27 - DNA, 1656 bp, synthetic construct
  source 1..1656: mol_type = other DNA
atgggagaatggactatcctggaacgactcctggaagcggctgtgcagcagcactcaacgatgataggtcggattcttctgacagtggtcgtaatatttagaattctcattgtagccattgtgggagaaacagtatatgaagatgaacaagcaatgttcatctgtaacactctgcaacctggttgtaatcaagcttgctacgataaagcatttcccataagccatattagatattgggtttttcagataatccttgtctgtactccatctctttgcttcattacttactcagtgcatcaatctgccaagcagaaggaccgcagatacagcttcctttatcccataatggagcgcgattatggggggagagacggtgctcgaaaactccggaatataaatgggatcttggtgcaacatgggggggacggagggggaggtaaagaggagcccgactgtctcgaagtcaaagagatacctaatgctccccgcgggctcacccacggtaaatcctctaaggtaagaaggcaggagggaataagtcggttttacattatccaagtcgttttcaggaacgctctcgaaatcggatttcttgctggacagtatttcctgtacggtttttccgttcctggaatattcaagtgcgatagatacccttgcttgaaaaaagttgaatgctacgtgagccgacccacagagaagacagttttccttgtattcatgttcgccgtgtcagggatctgtgtcgtcctcaatttggccgaattgaaccatttgggctggcgcaagataaaggcggcaataagaggtgtccaggctcgaagaaagtcaatttgtgaaatcagaaaaaaagacatggcgcacctctcacagccgcctaatttgggacgaacacagtcaagcgagagtgcttatgtcgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 28 - DNA, 1659 bp, synthetic construct
  source 1..1659: mol_type = other DNA
atgggtgagtggactatacttgaacgcctgcttgaggctgctgtgcaacagcactcaacaatgattggacgcatcttgttgacagttgttgttatattccgcatcctgatcgtcgccattgtgggggaaactgtctacgaggatgagcaggctatgttcatttgcaatacattgcagcccggctgcaaccaggcttgttacgataaggcctttccaatctctcatatacggtactgggtattccaaattattcttgtttgtacaccctccctctgtttcataacctattctgtacaccagtccgctaaacagaaagatcgccgatacagcttcctgtaccccataatggaacgcgactatggaggtagagacggggcacgaaaactgcgcaacataaatggtattctggtgcagcatggcggtgatggagggggtggaaaggaagaaccagactgtttggaagtgaaggagattcccaatgcacctcgcggacttactcacggaaaatcaagcaaggtacgacgacaagaggggatcagcaggttctatatcattcaggtggtctttagaaatgctttggaaattggttttctggcaggccagtattttctttatggtttcagtgtacctggcatattcaagtgcgataggtatccatgcttgaaaaaagtagagtgttatgtgtctagacctaccgaaaagactgtcttccttgtctttatgttcgctgtttcaggtatctgcgtcgttctcaacctggctgagctcaaccatttgggctggagaaagataaaggcagcaatccgcggcgtgcaggcccggagaaagtctatctgtgagattaggaaaaaagatatggcccacctttcccagccccctaacctgggaaggacacaaagctcagaaagtgcttacgtcgagctcgatccaccggtcgttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 29 - DNA, 1641 bp, synthetic construct
  source 1..1641: mol_type = other DNA
atgggtgagtggacaatccttgagaggctgctcgaagccgccgtccagcagcacagcaccatgataggtcgcatactgttgacagtagtagttatcttcagaatccttatcgtagctatcgttggggaaaccgtctatgacgatgaacagactatgttcgtgtgcaacaccttgcagcctgggtgtaatcaggcatgctacgataaagcattccctatatcacatatccgatattgggtatttcagattattatggtctgtaccccgtccctttgttttataacttacagcgtccaccagtctgccaaacagaaggagagacggtatagcaccgtgtacctcactttggataaggaccaggatagcctgaaacgcgatgaaagcaagaagataaagaacacaattgttaacggggtgcttcagaacactgaaaattcaacaaaggaggcggagccggactgcctcgaggtgaaggagattccaaactctgccatgcgaacgacgaaaagcaagatgagaagacaagagggaatttcaagattctacataatccaggtggtattccgaaacgctctcgagattggattcttggttgggcagtatttcttgtacgggtttaatgtgcctagtgtatatgagtgcgacagatacccttgtatcgaagatgtagaatgttacgtgtctcgcccaacggaaaaaactgtgtttcttgtttttatgttcgcggttagtggcttttgtgtagtcctcaacctcgcagagctgaaccatcttgggtggcgaaagattaaaaccgccgtgcgcggcgtccaggcacgacggaaaagcatctatgaaatcagaaacaaggacctcccaagaatgtccgtgcccaacttcggcaggacgcagtcaagtgattctgcctatgtagatccaccggtcgccaccatggtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaa

SEQ ID NO: 30 - DNA, 1644 bp, synthetic construct
  source 1..1644: mol_type = other DNA
atgggggagtggactatcttggagaggctgcttgaagctgctgtgcaacagcactcaaccatgattgggcggattcttttgactgttgttgtgatctttcggattctcatcgttgctatcgtcggagagaccgtttacgatgacgaacaaacaatgtttgtgtgtaacacccttcaaccagggtgtaaccaagcatgttatgacaaggcatttccaatttcacatatccgatactgggtcttccaaatcataatggtgtgcacaccatccctctgctttattacatatagcgtacaccagagcgcaaaacaaaaggagcggagatacagtaccgtttacttgacactggacaaagaccaagactccctgaaacgggacgagtcaaagaagataaagaacactatcgttaatggagtacttcaaaacacagagaacagtaccaaagaagccgagcccgattgccttgaagtcaaagagattcctaatagtgcaatgagaaccacaaaaagcaaaatgcgcaggcaggaaggtatctcacgcttctacatcatccaagtcgtcttccgcaacgccttggaaatcgggttcttggtcggacaatactttctctatgggtttaatgtcccaagcgtgtacgagtgtgaccgatacccctgcatcgaagacgttgagtgctatgtgtctcgccccaccgagaaaaccgtcttcctggtgttcatgtttgccgtcagtggcttttgtgtggttcttaatctcgctgagcttaaccatctggggtggcggaagattaagactgcagttagaggagtacaagcccgacgaaagtcaatatacgagattaggaataaagatcttcctagaatgagcgtgcccaactttgggcgcacacagagctcagacagcgcttatgtagagctcgatccaccggtcgttaacgtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaa

SEQ ID NO: 31 - DNA, 5611 bp, synthetic construct
  source 1..5611: mol_type = other DNA
atatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcatgggagaatggactatcctggaacgactcctggaagcggctgtgcagcagcactcaacgatgataggtcggattcttctgacagtggtcgtaatatttagaattctcattgtagccattgtgggagaaacagtatatgaagatgaacaagcaatgttcatctgtaacactctgcaaccgggttgtaatcaagcttgctacgataaagcatttcccataagccatattagatattgggtttttcagataatccttgtctgtactccatctctttgcttcattacttactcagtgcatcaatctgccaagcagaaggaccgcagatacagcttcctttatcccataatggagcgcgattatggggggagagacggtgctcgaaaactccggaatataaatgggatcttggtgcaacatgggggggacggagggggaggtaaagaggagcccgactgtctcgaagtcaaagagatacctaatgctccccgcgggctcacccacggtaaatcctctaaggtaagaaggcaggagggaataagtcggttttacattatccaagtcgttttcaggaacgctctcgaaatcggatttcttgctggacagtatttcctgtacggtttttccgttcctggaatattcgagtgcgatagatacccttgcttgaaagaagttgaatgctacgtgagccgacccacagagaagacagttttccttgtattcatgttcgccgtgtcagggatctgtgtcgtcctcaatttggccgaattgaaccatttgggctggcgcaagataaaggcggcaataagaggtgtccaggctcgaagaaagtcaatttgtgaaatcagaaaaaaagacatggcgcacctctcacagccgcctaatttgggacgaacacagtcaagcgagagtgcttatgtcgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagccc

SEQ ID NO: 32 - DNA, 5588 bp, synthetic construct
  source 1..5588: mol_type = other DNA
catgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatcatgggtgagtggacaatccttgagaggctgctcgaagccgccgtccagcagcacagcaccatgataggtcgcatactgttgacagtagtagttatcttcagaatccttatcgtagctatcgttggggaaaccgtctatgacgatgaacagactatgttcgtgtgcaacaccttgcagcctgggtgtaatcaggcatgctacgataaagcattccctatatcacatatccgatattgggtatttcagattattatggtctgtaccccgtccctttgttttataacttacagcgtccaccagtctgccaaacagaaggagagacggtatagcaccgtgtacctcactttggataaggaccaggatagcctgaaacgcgatgaaagcaagaagataaagaacacaattgttaacggggtgcttcagaacactgaaaattcaacaaaggaggcggagccggactgcctcgaggtgaaggagattccaaactctgccatgcgaacgacgaaaagcaagatgagaagacaagagggaatttcaagattctacataatccaggtggtattccgaaacgctctcgagattggattcttggttgggcagtatttcttgtacgggtttaatgtgcctagtgtatatgagtgcgacagatacccttgtatcaaagatgtagaatgttacgtgtctcgcccaacggaaaaaactgtgtttcttgtttttatgttcgcggttagtggcttttgtgtagtcctcaacctcgcagagctgaaccatcttgggtggcgaaagattaaaaccgccgtgcgcggcgtccaggcacgacggaaaagcatctatgaaatcagaaacaaggacctcccaagaatgtccgtgcccaacttcggcaggacgcagtcaagtgattctgcctatgtagatccaccggtcgccaccatggtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgc

SEQ ID NO: 33 - DNA, 5603 bp, synthetic construct
  source 1..5603: mol_type = other DNA
atatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatcatgggagaatggactatcctggaacgactcctggaagcggctgtgcagcagcactcaacgatgataggtcggattcttctgacagtggtcgtaatatttagaattctcattgtagccattgtgggagaaacagtatatgaagatgaacaagcaatgttcatctgtaacactctgcaacctggttgtaatcaagcttgctacgataaagcatttcccataagccatattagatattgggtttttcagataatccttgtctgtactccatctctttgcttcattacttactcagtgcatcaatctgccaagcagaaggaccgcagatacagcttcctttatcccataatggagcgcgattatggggggagagacggtgctcgaaaactccggaatataaatgggatcttggtgcaacatgggggggacggagggggaggtaaagaggagcccgactgtctcgaagtcaaagagatacctaatgctccccgcgggctcacccacggtaaatcctctaaggtaagaaggcaggagggaataagtcggttttacattatccaagtcgttttcaggaacgctctcgaaatcggatttcttgctggacagtatttcctgtacggtttttccgttcctggaatattcaagtgcgatagatacccttgcttgaaaaaagttgaatgctacgtgagccgacccacagagaagacagttttccttgtattcatgttcgccgtgtcagggatctgtgtcgtcctcaatttggccgaattgaaccatttgggctggcgcaagataaaggcggcaataagaggtgtccaggctcgaagaaagtcaatttgtgaaatcagaaaaaaagacatggcgcacctctcacagccgcctaatttgggacgaacacagtcaagcgagagtgcttatgtcgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagccc

SEQ ID NO: 34 - DNA, 5596 bp, synthetic construct
  source 1..5596: mol_type = other DNA
catgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcatgggtgagtggacaatccttgagaggctgctcgaagccgccgtccagcagcacagcaccatgataggtcgcatactgttgacagtagtagttatcttcagaatccttatcgtagctatcgttggggaaaccgtctatgacgatgaacagactatgttcgtgtgcaacaccttgcagcctgggtgtaatcaggcatgctacgataaagcattccctatatcacatatccgatattgggtatttcagattattatggtctgtaccccgtccctttgttttataacttacagcgtccaccagtctgccaaacagaaggagagacggtatagcaccgtgtacctcactttggataaggaccaggatagcctgaaacgcgatgaaagcaagaagataaagaacacaattgttaacggggtgcttcagaacactgaaaattcaacaaaggaggcggagccggactgcctcgaggtgaaggagattccaaactctgccatgcgaacgacgaaaagcaagatgagaagacaagagggaatttcaagattctacataatccaggtggtattccgaaacgctctcgagattggattcttggttgggcagtatttcttgtacgggtttaatgtgcctagtgtatatgagtgcgacagatacccttgtatcgaagatgtagaatgttacgtgtctcgcccaacggaaaaaactgtgtttcttgtttttatgttcgcggttagtggcttttgtgtagtcctcaacctcgcagagctgaaccatcttgggtggcgaaagattaaaaccgccgtgcgcggcgtccaggcacgacggaaaagcatctatgaaatcagaaacaaggacctcccaagaatgtccgtgcccaacttcggcaggacgcagtcaagtgattctgcctatgtagatccaccggtcgccaccatggtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgc

SEQ ID NO: 35 - DNA, 7020 bp, synthetic construct
  source 1..7020: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccgccaccatgggtgagtggactatacttgaacgcctgcttgaggctgctgtgcaacagcactcaacaatgattggacgcatcttgttgacagttgttgttatattccgcatcctgatcgtcgccattgtgggggaaactgtctacgaggatgagcaggctatgttcatttgcaatacattgcagcccggctgcaaccaggcttgttacgataaggcctttccaatctctcatatacggtactgggtattccaaattattcttgtttgtacaccctccctctgtttcataacctattctgtacaccagtccgctaaacagaaagatcgccgatacagcttcctgtaccccataatggaacgcgactatggaggtagagacggggcacgaaaactgcgcaacataaatggtattctggtgcagcatggcggtgatggagggggtggaaaggaagaaccagactgtttggaagtgaaggagattcccaatgcacctcgcggacttactcacggaaaatcaagcaaggtacgacgacaagaggggatcagcaggttctatatcattcaggtggtctttagaaatgctttggaaattggttttctggcaggccagtattttctttatggtttcagtgtacctggcatattcgagtgcgataggtatccatgcttgaaagaagtagagtgttatgtgtctagacctaccgaaaagactgtcttccttgtctttatgttcgctgtttcaggtatctgcgtcgttctcaacctggctgagctcaaccatttgggctggagaaagataaaggcagcaatccgcggcgtgcaggcccggagaaagtctatctgtgagattaggaaaaaagatatggcccacctttcccagccccctaacctgggaaggacacaaagctcagaaagtgcttacgtcgagctcgatccaccggtcgttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 36 - DNA, 7005 bp, synthetic construct
  source 1..7005: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccgccaccatgggggagtggactatcttggagaggctgcttgaagctgctgtgcaacagcactcaaccatgattgggcggattcttttgactgttgttgtgatctttcggattctcatcgttgctatcgtcggagagaccgtttacgatgacgaacaaacaatgtttgtgtgtaacacccttcaaccagggtgtaaccaagcatgttatgacaaggcatttccaatttcacatatccgatactgggtcttccaaatcataatggtgtgcacaccatccctctgctttattacatatagcgtacaccagagcgcaaaacaaaaggagcggagatacagtaccgtttacttgacactggacaaagaccaagactccctgaaacgggacgagtcaaagaagataaagaacactatcgttaatggagtacttcaaaacacagagaacagtaccaaagaagccgagcccgattgccttgaagtcaaagagattcctaatagtgcaatgagaaccacaaaaagcaaaatgcgcaggcaggaaggtatctcacgcttctacatcatccaagtcgtcttccgcaacgccttggaaatcgggttcttggtcggacaatactttctctatgggtttaatgtcccaagcgtgtacgagtgtgaccgatacccctgcatcaaagacgttgagtgctatgtgtctcgccccaccgagaaaaccgtcttcctggtgttcatgtttgccgtcagtggcttttgtgtggttcttaatctcgctgagcttaaccatctggggtggcggaagattaagactgcagttagaggagtacaagcccgacgaaagtcaatatacgagattaggaataaagatcttcctagaatgagcgtgcccaactttgggcgcacacagagctcagacagcgcttatgtagagctcgatccaccggtcgttaacgtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 37 - DNA, 7020 bp, synthetic construct
  source 1..7020: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccgccaccatgggtgagtggactatacttgaacgcctgcttgaggctgctgtgcaacagcactcaacaatgattggacgcatcttgttgacagttgttgttatattccgcatcctgatcgtcgccattgtgggggaaactgtctacgaggatgagcaggctatgttcatttgcaatacattgcagcccggctgcaaccaggcttgttacgataaggcctttccaatctctcatatacggtactgggtattccaaattattcttgtttgtacaccctccctctgtttcataacctattctgtacaccagtccgctaaacagaaagatcgccgatacagcttcctgtaccccataatggaacgcgactatggaggtagagacggggcacgaaaactgcgcaacataaatggtattctggtgcagcatggcggtgatggagggggtggaaaggaagaaccagactgtttggaagtgaaggagattcccaatgcacctcgcggacttactcacggaaaatcaagcaaggtacgacgacaagaggggatcagcaggttctatatcattcaggtggtctttagaaatgctttggaaattggttttctggcaggccagtattttctttatggtttcagtgtacctggcatattcaagtgcgataggtatccatgcttgaaaaaagtagagtgttatgtgtctagacctaccgaaaagactgtcttccttgtctttatgttcgctgtttcaggtatctgcgtcgttctcaacctggctgagctcaaccatttgggctggagaaagataaaggcagcaatccgcggcgtgcaggcccggagaaagtctatctgtgagattaggaaaaaagatatggcccacctttcccagccccctaacctgggaaggacacaaagctcagaaagtgcttacgtcgagctcgatccaccggtcgttaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 7005 bp, synthetic construct
  source 1..7005: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccgccaccatgggggagtggactatcttggagaggctgcttgaagctgctgtgcaacagcactcaaccatgattgggcggattcttttgactgttgttgtgatctttcggattctcatcgttgctatcgtcggagagaccgtttacgatgacgaacaaacaatgtttgtgtgtaacacccttcaaccagggtgtaaccaagcatgttatgacaaggcatttccaatttcacatatccgatactgggtcttccaaatcataatggtgtgcacaccatccctctgctttattacatatagcgtacaccagagcgcaaaacaaaaggagcggagatacagtaccgtttacttgacactggacaaagaccaagactccctgaaacgggacgagtcaaagaagataaagaacactatcgttaatggagtacttcaaaacacagagaacagtaccaaagaagccgagcccgattgccttgaagtcaaagagattcctaatagtgcaatgagaaccacaaaaagcaaaatgcgcaggcaggaaggtatctcacgcttctacatcatccaagtcgtcttccgcaacgccttggaaatcgggttcttggtcggacaatactttctctatgggtttaatgtcccaagcgtgtacgagtgtgaccgatacccctgcatcgaagacgttgagtgctatgtgtctcgccccaccgagaaaaccgtcttcctggtgttcatgtttgccgtcagtggcttttgtgtggttcttaatctcgctgagcttaaccatctggggtggcggaagattaagactgcagttagaggagtacaagcccgacgaaagtcaatatacgagattaggaataaagatcttcctagaatgagcgtgcccaactttgggcgcacacagagctcagacagcgcttatgtagagctcgatccaccggtcgttaacgtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggcctccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - DNA, 7252 bp, synthetic construct
  source 1..7252: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatcctctagagtcgactccggaataacttcgtataggatactttatacgaagttatgcagaatggtagctggattgtagctgctattagcaatatgaaacctcttaataacttcgtatagcatacattatacgaagttatggcgcgccttacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgctgccgtcctcgatgttgtggcgggtcttgaagttcaccttgatgccgttcttctgcttgtcggcggtgatatagaccttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtaggtcaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcacgttaacgaccggtggatcgagctcgacgtaagcactttctgagctttgtgtccttcccaggttagggggctgggaaaggtgggccatatcttttttcctaatctcacagatagactttctccgggcctgcacgccgcggattgctgcctttatctttctccagcccaaatggttgagctcagccaggttgagaacgacgcagatacctgaaacagcgaacataaagacaaggaagacagtcttttcggtaggtctagacacataacactctacttttttcaagcatggatacctatcgcacttgaatatgccaggtacactgaaaccataaagaaaatactggcctgccagaaaaccaatttccaaagcatttctaaagaccacctgaatgatatagaacctgctgatcccctcttgtcgtcgtaccttgcttgattttccgtgagtaagtccgcgaggtgcattgggaatctccttcacttccaaacagtctggttcttcctttccaccccctccatcaccgccatgctgcaccagaataccatttatgttgcgcagttttcgtgccccgtctctacctccatagtcgcgttccattatggggtacaggaagctgtatcggcgatctttctgtttagcggactggtgtacagaataggttatgaaacagagggagggtgtacaaacaagaataatttggaatacccagtaccgtatatgagagattggaaaggccttatcgtaacaagcctggttgcagccgggctgcaatgtattgcaaatgaacatagcctgctcatcctcgtagacagtttcccccacaatggcgacgatcaggatgcggaatataacaacaactgtcaacaagatgcgtccaatcattgttgagtgctgttgcacagcagcctcaagcaggcgttcaagtatagtccactcacccatggtggcgctagcataacttcgtataaagtatcctatacgaagttatttgccttaacccagaaattatcactgttattctttagaatggtgcaaagaataacttcgtataatgtatgctatacgaagttatgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - DNA, 7237 bp, synthetic construct
  source 1..7237: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatcctctagagtcgactccggaataacttcgtataggatactttatacgaagttatgcagaatggtagctggattgtagctgctattagcaatatgaaacctcttaataacttcgtatagcatacattatacgaagttatggcgcgccttacttgtacagctcgtccatgccgccggtggagtggcggccctcggcgcgttcgtactgttccacgatggtgtagtcctcgttgtgggacacgatgtccaacttgatgtcgacgatgtaggcgccgggcagctgcacgggcttcttggccttgtaggtggtcttgacctcggcggcgtagtggccgccgtccttcagcttcagcctcttcttgatctcgctcttcagggcgccgtcctcggggtacatccgctcctcggaggcctcccagcccatggtcttcttctgcattacggggccgtcggaggggaagttggtgccgcgcagcttcaccttgtagatgaacacgccgtcctgcagggaggagtcctggttaacgtgaataatgccgccgtcctcgaagttcatcacgcgctcccacctgaagccctcggggaaggacagcttgaagtagtcggggatgtcggctgggtgcttaatgtagaccttggagccgtacatgaactgaggggacaggatgtcccaggcgaagggcagggggccacccttggtcaccttcagcttagcggtctgaaaggcctcgtaggggcggccctcgccctcgccctcgatctcgaactcgtggccgttcacggagccctccatgtgcaccttgaagcgcatgaactccttgatgatggccatgttattctcctcgcccttgctcacgttaacgaccggtggatcgagctctacataagcgctgtctgagctctgtgtgcgcccaaagttgggcacgctcattctaggaagatctttattcctaatctcgtatattgactttcgtcgggcttgtactcctctaactgcagtcttaatcttccgccaccccagatggttaagctcagcgagattaagaaccacacaaaagccactgacggcaaacatgaacaccaggaagacggttttctcggtggggcgagacacatagcactcaacgtcttcgatgcaggggtatcggtcacactcgtacacgcttgggacattaaacccatagagaaagtattgtccgaccaagaacccgatttccaaggcgttgcggaagacgacttggatgatgtagaagcgtgagataccttcctgcctgcgcattttgctttttgtggttctcattgcactattaggaatctctttgacttcaaggcaatcgggctcggcttctttggtactgttctctgtgttttgaagtactccattaacgatagtgttctttatcttctttgactcgtcccgtttcagggagtcttggtctttgtccagtgtcaagtaaacggtactgtatctccgctccttttgttttgcgctctggtgtacgctatatgtaataaagcagagggatggtgtgcacaccattatgatttggaagacccagtatcggatatgtgaaattggaaatgccttgtcataacatgcttggttacaccctggttgaagggtgttacacacaaacattgtttgttcgtcatcgtaaacggtctctccgacgatagcaacgatgagaatccgaaagatcacaacaacagtcaaaagaatccgcccaatcatggttgagtgctgttgcacagcagcttcaagcagcctctccaagatagtccactcccccatggtggcgctagcataacttcgtataaagtatcctatacgaagttatttgccttaacccagaaattatcactgttattctttagaatggtgcaaagaataacttcgtataatgtatgctatacgaagttatgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2025/0186620 A1
(43) Pub. Date: Jun. 12, 2025

(54) COMPOSITIONS FOR AND METHODS OF LONG-TERM INTEGRATION OF CIRCUITS USING CONNEXINS

(71) Applicant: Duke University, Durham, NC (US)

(72) Inventors: Kafui Dzirasa, Durham, NC (US); Elizabeth Ransey, Durham, NC (US); Kirill Chesnov, Durham, NC (US); Rainbo Hultman, Durham, NC (US)

(21) Appl. No.: 18/686,116

(22) PCT Filed: Aug. 22, 2022
(86) PCT No.: PCT/US2022/075288

§ 371 Date: Feb. 23, 2024

Related U.S. Application Data
(60) Provisional application No. 63/278,289, filed on Nov. 11, 2021.
(60) Provisional application No. 63/235,940, filed on Aug. 23, 2021.

(51) Int. Cl. A61K 48/00; A61P 25/14; C07K 14/46; C12N 15/86

(52) U.S. Cl. CPC A61K 48/0058; A61P 25/14; C07K 14/461; C12N 15/86; C12N 2750/14143

(57) ABSTRACT

Disclosed herein are compositions for use in methods of precision editing circuits using designer connexins.

### I. CROSS-REFERENCE TO RELATED APPLICATIONS

This application claims priority to U.S. Provisional Application No. 63/235,940 filed 23 Aug. 2021 and U.S. Provisional Application No. 63/278,298 filed 11 Nov. 2021, each of which is incorporated herein in its entirety.

### STATEMENT REGARDING FEDERALLY SPONSORED RESEARCH OR DEVELOPMENT

This invention was made with government support under U01HL134764, R01HL132389, R01HL126524, R21EY029451, R01MH120158, and R01MH125430 awarded by the National Institutes of Health. The government has certain rights in the invention.

### II. REFERENCE TO THE SEQUENCE LISTING

The Sequence Listing submitted 22 Aug. 2022 as a .xml file named “22_2047_WO_Sequence_Listing”, created on 22 Aug. 2022 and having a size of 116 kilobytes is hereby incorporated by reference pursuant to 37 C.F.R. § 1.52(e)(5).

### III. BACKGROUND

Gap junctions (electrical synapses) enable direct flow of ions and small molecules between two cells and play a prominent role in broadly synchronizing electrical activity in many organs such as the heart and the brain. To achieve such synchrony, gap junction consists of two docked segments called hemichannels, embedded in the membranes of two adjoining cells. Each hemichannel, in turn, is an oligomer consisting of six monomeric proteins called connexins (Cx), of which there are 21 isoforms in humans. Many Cxs can form single-isoform hemichannels. Gap junctions are membrane spanning channels that connect the cytoplasm of apposed cells, allowing for the passage of small molecules and ions. Gap junctions (GJs) play critical roles in tissue homeostasis, cellular signaling, and intercellular communication (i.e., ionic and small molecule exchange). Mutations in any of the Cx genes can lead to pathological changes—including congenital sensorineural deafness, Charcot-Marie-Tooth disorder, and Oculodentodigital dysplasia (ODDD). Additionally, expression level changes have been associated with epileptic conditions. The coordination of activity between brain cells is a key determinant of neural circuit function; nevertheless, approaches that selectively regulate communication between two distinct cellular components of a circuit, while leaving the activity of the presynaptic brain cell undisturbed remain sparse.

Consequently, the present disclosure provides compositions for and methods of precision editing circuits using engineered connexins.

### IV. BRIEF DESCRIPTION OF THE FIGURES

FIG. 1A-FIG. 1F detail the screening used to identify mutant connexin hemichannel pair that exhibited exclusively heterotypic docking. FIG. 1A is a schematic outlining the limitation of introducing ectopic wild type connexin hemichannels (pink rectangles) as a method for selectively modulating precise neural circuits composed by brown and light green neurons (left). Note that connexin hemichannels yielded off-target electrical synapses between pre-synaptic neurons, and thus off-target modulation of other circuits (see, e.g., FIG. 11A and FIG. 11B as the wild-type Cx protein killed mice, likely due to hyperconnectivity between the wrong neurons, which is important as the current state of the art uses wild-type proteins and works in animals with a single cell per cell type, but clearly not with mammals). Putative strategy for deploying exclusively heterotypic docking hemichannels (green and red rectangles) to selectively modulate precise neural circuits (right). Note the rectification of this putative gap junction. FIG. 1B shows counterpart fluorescently labeled connexin proteins (i.e., C-terminally fused mEmerald or RFP670) as expressed in different cell populations. Cells were then combined and accessed for opposing hemichannel internalization using flow cytometry (left). Representative flow cytometry plot for wild type Cx34.7/Cx35 pair is shown on top, and representative flow cytometry plot for Cx36/Cx45 pair is shown on bottom (right). Pink square highlights portion of individual cells labeled by two distinct fluorescent proteins.

FIG. 1C shows a schematic of Morone Americana Cx34.7 and Cx35 extracellular loop mutations used to screen for novel, heterotypic exclusive hemichannels. Positions and mutations unique to Cx34.7 and Cx35 are shown in green and red, respectively. Positions and mutations common to both proteins are shown in black. FIG. 1D-FIG. 1E show circular plot showing homotypic FETCH results for Cx34.7 mutations (FIG. 1D) and Cx35 mutations (FIG. 1E). Bar graphs show the effect size (portion of dual labeled cells) of homotypic mutant combinations relative to the heterotypic pairing of human Cx36 and Cx45 which failed to dock. Mutations that disrupted docking are highlighted by black arrows. Purple circle corresponds to zero effect size. Black line corresponds to scale bar for effect size. FIG. 1F shows heterotypic FETCH results for Cx34.7 and Cx35 mutant protein combinations. Bar graphs show the effect size of homotypic mutant combinations relative to the wild type Cx34.7 and Cx35 pair.

FIG. 2 shows an integrated approach used to model connexin hemichannel docking. This approach consists of seven integrated components: (i) homology model generation, (ii) in silico protein mutagenesis, (iii) embedding of proteins in a lipid bilayer and aqueous solution, (iv) system minimization, equilibration, and molecular dynamics simulation, (v) residue-wise energy calculation, (vi) in vitro protein mutagenesis, and (vii) FETCH validation.

FIG. 3A-FIG. 3C show the model of extracellular loop-2 (EL2) residues mediating homotypic and heterotypic hemichannel connexin docking. EL2-to-EL2 interactions predicted between wild type Cx34.7 and Cx35 using homology modeling. Residues predicted to form strong attractive/repulsive interactions are highlighted in blue/red respectively (top). Contact plots for EL2-to-EL2 interactions produced by molecular dynamics simulation (middle), and summary of interactions predicted to stabilize hemichannels pairs (bottom). Plots are shown for homotypic Cx34.7 interactions (FIG. 3A), homotypic Cx35 interactions (FIG. 3B), and heterotypic Cx34.7 and Cx35 interactions (FIG. 3C).

FIG. 4A-FIG. 4F shows that engineered Cx34.7 and Cx35 mutants to show heterotypic, but not homotypic, hemichannel docking. FIG. 4A-FIG. 4C shows homology models predicting EL2-to-EL2 residue interactions for Cx34.7 and Cx35 mutant hemichannels. Residues predicted to form strong attractive/repulsive interactions are highlighted in blue/red respectively (top). Contact plots for EL2-to-EL2 interactions produced by molecular dynamics simulation (middle), and summary of interactions predicted to stabilize/destabilize hemichannels pairs (bottom). Plots are shown for homotypic Cx34.7_{E214K/E223K} (FIG. 4A), homotypic Cx35_{K221E} (FIG. 4B), and heterotypic Cx34.7_{E214K/E223K} (FIG. 4C) and Cx35_{K221E} interactions (Cx34.7 residues are shown along the y-axis and Cx35 residues are shown along the x axis). FIG. 4D-FIG. 4F shows confocal images of heterotypic connexin pairs—Cx34.7_{WT}/Cx35_{WT} (FIG. 4D), Cx34.7_{E214K/E223K/E225K}/Cx35_{K221E} (FIG. 4E), and Cx34.7_{E214K/E223K}/Cx35_{K221E} (FIG. 4F) expressed in HEK 293FT cells. All Cx34.7 and Cx35 proteins are expressed as mEmerald and RFP670 fusion proteins, respectively. White arrows highlight dual fluorescent labeled vesicles. Note the cytoplasmic localization of Cx34.7_{E214K/E223K/E225K} in panel (FIG. 4E).

FIG. 5A-FIG. 5D shows ectopic connexin hemichannels couple C. elegans neurons and recode thermal preference. FIG. 5A shows a schematic of the AFD→AIY synaptic communication and expressed temperature preference. The AFD thermosensory neuron has a robust calcium response to warming stimuli. C. elegans raised in the presence of food at 15° C., or animals with a Protein Kinase C (PKC)-1 gain-of-function mutation, move towards cooler temperatures when placed on a thermal gradient (top). Ectopic expression of connexin hemichannels between AFD and AIY results in synchronization of the signal to AIY and promotes warm-seeking behavior (bottom). FIG. 5B shows calcium traces of neurons expressing ectopic connexin hemichannel pairs (left). Baseline AFD and AIY responses are also shown. Each panel depicts the average trace a group (top, data shown as mean±SEM), heatmaps of individual animals (middle), and the temperature stimulus (bottom). Behavioral traces for each group are shown on the right. Traces are shown for C. elegans homotypically expressing wild type connexin hemichannels (pink highlight), heterotypically expressing the mutant pair (tan highlight), and homotypically expressing mutant connexin hemichannels (cyan highlight). FIG. 5C shows that a portion of animals showing neuronal calcium responses based on the traces shown in FIG. 5B;***p<0.0005 using Fisher's exact test for penetrance. Error bars denote 95% C.I. FIG. 5D shows thermotaxis indices corresponding to experimental groups. Each data point represents the thermotaxis preference index of a separate assay (12-15 animals/assay), with the median for each group plotted denoted by a black horizontal. **p<0.005; ***p<0.0005; Error bars=95% C.I.

FIG. 6A-FIG. 6F shows LinCx edits microcircuit relevant dynamics in mice. FIG. 6A shows prefrontal cortex microcircuit composed by an excitatory pyramidal neuron (PYR) and a parvalbumin expressing fast-spiking interneuron (PV+). The tan circle highlights the target for synaptic editing (top). PV-Cre mice were co-infected with AAV-CaMKII-Cx34.7_{M1} and AAV-DIO-Cx35_{M11} bilaterally (bottom). Control mice were infected with a Cx34.7_{M1} or Cx35_{M1} pair of viruses which expressed the same hemichannel in both cell types. Mice were subsequently implanted in microwires in prefrontal cortex. FIG. 6B shows representative local field potential recorded from prefrontal cortex (top). Power spectrograms shown theta (4 Hz-10 Hz) and high frequency activity (80 Hz-200 Hz), which corresponds to PYR and PV+ neuron firing, respectively (middle). Microcircuit function is represented by the coupling between the phase of theta oscillations (black) and the amplitude of high frequency oscillations (red, bottom). FIG. 6C shows the distribution of theta-high frequency coupling scores observed across uninfected C57BL/6j mice (n=29, black), PV-Cre mice co-infected with a Cx34.7_{M1} (n=8) or Cx35_{M1} (n=8) pair of viruses (blue), or mice co-infected with a docking Cx34.7_{M1}/Cx35_{M1} pair (n=12, tan). Mice co-infected with the Cx34.7_{M1}/Cx35_{M1} pair showed significantly higher theta-high frequency oscillation coupling than non-edited mice (U=1161; p=0.0025 using one tailed rank-sum test).

FIG. 6D shows schematic of Infralimbic cortex→Medial dorsal thalamus circuit targeted for editing, and viral infection strategy. FIG. 6E shows a schematic of behavioral testing to quantify stress induced behavioral adaptation. FIG. 6F shows the immobility time and distance travelled during repeat tail suspension (left) and open field testing (right). Mice co-infected with Cx34.7_{M1} or Cx35_{M1} in homotypic non-docking configurations showed stress induced behavioral adaptation during repeat TST testing (tan), while mice co-infected with the functional Cx34.7_{M1}/Cx35_{M1} pair did not (blue). No behavioral differences were observed in the open field; \hat{ } denotes Group effect; #denotes Group×Day interaction effect using mixed effects model ANOVA.

FIG. 7A-FIG. 7B shows the complementary utility of LinCx compared to widely adopted neural modulation approaches in selectively targeting a spatially and cell-type pair defined circuit. FIG. 7A shows electrical stimulation activates many cell types and pass-through fibers within a tissue volume. Stimulation also modulates cellular activity independent of the context of input fibers, and potentially drives retrograde activation of cellular inputs. Open-loop optogenetic stimulation of neuronal soma modulates cell-type specific activity independent of the context of input fibers. Optogenetic terminal stimulation enables modulation of connections between brain regions, but potentially activates multiple circuits defined by distinct post-synaptic cell types, and potentially drives retrograde activation of target axons. DREADDs modulate target cells, but potentially modulate the response of target cells to their input fibers. FIG. 7B shows LinCx enables selective targeting based on a cell-type defined pair and selective modulation of the post-synaptic cell-type based on the activity context of the pre-synaptic cell.

FIG. 8 shows confocal maximum intensity projections of C. elegans expressing the Cx34.7_{M1}::Cx35_{M1} pair, related to FIG. 5A-FIG. 5D. GFP-tagged Cx34.7M1 is expressed in the AFD neuron, with puncta along its axon (left). mCherry-tagged Cx35M1 is expressed in the AIY neuron, with puncta along its neurite (middle). Composite image showing the colocalizing GFP/mCherry puncta (right). Scale bar is 10 μm. White arrows highlight puncta.

FIG. 9 shows the time course of Cx34.7 expression and trafficking in prelimbic cortex soma and terminals, related for FIG. 6. Five C57BL/6J mice were infected with AAV9-CaMKII-Cx34.7_{M1}-mEmerald in Prelimbic cortex. Histology was performed 3 weeks (n=1 mouse), 5 weeks (n=2 mice), or 7 weeks (n=2 mice) after viral surgeries.

FIG. 10A-FIG. 10B shows representative histological images for mouse editing experiments, related to FIG. 6. FIG. 10A shows representative histological image of prelimbic cortex in A) Cx34.7_{M1}/Cx35_{M1} infected mouse (left) and a Cx34.7_{M1}/Cx34.7_{M1} control (middle). White arrows highlight electrode tracks. For mice included in the prelimbic cortex editing experiment, individual electrode wires were distributed within the area highlighted by the red rectangle. FIG. 10B shows histological image of mouse injected with AAV9-CaMKII-Cx34.7_{M1}-mEmerald in infralimbic cortex (left). White arrows highlight viral injection tracks. Image showing medial dorsal thalamus in mouse injected with AAV9-CaMKII-Cx34.7_{M1}-mEmerald in infralimbic cortex and AAV9-CaMKII-Cx35_{M1}-mApple in medial dorsal thalamus (middle). Composite confocal image of medial dorsal thalamus for mouse expressing Cx34.7_{M1} and Cx35_{M1} (right). Arrows highlight pucta with co-localization of mEmerald and mApple.

FIG. 11A shows the strategy for injection the ventral hippocampus of mice (n=30) with either a naturally occurring version of Cx35 (Cx35_{WT}) or designer Cx35 (Cx35_{M1}) as either AAV9-CaMKII-Cx35_{WT} construct or AAV9-CaMKII-Cx35_{M1} construct. FIG. 11B shows the survival % for mice injected with AAV9-CaMKII-Cx35_{M1} (n=16), AAV9-CaMKII-Cx35_{WT} at 300 μL (n=6), or AAV9-CaMKII-Cx35_{WT} at 500 μL (n=8). The WT protein killed mice, likely due to hyperconectivity between the wrong neurons. This is important because the current state of the art (i) uses WT proteins and (ii) works in animals with a single cell per cell type, but clearly not with mammals.

### V. BRIEF SUMMARY

Disclosed herein is an isolated nucleic acid molecule comprising a nucleic acid sequence encoding a designer connexin protein. Disclosed herein is an isolated nucleic acid molecule comprising a nucleic acid sequence encoding a designer Cx34.7 connexin protein. Disclosed herein is an isolated nucleic acid molecule, comprising: a nucleic acid sequence encoding a designer Cx35 connexin protein.

Disclosed herein is a designer connexin protein. Disclosed herein is a designer connexin protein comprising the Cx34.7 connexin protein of Morone americana having a substitution at amino acid 214 and at amino acid 223. Disclosed herein is a designer connexin protein comprising the Cx35 connexin protein of Morone americana having a substitution at amino acid 221.

Disclosed herein is a fluorescently labeled designer connexin protein. Disclosed herein is a fluorescently labeled designer Cx34.7 protein. Disclosed herein is a fluorescently labeled designer Cx34.7_{M1} protein. Disclosed herein is a fluorescently labeled designer Cx35 protein. Disclosed herein is a fluorescently labeled designer Cx35_{M1} protein. Disclosed herein is a pair of designer connexin proteins that can be used in a disclosed method.

Disclosed herein is a pair of designer non-mammalian connexin proteins that can be used in a disclosed method. Disclosed herein is a pair of designer connexin proteins that can be used in a disclosed method of long-term integration of circuits.

Disclosed herein are docked hemichannels comprising a first disclosed designer connexin and a second disclosed designer connexin. Disclosed herein are docked hemichannels comprising a first designer connexin protein and a second designer connexin protein. Disclosed herein are hemichannel combinations comprising a first labeled designer connexin and a second labeled designer connexin. Disclosed herein is a labeled connexosome comprising a first disclosed connexin and a second disclosed connexin. Disclosed herein are docked hemichannels comprising a Cx34.7 designer connexin protein and a Cx35 designer connexin. Disclosed herein are docked hemichannels comprising a Cx34.7_{M1} designer connexin and a Cx35_{M1} designer connexin protein.

Disclosed herein is a connexosome comprising a first disclosed designer connexin protein and a second disclosed designer connexin protein. Disclosed herein is a connexosome comprising a disclosed Cx34.7 designer connexin protein and a disclosed Cx35 designer connexin protein.

Disclosed herein is a connexosome comprising a disclosed Cx34.7_{M1} connexin protein and a disclosed Cx35_{M1} connexin protein. Disclosed herein is a labeled connexosome comprising a first disclosed designer connexin protein and a second disclosed designer connexin protein.

Disclosed herein is a synthetic gap junction comprising a first hemichannel heterotypically docked to a second hemichannel, wherein the first hemichannel comprises a first disclosed designer connexin protein, and wherein the second hemichannel comprises a second designer connexin protein. Disclosed herein is a synthetic gap junction comprising a first disclosed hemichannel and a second disclosed hemichannel. Disclosed herein is a synthetic gap junction comprising a hemichannel comprising a disclosed designer Cx34.7 connexin protein and a hemichannel comprising a disclosed designer Cx35 connexin protein. Disclosed herein is a synthetic gap junction comprising a hemichannel comprising a disclosed designer Cx34.7_{M1} connexin protein and a hemichannel comprising a disclosed designer Cx35_{M1} connexin protein.

Disclosed herein is a viral vector comprising a disclosed isolated nucleic acid molecule. Disclosed herein is a viral vector comprising an isolated nucleic acid molecule comprising a nucleic acid sequence encoding a designer connexin protein. Disclosed herein is a viral vector comprising an isolated nucleic acid molecule comprising a nucleic acid sequence encoding a designer Cx34.7 connexin protein. Disclosed herein is a viral vector comprising an isolated nucleic acid molecule comprising a nucleic acid sequence encoding a designer Cx35 connexin protein. Disclosed herein is a viral vector comprising an isolated nucleic acid molecule comprising a nucleic acid sequence encoding a designer Cx34.7_{M1} connexin protein. Disclosed herein is a viral vector comprising an isolated nucleic acid molecule comprising a nucleic acid sequence encoding a designer Cx35_{M1} connexin protein.

Disclosed herein is a method of precision editing a neural circuit the method comprising generating a synthetic gap junction between a targeted first type of cells and targeted second type of cells in the central nervous system in a subject in need thereof, wherein the synthetic gap junction facilitates electrical activity between the first type of cells and the second type of cells.

Disclosed herein is a method of precision editing a cellular circuit the method comprising generating a synthetic gap junction between a targeted first type of cells and targeted second type of cells in the cellular circuit in a subject in need thereof, wherein the synthetic gap junction facilitates electrical activity between the first type of cells and the second type of cells.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Connexins

Gap junctions are multimeric, transmembrane channels that play crucial roles in tissue homeostasis, cellular signaling, and the propagation of electrical current by enabling direct intercellular communication between adjacent, apposed cells via ionic and small molecule exchange (Alexander D B, et al. (2003) Curr Med Chem. 10:2045-2058)). Gap junctions are composed of connexin (Cx) proteins, which oligomerize into hexameric hemichannels (Falk M M, et al. (1997) EMBO J. 16:2703-2716; Ahmad S, et al. (1999) Biochem J. 339(Pt 2):247-253) and dock with compatible hemichannels of adjacent cells at the plasma membrane to form an intercellular pore connecting the two cells (Perkins G A, et al. (1998) J Mol Biol. 277:171-177). As shown in Table 1, connexins comprise a family of integral membrane proteins consisting of 21 unique isoforms in humans (Sohl G, et al. (2003) Cell Commun Adhes. 10:173-180; Sohl G, et al. (2004) Cardiovasc Res. 62:228-232), with at least one isoform expressed in virtually every tissue and major organ in the body (Goodenough D A, et al. (1996) Annu Rev Biochem. 65:475-502) resulting in distinct and overlapping isoform-specific expression patterns. Routinely, single cell-types simultaneously express two or more connexin isoforms, creating both opportunities for functional redundancy and complex, highly-regulated tissue communication (Laird D W. (2006) Biochem J. 394:527-543).

Connexins play significant roles in the normal physiology of the tissues in which they are expressed. Thus, mutations in any of the Cx genes can lead to pathological changes. For example, at least 100 mutations of the Connexin26 (Cx26) encoding gene (GJB2) accounts for half of all worldwide cases of congenital sensorineural deafness (Lee J R, et al. (2009) Expert Rev Mol Med. 11:e35; Scott C A, et al. (2011) Biochem J. 438:245-254). Charcot-Marie-Tooth, a motor and sensory neurodegenerative disorder, is associated with mutations of the Connexin32 (Cx32) gene (GJB1) (Nelis E, et al. (1999) Human Mutation. 13:11-28). At least 73 different mutations of the most ubiquitously expressed connexin, Connexin43 (Cx43) gene (GJA1), are associated with Oculodentodigital dysplasia (ODDD) (Laird D W. (2014) Febs Letters. 588:1339-1348), which is a rare, developmental disorder resulting in numerous morphological anomalies and neurological symptoms (De Bock M, et al. (2013) Front Pharmacol. 4:120). Additionally, aberrant expression levels or regulation of Cx43, the primary connexin expressed in the heart, are associated with cardiac arrythmias in the context of myocardial ischemia (Lerner D L, et al. (2000) Circulation. 101:547-552).

Connexin proteins share a conserved topology consisting of intracellular amino- and carboxy-termini, a cytoplasmic loop, 4 transmembrane (TM) helices, and 2 extracellular loops (ELs). Post-translational modification (e.g., phosphorylation) of the Cx C-terminus regulates transport to and from the plasma membrane (Lampe P D, et al. (2000) Arch Biochem Biophys. 384:205-215) and the N-terminus contributes to channel gating (Lee H J, et al. (2020) Science advances. 6:eaba4996). Motifs in and near the TM domains affect connexin oligomerization and gap junction or hemichannel conductance (Hu X, et al. (2006) Biophysical journal. 90:140-150). The ELs primarily function to impart hemichannel docking specificity (Koval M, et al. (2014) Febs Letters. 588:1193-1204), though EL1 can also contribute to channel properties including permeability and conductance (Oh S, et al. (1999) The J Gen Physiol. 114(3):339-364; Trexler E B, et al. (2000) Biophys J. 79:3036-3051; Bai D L, et al. (2014) Biochem J. 458:1-10). Thus, the primary differentiating features of connexins are trafficking and assembly, oligomerization specificity, docking specificity and permeability or conductance.

The presence of multiple unique Cx isoforms throughout the body diversifies gap junction intercellular communication. For example, all Cxs form homotypic gap junctions (two hemichannels of one isoform (e.g., Cx36/Cx36 gap junctions) between two neurons (Deans M R, et al. (2002) Neuron. 36:703-712). But most isoforms can also form heterotypic channels; that it, two hemichannels of different isoforms (e.g., Cx46/Cx50 gap junctions in the lens (Lampe P D, et al. (2000) Arch Biochem Biophys. 384:205-215)) with compatibility being dictated by motifs in the ELs. Frequently, because each isoform has slightly variable permeability and conductivity, heterotypic channels exhibit a preferred ionic directional flow (rectification). Additionally, within cells in which more than one Cx is expressed, multiple isoforms may be mixed into single hemichannels, called hetero-oligomerized or heteromeric hemichannels (e.g., cardiac Cx43/Cx45 hemichannels (Martinez A D, et al. (2002) Circ Res. 90:1100-1107), with compatibility dictated by motifs within and adjacent to TM regions (Smith T D, et al. (2012) J Membr Biol. 245(5-6):221-230; Koval M, et al. (2014) Febs Letters. 588:1193-1204).

Another important feature of gap junction intercellular communication centers on the lifetime of connexin proteins at the plasma membrane being relatively short with half-lives of ˜1-5 hrs (Laird D W, et al. (1991) Biochem J. 273(Pt 1):67-72; Fallon R F, et al. (1981) J Cell Biol. 90:521-526; Laing J G, et al. (1995) J Biol Chem. 270:26399-26403; Beardslee M A, et al. (1998) Circ Res. 83:629-635). Given the critical utility of Cxs throughout the body, this short half-life indicates that Cxs are maintained in a constant flux of biosynthesis and degradation, which allows a cell to rapidly up-regulate or down-regulate gap junction intercellular communication in response to physiological needs. Importantly, once hemichannels dock, they are virtually inseparable under physiological conditions (Ghoshroy S, et al. (1995) J Membr Biol. 146(1):15-28; Goodenough D A, et al. (1974) J Cell Biol. 61:575-590). Thus, down-regulation of gap junction intercellular communication is achieved by functional gap junction internalization. Specifically, gap junctions are turned over from the plasma membrane via a unique clathrin/dynamin-dependent internalization process (Piehl M, et al. (2007) Mol Biol Cell. 18:337-347; Gumpert A M, et al. (2008) FEBS Lett. 582:2887-2892; Nickel B M, et al. (2008) Biochem Biophys Res Commun. 374:679-682) that results in the internalization of portions of or entire gap junction plaques in the form of double-bilayer vesicular structures, termed annular gap junctions or, more recently, connexosomes (Larsen W J, et al. (1979) J Cell Biol. 83(3):576-587; Mazet F, et al. (1985) Circ Res. 56:195-204; Jordan K, et al. (2001) J Cell Sci. 114:763-773). Connexosomes contain fully-docked gap junctions that are either degraded (Piehl M, et al. (2007) Mol Biol Cell. 18:337-347) or recycled and transported back to the plasma membrane (Boassa D, et al. (2010) Traffic. 11:1471-1486; Vanderpuye O A, et al. (2016) Cell Biol Int. 40:387-396; Bell C L, et al. (2019a) Int J Mol Sci. 20(1):44).

TABLE 1
Genetic Disorders Caused by Human Connexin Mutations.

| Gene | Chrom. | Protein | Disorder or Disorders | OMIM |
|---|---|---|---|---|
| GJA1 | 6q22.31 | Cx43 | Craniometaphyseal dysplasia, autosomal recessive | 218400 |
| | | | Erythrokeratodermia variabilis et progressive | 133200 |
| | | | Oculodentodigital dysplasia | 164200 |
| | | | Oculodentodigital dysplasia, autosomal recessive | 257850 |
| | | | Palmoplantar keratoderma with congenital alopecia | 104100 |
| | | | Syndactyly, type III | 186100 |
| GJA3 | 13q12.11 | Cx46 | Cataract | 601885 |
| GJA4 | 1p34.3 | Cx37 | | |
| GJA5 | 1q21.2 | Cx40 | Atrial fibrillation, familial, 11 | 614049 |
| | | | Atrial standstill, digenic (GJA5/SCN5A) | 108770 |
| GJA8 | 1q21.2 | Cx50 | Cataract | 116200 |
| GJA9 | 1p34.3 | Cx59 | | |
| GJA10 | 6q15 | Cx62 | | |
| GJB1 | Xq13.1 | Cx32 | Charcot-Marie-Tooth neuropathy, X-linked 1 | 302800 |
| GJB2 | 13q12.11 | Cx26 | Bart-Pumphrey syndrome | 149200 |
| | | | Deafness, autosomal dominant 3A | 601544 |
| | | | Deafness, autosomal recessive 1A | 220290 602540 |
| | | | Hystrix-like ichthyosis with deafness | 148210 |
| | | | Keratitis-ichthyosis-deafness syndrome | 148350 |
| | | | Keratoderma, palmoplantar, with deafness | 124500 |
| | | | Vohwinkel syndrome | |
| | | | Porokeratotic eccrine ostial and dermal duct nevus | |
| GJB3 | 1p34.3 | Cx31 | Deafness, autosomal dominant 2B | 612644 |
| | | | Deafness, digenic, (GJB2/GJB3) | 220290 |
| | | | Erythrokeratodermia variabilis et progressiva | 133200 |
| GJB4 | 1p34.3 | Cx30.3 | Erythrokeratodermia variabilis et progressiva | 133200 |
| GJB5 | 1p34.3 | Cx31.1 | | |
| GJB6 | 13q12.11 | Cx30 | Deafness, autosomal dominant 3B | 612643 |
| | | | Deafness, autosomal recessive 1B | 612645 |
| | | | Deafness, digenic (GJB2/GJB6) | 220290 |
| | | | Ectodermal dysplasia 2, Clouston type | 129500 |
| GJB7 | 6q14.3-q15 | Cx25 | | |
| GJC1 | 17q21.31 | Cx45 | | |
| GJC2 | 1q42.13 | Cx47 | Leukodystrophy, hypomyelinating, 2 | 608804 |
| | | | Spastic paraplegia 44, autosomal recessive | 613206 |
| | | | Lymphedema, hereditary, IC | 613480 |
| GJC3 | 7q22.1 | Cx30.2 | | |
| GJD2 | 15q14 | Cx36 | | |
| GJD3 | 17q21.2 | Cx31.9 | | |
| GJD4 | 10p11.21 | Cx40.1 | | |
| GJE1 | 6q24.1 | Cx23 | | |

Despite the numerous and broad implications of connexin isoforms in human physiology and disease (see Table 1 above), available tools for discerning key attributes such as hemichannel docking and permeability in vitro are currently limited. The most readily available methods for detecting activity (i) often rely on dye transfer (e.g., GAP-FRAP, scrape loading, microinjection, etc.), which in turn depend on the permeability of channels to small molecule dyes such as calcein, carboxyfluorescein, Lucifer yellow, and ethidium bromide, and (ii) use the transfer of these dyes between adjacent cells as indicators of functional gap junctions (Abbaci M, et al. (2008) Biotechniques. 45:33-62). However, very few dyes have been established as gap junction permeant and such dyes are generally connexin isoform, isoform species, and/or pH specific (Elfgang C, et al. (1995) J Cell Biol. 129:805-81). The highest precision and therefore “gold-standard” method for evaluating connexin activity is whole-cell, dual patch clamp measurement (Veenstra R D, et al. (1986) Science. 233:972-974; White T, et al. (1995) Mol Biol Cell. 6(4):459-470), which can quantitatively characterize the electrical coupling (conductance) between two cells mediated by gap junctions with high sensitivity. But this is a very low-throughput, labor intensive, and expensive technique. Thus, what it desperately needed is a higher throughput, universal method for detection/determination of connexin interactions would contribute more comprehensive understanding of connexin biology.

### B. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5 and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, geriatric, adult, adolescent, and child subjects, as well as fetuses, whether male or female, are intended to be covered. In an aspect, a subject can be a human subject. In an aspect, a subject can have a disease or disorder characterized by gap junction dysfunction or misfunction. In an aspect, a subject can be suspected of having a disease or disorder characterized by gap junction dysfunction or misfunction. In an aspect, a subject can have a disease or disorder characterized by a cellular circuit dysfunction or misfunction. In an aspect, a subject can have a disease or disorder characterized by asynchrony or electrical asynchrony.

As used herein, the term “diagnosed” means having been subjected to an examination by a person of skill, for example, a physician, and found to have a condition that can be diagnosed or treated by one or more of the disclosed compositions or by one or more of the disclosed methods. For example, “diagnosed with a disease or disorder characterized by gap junction dysfunction and/or malfunction” means having been subjected to an examination by a person of skill, for example, a physician, and found to have a condition that can be treated by one or more of the disclosed compositions or by one or more of the disclosed methods. For example, “suspected of having a disease or disorder characterized by gap junction dysfunction and/or malfunction” can mean having been subjected to an examination by a person of skill, for example, a physician, and found to have a condition that can likely be treated by one or more of the disclosed compositions or by one or more of the disclosed methods. In an aspect, an examination can be physical, can involve various tests (e.g., blood tests, genotyping, biopsies, etc.), diagnostic evaluations (e.g., X-ray, CT scan, etc.), and assays (e.g., enzymatic assay), or a combination thereof. In an aspect, an examination can be objective and/or subjective.

A “patient” refers can refer to a subject afflicted with a disease or disorder (e.g., gap junction dysfunction and/or malfunction and/or cellular asynchrony and/or electrical asynchrony). In an aspect, a patient can be in the military or can be a veteran. In an aspect, a patient can refer to a subject that has been diagnosed with or is suspected of having a disease or disorder characterized by gap junction dysfunction and/or malfunction. In an aspect, a patient can refer to a subject that has been diagnosed with or is suspected of having a disease or disorder and is seeking treatment or receiving treatment for a disease or disorder (such as a disease or disorder characterized by gap junction dysfunction and/or malfunction). In an aspect, a “patient” can refer to a subject afflicted with a disease or disorder characterized by gap junction dysfunction and/or malfunction. In an aspect, a patient can refer to a subject that has been diagnosed with or is suspected of having a disease or disorder a disease or disorder characterized by gap junction dysfunction and/or malfunction. In an aspect, a patient can refer to a subject that has been diagnosed with or is suspected of having a disease or disorder and is seeking treatment or receiving treatment for a disease or disorder (such as a disease or disorder characterized by gap junction dysfunction and/or malfunction).

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, the phrase “identified to be in need of treatment,” or the like, refers to selection of a subject based upon need for treatment of a disease or disorder characterized by gap junction dysfunction and/or malfunction and/or cellular asynchrony and/or electrical asynchrony. For example, a subject can be identified as having a need for treatment based upon an earlier diagnosis by a person of skill and thereafter subjected to treatment for a disease or disorder characterized by gap junction dysfunction and/or malfunction and/or cellular asynchrony and/or electrical asynchrony. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. In an aspect, “inhibiting” can refer to diminishing the intensity, the duration, the amount, or a combination thereof of symptoms, complications, issues due to a subject's gap junction dysfunction and/or malfunction. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder characterized by gap junction dysfunction and/or malfunction and/or cellular asynchrony and/or electrical asynchrony) or to the level prior to the onset of a disease or disorder characterized by gap junction dysfunction and/or malfunction and/or cellular asynchrony and/or electrical asynchrony. Thus, in an aspect, the inhibition or reduction can be a 10%, 20%, 30%, 40%, 50%, 60%, 70%, 80%, 90%, 100%, or any amount of reduction in between as compared to native or control levels or to the subject's level prior to the onset of gap junction dysfunction and/or malfunction and/or cellular asynchrony and/or electrical asynchrony. In an aspect, the inhibition or reduction can be 10-20%, 20-30%, 30-40%, 40-50%, 50-60%, 60-70%, 70-80%, 80-90%, or 90-100% as compared to native or control levels or to the subject's level prior to the onset of gap junction dysfunction and/or malfunction or a disease or disorder characterized by gap junction dysfunction and/or malfunction and/or cellular asynchrony and/or electrical asynchrony. In an aspect, the inhibition or reduction can be 0-25%, 25-50%, 50-75%, or 75-100% as compared to native or control levels or to the subject's level prior to the onset to the onset of gap junction dysfunction and/or malfunction and/or cellular asynchrony and/or electrical asynchrony.

The words “treat” or “treating” or “treatment” include palliative treatment, that is, treatment designed for the relief of symptoms rather than the curing of a disease or disorder characterized by gap junction dysfunction and/or malfunction and/or cellular asynchrony and/or electrical asynchrony; preventative treatment, that is, treatment directed to minimizing or partially or completely inhibiting the development of a disease or disorder characterized by gap junction dysfunction and/or malfunction and/or cellular asynchrony and/or electrical asynchrony; and supportive treatment, that is, treatment employed to supplement another specific therapy directed toward the improvement of a disease or disorder characterized by gap junction dysfunction and/or malfunction and/or cellular asynchrony and/or electrical asynchrony. In an aspect, the terms cover any treatment of a subject, including a mammal (e.g., a human), and includes: (i) preventing the undesired physiological change and/or pathological condition from occurring in a subject that can be predisposed to a disease or disorder characterized by gap junction dysfunction and/or malfunction but has not yet been diagnosed as having it; (ii) inhibiting the physiological change and/or pathological condition (a disease or disorder characterized by gap junction dysfunction and/or malfunction and/or cellular asynchrony and/or electrical asynchrony); or (iii) relieving the physiological change and/or pathological condition, i.e., causing regression of a disease or disorder characterized by gap junction dysfunction and/or malfunction and/or cellular asynchrony and/or electrical asynchrony. For example, in an aspect, treating a disease or disorder can reduce the severity of an established a disease or disorder in a subject by 1%-100% as compared to a control (such as, for example, an individual not having a disease or disorder characterized by gap junction dysfunction and/or malfunction and/or cellular asynchrony and/or electrical asynchrony). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or a disorder or a condition (such as a disease or disorder characterized by gap junction dysfunction and/or malfunction). For example, treating a disease or a disorder can reduce one or more symptoms of a disease or disorder in a subject by 1%-100% as compared to a control (such as, for example, an individual not having a disease or disorder characterized by gap junction dysfunction and/or malfunction and/or cellular asynchrony and/or electrical asynchrony). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or a disorder or a condition (e.g., gap junction dysfunction and/or malfunction and/or cellular asynchrony and/or electrical asynchrony). It is understood that treatment does not necessarily refer to a cure or complete ablation or eradication of a disease or disorder characterized by gap junction dysfunction and/or malfunction and/or cellular asynchrony and/or electrical asynchrony. However, in an aspect, treatment can refer to a cure or complete ablation or eradication of a disease or a disorder or a condition (such as gap junction dysfunction and/or malfunction and/or cellular asynchrony and/or electrical asynchrony).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing gap junction dysfunction and/or malfunction and/or cellular asynchrony and/or electrical asynchrony or the worsening of gap junction dysfunction and/or malfunction and/or cellular asynchrony and/or electrical asynchrony is intended. The words “prevent” and “preventing” and “prevention” also refer to prophylactic or preventative measures for protecting or precluding a subject (e.g., an individual) not having gap junction dysfunction and/or malfunction or a given gap junction dysfunction and/or malfunction and/or cellular asynchrony and/or electrical asynchrony related complication from progressing to that complication.

As used herein, the terms “administering” and “administration” refer to any method of providing one or more of the disclosed compositions (such as, for example, a disclosed viral vector). Such methods are well-known to those skilled in the art and include, but are not limited to, the following: oral administration, transdermal administration, administration by inhalation, nasal administration, topical administration, in utero administration, intrahepatic administration, intravaginal administration, epidural administration (such as epidural injection), intracerebroventricular (ICV) administration, ophthalmic administration, intraaural administration, depot administration, topical (skin) administration, otic administration, intra-articular (such as joint or vertebrate injection), intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration of a disclosed composition, a disclosed viral vector, a disclosed pharmaceutical formulation, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route. In an aspect, a disclosed composition, a disclosed viral vector, a disclosed pharmaceutical formulation, or any combination thereof can be concurrently and/or serially administered to a subject via multiple routes of administration. For example, in an aspect, administering a disclosed composition, a disclosed viral vector, a disclosed pharmaceutical formulation, or any combination thereof can comprise intravenous administration and intra-cistern magna (ICM) administration. In an aspect, administering a disclosed composition, a disclosed viral vector, a disclosed pharmaceutical formulation, or any combination thereof can comprise IV administration and intrathecal (ITH) administration. Various combinations of administration are known to the skilled person.

By “determining the amount” is meant both an absolute quantification of a particular analyte (e.g., fluorescence) or a determination of the relative abundance of a particular analyte (e.g., a dual-labeled connexosome). The phrase includes both direct or indirect measurements of abundance or both. In an aspect, determining the amount can refer to measuring the electrical activity and/or electrical performance of a targeted circuit.

As used herein, “modifying the method” can comprise modifying or changing one or more features or aspects of one or more steps of a disclosed method. In an aspect, a method can be altered by changing the amount of one or more of the disclosed compositions (e.g., a disclosed viral vector) used in a disclosed method, or by changing the frequency of administration of one or more disclosed compositions (e.g., a disclosed viral vector) in a disclosed method, by changing the duration of time that one or more disclosed compositions (e.g., a disclosed viral vector) is administered in a disclosed method, or by substituting for one or more of the disclosed components and/or reagents with a similar or equivalent component and/or reagent.

As used herein, “concurrently” means (1) simultaneously in time, or (2) at different times during a common treatment schedule.

In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

As used herein, “heterotypic binding” or “heterotopically binding” refers to the binding of hemichannels comprising differing connexins. For example, a hemichannel comprising Cx35 can heterotopically bind to a hemichannel comprising Cx34.7.

As used herein, “homotypic binding” or “homotypically binding” or “homotypic docking” or “homotypically docking” refers to the binding of hemichannels comprising identical connexins. For example, a hemichannel comprising Cx35 homotypically binds to a hemichannel comprising Cx35 while a hemichannel comprising Cx34.7 homotypically binds to a hemichannel comprising Cx34.7.

The term “contacting” as used herein refers to bringing one or more of the disclosed compositions (e.g., a disclosed viral vector) together with a target area or intended target area (e.g., a population of cells) in such a manner that the disclosed compositions can exert an effect on the intended target or targeted area either directly or indirectly. A target area or intended target area can be one or more cells and/or one or more brain regions and/or one or more cells and/or tissues experiencing asynchrony and/or gap junction dysfunction, or any combination thereof. In an aspect, a target area or intended target area can be any cell or any organ infected by a disease or disorder (such as a cell or organ characterized by gap junction dysfunction and/or malfunction and/or cellular asynchrony and/or electrical asynchrony). In an aspect, a target area or intended target area can be any organ, tissue, or cells that are affected by a disease or disorder characterized by gap junction dysfunction and/or malfunction and/or cellular asynchrony and/or electrical asynchrony.

As used herein, “determining” can refer to measuring or ascertaining the presence and severity of a disease or disorder, such as, for example, characterized by gap junction dysfunction and/or malfunction and/or cellular asynchrony and/or electrical asynchrony. “Determining” can refer to measuring or ascertaining fluorescence. “Determining” can refer to measuring or ascertaining gap junction formation, or measuring or ascertaining docking interactions between connexins, or measuring or ascertaining gap junction hemichannel docking. “Determining” can refer to measuring or ascertaining cellular asynchrony and/or electrical asynchrony or measuring or ascertaining cellular synchrony and/or electrical synchrony.

Methods and techniques used to determine the presence and/or severity of a disease or disorder characterized by gap junction dysfunction and/or malfunction and/or cellular asynchrony and/or electrical asynchrony are typically known to the medical arts. For example, the art is familiar with the ways to identify and/or diagnose the presence, severity, or both of a disease or disorder characterized by gap junction dysfunction and/or malfunction and/or cellular asynchrony and/or electrical asynchrony. Methods can be based on objective and/or subjective means.

As used herein, “effective amount” and “amount effective” can refer to an amount that is sufficient to achieve the desired result such as, for example, the treatment and/or prevention of a disease or disorder characterized by gap junction dysfunction and/or malfunction and/or cellular asynchrony and/or electrical asynchrony. As used herein, the terms “effective amount” and “amount effective” can refer to an amount that is sufficient to achieve the desired an effect on an undesired condition (e.g., a disease or disorder characterized by gap junction dysfunction and/or malfunction and/or cellular asynchrony and/or electrical asynchrony). For example, a “therapeutically effective amount” refers to an amount that is sufficient to achieve the desired therapeutic result or to have an effect on undesired symptoms, but is generally insufficient to cause adverse side effects.

In an aspect, “therapeutically effective amount” means an amount of the disclosed composition that (i) treats a disease or disorder characterized by gap junction dysfunction and/or malfunction and/or cellular asynchrony and/or electrical asynchrony, (ii) attenuates, ameliorates, or eliminates one or more symptoms associated with a a disease or disorder characterized by gap junction dysfunction and/or malfunction and/or cellular asynchrony and/or electrical asynchrony, or (iii) delays the onset of one or more symptoms of a disease or disorder characterized by gap junction dysfunction and/or malfunction and/or cellular asynchrony and/or electrical asynchrony. The specific therapeutically effective dose level for any particular patient will depend upon a variety of factors including the disease or disorder characterized by gap junction dysfunction and/or malfunction and/or cellular asynchrony and/or electrical asynchronybeing treated; the disclosed compositions employed; the disclosed methods employed; the age, body weight, general health, sex and diet of the patient; the time of administration; the route of administration; the rate of excretion of the disclosed compositions employed; the duration of the treatment; drugs used in combination or coincidental with the disclosed compositions employed, and other like factors well known in the medical arts. For example, it is well within the skill of the art to start doses of the disclosed compositions at levels lower than those required to achieve the desired therapeutic effect and to gradually increase the dosage until the desired effect is achieved. If desired, then the effective daily dose can be divided into multiple doses for purposes of administration. Consequently, a single dose of the disclosed compositions, disclosed viral vectors, disclosed pharmaceutical formulations, disclosed therapeutic agents, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a sign or symptom associated with a disease or disorder characterized by gap junction dysfunction and/or malfunction and/or cellular asynchrony and/or electrical asynchrony.

As used herein, the term “package insert” is used to refer to instructions customarily included in commercial packages of therapeutic products, that contain information about the indications, usage, dosage, administration, contraindications and/or warnings concerning the use of such therapeutic products.

Disclosed are the components to be used to prepare the disclosed compositions, disclosed viral vectors, disclosed pharmaceutical formulations, disclosed therapeutic agents, or a combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### C. Review of FETCH

In an aspect, FETCH is comprehensively described in Int'l. Application No. PCT/US2022/032087 (filing date of Jun. 3, 2022), which is incorporated herein by reference in its entirety for teaching FETCH). Briefly, FETCH can comprises interrogating the docking interactions between connexins by performing flow enabled tracking of connexosomes in HEK293T cells (FETCH).

Briefly, in an aspect, performing FETCH can comprises (i) generating fluorescent connexosomes, and (ii) using flow cytometry to track the fluorescent connexosomes in one or more cell samples. In an aspect, generating one or more fluorescent connexosomes can comprise transfecting a first population of cells with a first fluorescently-labeled, C-terminally fused connexin construct; and transfecting a second population of cells with a second fluorescently-labeled, C-terminally fused connexin construct.

In an aspect, a disclosed method of performing FETCH can further comprise incubating the transfected populations of cells. In an aspect, incubating can comprise less than 10 hrs or more than 24 hrs. In an aspect, a disclosed method can further comprise trypsinizing the incubated and transfected population of cells. Trypsinization, methods of trypsinization, and agents for trypsinizing are known to the art. In an aspect, a disclosed method of performing FETCH can further comprise incubating the trypsinized first population of cells with the trypsinized second population of cells.

In an aspect of a disclosed method, incubating the combined populations of cells can comprise about 10 hours to about 30 hours. In an aspect, incubating the combined populations of cells can comprise about 20 hrs. In an aspect of a disclosed method, incubating the combined populations of cells can continue to hyperdensity and/or over-confluency. In an aspect, a disclosed method of performing FETCH can comprise trypsinizing the incubated combined population of cells, resuspending the trypsinized cells, and fixing the resuspended cells. In an aspect, resuspending the trypsinized cells can comprise PBS and DNase. In an aspect of a disclosed method, fixing the resuspended cells can comprise paraformaldehyde. Methods of fixation and agents for fixation are known to the art.

In an aspect of a disclosed method of performing FETCH, during flow cytometry, the cells can be analyzed in two selection gates prior to fluorescence evaluation. In an aspect, analyzing the cells in two selection gates can comprises (i) identifying cells by evaluating sample forward vs. side scatter area; and (ii) identifying single cells by evaluating cells that maintained a linear correlation of forward scatter height to forward scatter area. In an aspect, incubating the combined populations of cells can comprise further comprising generating one or more fluorescently-labeled, C-terminally fused connexin constructs.

In an aspect, flow cytometry can generate a fluorescent profile for each cell sample or each population of cells. In an aspect, a disclosed fluorescence profile can comprise 4 quadrants. In an aspect, disclosed quadrants can comprise Q1, Q2, Q3, and Q4. In an aspect, a disclosed Q1 can represent a population of cells with the first fluorescently-labeled, C-terminally fused connexin construct. In an aspect, a disclosed Q2 can represent a population of cells expressing both the first fluorescently-labeled, C-terminally fused connexin construct and the second fluorescently-labeled C-terminally fused connexin construct. In an aspect, a disclosed Q3 can represent a population of cells with the second fluorescently-labeled C-terminally fused connexin construct. In an aspect, a disclosed Q4 can represent untransfected cells in either the first population of cells or the second populations of cells.

In an aspect, a disclosed method can further comprise establishing a FETCH score. In an aspect, a disclosed FETCH score can be a positive FETCH score or a negative FETCH score. In an aspect, a disclosed positive FETCH score can indicate a docking event. In an aspect, a disclosed negative FETCH score can indicate a non-docking event. In an aspect, a disclosed negative FETCH score can indicate dysfunctional gap junction intercellular communication. In an aspect, a disclosed FETCH score can comprise the proportion of dual colored-cells compared to fluorescent cells. In an aspect, a disclosed proportion can comprise Q2/(Q1+Q2+Q3). In an aspect, a disclosed method can comprise quantifying the fluorescence exchange between cells mediated by connexosomes.

In an aspect, a disclosed method can comprise confirming the relationship between the fluorescence exchange phenotype and the connexosome formation. In an aspect, confirming the relationship between the fluorescence exchange phenotype and connexosome formation can comprise using fluorescence-activated cell sorting (FACS) to collect cells from Q2 for microscopy analysis. In an aspect, FACS can confirm the formation of dual-labeled connexosomes. In an aspect, a disclosed method of interrogating the docking interactions between connexins can identify homotypic docking of hemichannels. In an aspect, a disclosed method can identify homotypic docking of hemichannels. In an aspect, disclosed homotypic docking can comprise docking of two hemichannels having a single connexin isoform. In an aspect, a disclosed method can identify heterotypic docking of hemichannels. In an aspect, a disclosed heterotypic docking can comprise docking of two hemichannels having a different connexin isoform.

In an aspect, a disclosed method of performing FETCH can further comprise performing whole-cell, dual-patch clamp analysis on a disclosed combined population of cells. In an aspect, a disclosed method can identify variation in the expression level of one or more disclosed connexin constructs. In an aspect, a disclosed method can identify variation in trafficking of one or more disclosed connexin constructs. In an aspect, a disclosed method can identify variation in stability of one or more disclosed connexin constructs. In an aspect, a disclosed method of interrogating the docking interactions between connexins can identify variation in the turnover rate of one or more disclosed connexin constructs. In an aspect, a disclosed method does not quantify hemichannel binding affinity.

In an aspect, a disclosed FETCH score can reflect variation in (i) the expression level of the connexin constructs, (ii) the trafficking of the connexin constructs, (iii) the stability of the connexin constructs, and/or (iv) the turnover rate of the connexin constructs. In an aspect, a disclosed method of interrogating the docking interactions between connexins can be a downstream indicator of connexin hemichannel docking compatibility. In an aspect, a disclosed method can be scaled for high-throughput analysis.

In an aspect, a disclosed method can further comprise treating a subject having a disease or disorder characterized by gap junction dysfunction and/or malfunction. In an aspect, gap junction dysfunction and/or malfunction can be confirmed by a disclosed method. In an aspect, a disclosed method can further comprise evaluating gap junction formation. In an aspect, a disclosed method can further comprise high-throughput quantification of gap junction hemichannel docking. In an aspect, a disclosed method of interrogating the docking interactions can further comprise evaluating gap junction formation and high-throughput quantification of gap junction hemichannel docking.

In an aspect, a disclosed method can use a disclosed automated pipeline that can receive and process FETCH data. A disclosed automated pipeline is described in Int'l. App. No. PCT/US2022/032087.

### D. Compositions for Use in the Disclosed Methods

### 1. Connexin Nucleic Acid Molecules

Disclosed herein is an isolated nucleic acid molecule comprising a nucleic acid sequence encoding a designer connexin protein.

In an aspect, a disclosed encoded designer connexin protein can be derived from a non-mammalian species. In an aspect, a disclosed encoded designer connexin protein can be docking incompetent with a connexin endogenously expressed in the mammalian central nervous system. In an aspect, docking incompetent can mean not capable of docking with a connexin naturally expressed in a mammalian CNS. In an aspect, a disclosed encoded designer connexin protein can be capable of heterotypic binding or can heterotopically bind with another designer connexin protein. In an aspect, a disclosed encoded designer connexin protein can be not capable of homotypic binding or homotypic docking or cannot homotypically bind or homotypically dock to the identical connexin protein.

In an aspect, a disclosed encoded designer connexin protein can comprise Cx34.7_{M1} or Cx35_{M1}. In an aspect, Cx34.7_{M1} can comprise the Cx34.7 of Morone americana having a substitution at encoded amino acid 214 and at encoded amino acid 223. In an aspect, a disclosed substitution at encoded amino acid 214 can comprise a glutamic acid for a lysine (E214K). In an aspect, a disclosed substitution at encoded amino acid 223 can comprise a glutamic acid for a lysine (E223K).

In an aspect, wild-type Cx34.7 in Morone americana can comprise 306 amino acids. In an aspect, wild-type Cx34.7 in Morone americana can comprise the sequence set forth in GenBank Accession No. AAC31885.1 or a fragment thereof. In an aspect, the nucleotide sequence encoding wild-type Cx34.7 in Morone americana can comprise about 1871 bp. In an aspect, the nucleotide sequence encoding wild-type Cx34.7 in Morone americana can comprise the sequence set forth in GenBank Accession No. AF059184.1.

In an aspect, wild-type Cx35 in Morone americana can comprise 304 amino acids. In an aspect, wild-type Cx35 in Morone americana can comprise the sequence set forth in GenBank Accession No. AAC31884.1 or a fragment thereof. In an aspect, the nucleotide sequence encoding wild-type Cx35 in Morone americana can comprise about 1320 bp. In an aspect, the nucleotide sequence encoding wild-type Cx35 in Morone americana can comprise the sequence set forth in GenBank Accession No. AF059183.1.

In an aspect, Cx35M1 can comprise the Cx35 of Morone americana having a substitution at encoded amino acid 221. In an aspect, a disclosed substitution at encoded amino acid 221 can comprise a lysine for a glutamic acid (K221E).

In an aspect, Cx34.7_{M1} can heterotypically bind only to Cx35M1. In an aspect, Cx35M1 can heterotypically bind only to Cx34.7M1.

In an aspect, a disclosed encoded designer connexin protein can comprise the sequence set forth in SEQ ID NO:19 with a substitution at encoded amino acid 214 and at encoded amino acid 223. In an aspect, a disclosed encoded designer connexin protein can comprise the sequence set forth in SEQ ID NO:20 with a substitution at encoded amino acid 221. In an aspect, a disclosed nucleic acid sequence encoding the designer connexin protein can comprise the sequence set forth in any one of SEQ ID NO:23, SEQ ID NO:24, SEQ ID NO:25, or SEQ ID NO:26 or a fragment thereof.

In an aspect, a disclosed nucleic acid sequence encoding a designer connexin protein can be codon-optimized for expression in a mammalian cell or a human cell. In an aspect, a disclosed nucleic acid sequence encoding a designer connexin protein can be CpG-free or CpG-depleted.

In an aspect, a disclosed isolated nucleic acid molecule can comprise a nucleic acid sequence encoding a carboxy-terminal fluorescent label and/or fluorescent tag.

In an aspect, a disclosed isolated nucleic acid molecule can comprise a nucleic acid sequence encoding an amino-terminal fluorescent label and/or fluorescent tag.

In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art. In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed isolated nucleic acid sequence encoding a designer connexin protein can restore the functionality and/or synchrony of a circuit (such as a neural circuit or a neuromuscular circuit).

In an aspect, a disclosed isolated nucleic acid sequence encoding a designer connexin protein can be validated and/or characterized using FETCH. In an aspect, a disclosed isolated nucleic acid sequence encoding a designer connexin protein can be validated and/or characterized using an animal model such as mice and/or C. elegans

In an aspect, a disclosed isolated nucleic acid sequence encoding a designer connexin protein can be packaged in a viral vector (as discussed infra) or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, following expression, a disclosed isolated nucleic acid molecule comprising a nucleic acid sequence encoding a designer connexin protein can achieve context-precise circuit-editing characterized by a spatiotemporal specificity.

In an aspect, following expression, a disclosed isolated nucleic acid molecule comprising a nucleic acid sequence encoding a designer connexin protein can synchronize electrical activity in a cellular circuit.

Disclosed herein is an isolated nucleic acid molecule comprising a nucleic acid sequence encoding a designer Cx34.7 connexin protein.

In an aspect, a disclosed designer Cx34.7 connexin protein can comprise the Cx34.7 of Morone americana having a substitution at encoded amino acid 214 and at encoded amino acid 223. In an aspect, a disclosed substitution at encoded amino acid 214 can comprise a glutamic acid for a lysine (E214K). In an aspect, a disclosed substitution at encoded amino acid 223 can comprise a glutamic acid for a lysine (E223K).

In an aspect, a disclosed encoded designer Cx34.7 connexin protein can comprise the sequence set forth in SEQ ID NO:19 with a substitution at encoded amino acid 214 and at encoded amino acid 223. In an aspect, a disclosed nucleic acid sequence encoding the designer Cx34.7 connexin protein can comprise the sequence set forth in SEQ ID NO:23-SEQ ID NO:24 or a fragment thereof.

In an aspect, a disclosed nucleic acid sequence encoding a designer Cx34.7 connexin protein can be codon-optimized for expression in a human cell or a human cell. In an aspect, a disclosed nucleic acid sequence encoding a designer Cx34.7 connexin protein can be CpG-free or CpG-depleted.

In an aspect, a disclosed nucleic acid sequence encoding a designer Cx34.7 connexin protein can comprise a nucleic acid sequence encoding a carboxy-terminal fluorescent label and/or fluorescent tag. In an aspect, a disclosed nucleic acid sequence encoding a designer Cx34.7 connexin protein can comprise a nucleic acid sequence encoding an amino-terminal fluorescent label and/or fluorescent tag. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art. In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed isolated nucleic acid sequence encoding a designer Cx34.7 connexin protein can restore the functionality and/or synchrony of a circuit (such as a neural circuit or a neuromuscular circuit).

In an aspect, a disclosed isolated nucleic acid sequence encoding a designer Cx34.7 connexin protein can be validated and/or characterized using FETCH. In an aspect, a disclosed isolated nucleic acid sequence encoding a designer Cx34.7 connexin protein can be validated and/or characterized using an animal model such as mice and/or C. elegans

In an aspect, a disclosed isolated nucleic acid sequence encoding a designer Cx34.7 connexin protein can be packaged in a viral vector (as discussed infra) or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, following expression, a disclosed isolated nucleic acid sequence encoding a designer Cx34.7 connexin protein can achieve context-precise circuit-editing characterized by a spatiotemporal specificity

In an aspect, following expression, a disclosed isolated nucleic acid molecule comprising a nucleic acid sequence encoding a designer Cx34.7 connexin protein can synchronize electrical activity in a cellular circuit.

Disclosed herein is an isolated nucleic acid molecule, comprising: a nucleic acid sequence encoding a designer Cx35 connexin protein.

In an aspect, Cx35_{M1} can comprise the Cx35 of Morone americana having a substitution at encoded amino acid 221. In an aspect, a disclosed substitution at encoded amino acid 221 can comprise a lysine for a glutamic acid (K221E).

In an aspect, a disclosed encoded designer Cx35 connexin protein can comprise the comprises the sequence set forth in SEQ ID NO:20 with a substitution at encoded amino acid 221. or a fragment thereof. In an aspect, a disclosed nucleic acid sequence encoding the designer Cx35 connexin protein can comprise the sequence set forth in SEQ ID NO:25-SEQ ID NO:26 or a fragment thereof.

In an aspect, a disclosed nucleic acid sequence encoding a designer Cx35 connexin protein can be codon-optimized for expression in a mammalian cell or a human cell. In an aspect, a disclosed nucleic acid sequence encoding a designer Cx35 connexin protein can be CpG-free or CpG-depleted.

In an aspect, a disclosed nucleic acid sequence encoding a designer Cx35 connexin protein can comprise a nucleic acid sequence encoding a carboxy-terminal fluorescent label and/or fluorescent tag. In an aspect, a disclosed nucleic acid sequence encoding a designer Cx35 connexin protein can comprise a nucleic acid sequence encoding an amino-terminal fluorescent label and/or fluorescent tag. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed isolated nucleic acid sequence encoding a designer Cx35 connexin protein can restore the functionality and/or synchrony of a circuit (such as a neural circuit or a neuromuscular circuit).

In an aspect, a disclosed isolated nucleic acid sequence encoding a designer Cx35 connexin protein can be validated and/or characterized using FETCH. In an aspect, a disclosed isolated nucleic acid sequence encoding a designer Cx35 connexin protein can be validated and/or characterized using an animal model such as mice and/or C. elegans

In an aspect, a disclosed isolated nucleic acid sequence encoding a designer Cx35 connexin protein can be packaged in a viral vector (as discussed infra) or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, following expression, a disclosed isolated nucleic acid sequence encoding a designer Cx35 connexin protein can achieve context-precise circuit-editing characterized by a spatiotemporal specificity.

In an aspect, following expression, a disclosed isolated nucleic acid molecule comprising a nucleic acid sequence encoding a designer Cx35 connexin protein can synchronize electrical activity in a cellular circuit.

### 2. Designer Connexin Proteins

Disclosed herein is a designer connexin protein.

In an aspect, a disclosed designer connexin protein can be derived from a non-mammalian species. In an aspect, a disclosed designer connexin protein can be docking incompetent with a connexin endogenously expressed in the mammalian central nervous system. In an aspect, docking incompetent can mean not capable of docking with a connexin naturally expressed in a mammalian CNS.

In an aspect, a disclosed designer connexin protein can be capable of heterotypic binding or can heterotopically bind with another designer connexin protein.

In an aspect, a disclosed designer connexin protein can be not capable of homotypic binding or homotypic docking or cannot homotypically bind to the identical connexin protein.

In an aspect, a disclosed designer connexin protein can comprise Cx34.7_{M1} or Cx35_{M1.}

In an aspect, a disclosed designer connexin protein can comprise Cx34.7_{M1} or a fragment thereof. In an aspect, Cx34.7_{M1} can heterotypically bind only to Cx35_{M1.}

In an aspect, Cx34.7_{M1} can comprise the Cx34.7 of Morone americana having a substitution at amino acid 214 and at amino acid 223. In an aspect, a disclosed substitution at amino acid 214 can comprise a glutamic acid for a lysine (E214K). In an aspect, a disclosed substitution at amino acid 223 can comprise a glutamic acid for a lysine (E223K).

In an aspect, a disclosed encoded designer Cx34.7 connexin protein can comprise the sequence set forth in SEQ ID NO:19 with a substitution at encoded amino acid 214 and at encoded amino acid 223. In an aspect, a disclosed nucleic acid sequence encoding the designer Cx34.7 connexin protein can comprise the sequence set forth in SEQ ID NO:23 or SEQ ID NO:24 or a fragment thereof.

In an aspect, wild-type Cx34.7 in Morone americana can comprise 306 amino acids. In an aspect, wild-type Cx34.7 in Morone americana can comprise the sequence set forth in GenBank Accession No. AAC31885.1 or a fragment thereof. In an aspect, the nucleotide sequence encoding wild-type Cx34.7 in Morone americana can comprise about 1871 bp. In an aspect, the nucleotide sequence encoding wild-type Cx34.7 in Morone americana can comprise the sequence set forth in GenBank Accession No. AF059184.1.

In an aspect, a disclosed designer connexin protein can comprise Cx35_{M1} or a fragment thereof. In an aspect, Cx35_{M1} can heterotypically bind only to Cx34.7_{M1}.

In an aspect, Cx35_{M1} can comprise the Cx35 of Morone americana having a substitution at amino acid 221. In an aspect, a disclosed substitution at amino acid 221 can comprise a lysine for a glutamic acid (K221E).

In an aspect, a disclosed encoded designer Cx35 connexin protein can comprise the comprises the sequence set forth in SEQ ID NO:20 with a substitution at encoded amino acid 221. or a fragment thereof. In an aspect, a disclosed nucleic acid sequence encoding the designer Cx35 connexin protein can comprise the sequence set forth in SEQ ID NO:25 or SEQ ID NO:26 or a fragment thereof.

In an aspect, wild-type Cx35 in Morone americana can comprise 304 amino acids. In an aspect, wild-type Cx35 in Morone americana can comprise the sequence set forth in GenBank Accession No. AAC31884.1 or a fragment thereof. In an aspect, the nucleotide sequence encoding wild-type Cx35 in Morone americana can comprise about 1320 bp. In an aspect, the nucleotide sequence encoding wild-type Cx35 in Morone americana can comprise the sequence set forth in GenBank Accession No. AF059183.1.

In an aspect, a disclosed nucleic acid sequence encoding a designer connexin protein (such as for example, Cx34.7_{M1} or Cx35_{M1}) can be codon-optimized for expression in a mammalian cell or a human cell.

In an aspect, a disclosed designer connexin protein can comprise a nucleic acid sequence encoding a carboxy-terminal fluorescent label and/or fluorescent tag. In an aspect, a disclosed designer connexin protein can comprise a nucleic acid sequence encoding an amino-terminal fluorescent label and/or fluorescent tag. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art. In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed designer connexin protein can be validated and/or characterized using FETCH. In an aspect, a disclosed designer connexin protein can be validated and/or characterized using an animal model such as mice and/or C. elegans.

In an aspect, a disclosed designer connexin protein can restore the functionality and/or synchrony of a circuit (such as a neural circuit or a neuromuscular circuit).

In an aspect, a disclosed designer connexin protein can achieve context-precise circuit-editing characterized by a spatiotemporal specificity.

In an aspect, a disclosed designer connexin protein can synchronize electrical activity in a cellular circuit.

Disclosed herein is a designer connexin protein comprising the Cx34.7 connexin protein of Morone americana having a substitution at amino acid 214 and at amino acid 223.

In an aspect, a disclosed substitution at amino acid 214 can comprise a glutamic acid for a lysine (E214K). In an aspect, a disclosed substitution at amino acid 223 can comprise a glutamic acid for a lysine (E223K).

In an aspect, a disclosed Cx34.7 connexin protein can comprise the sequence set forth in SEQ ID NO:19 with a substitution at encoded amino acid 214 and at encoded amino acid 223.

In an aspect, a disclosed designer Cx34.7 connexin protein can comprise a nucleic acid sequence encoding a carboxy-terminal fluorescent label and/or fluorescent tag. In an aspect, a disclosed designer Cx34.7 connexin protein can comprise a nucleic acid sequence encoding an amino-terminal fluorescent label and/or fluorescent tag. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed designer Cx34.7 connexin protein can be validated and/or characterized using FETCH. In an aspect, a disclosed designer Cx34.7 connexin protein can be validated and/or characterized using an animal model such as mice and/or C. elegans

In an aspect, a disclosed designer Cx34.7 connexin protein can restore the functionality and/or synchrony of a circuit (such as a neural circuit or a neuromuscular circuit).

In an aspect, a disclosed designer Cx34.7 connexin protein can achieve context-precise circuit-editing characterized by a spatiotemporal specificity.

In an aspect, a disclosed designer Cx34.7 connexin protein can synchronize electrical activity in a cellular circuit.

Disclosed herein is a designer connexin protein comprising the Cx35 connexin protein of Morone americana having a substitution at amino acid 221.

In an aspect, a disclosed substitution at amino acid 221 can comprise a lysine for a glutamic acid (K221E). In an aspect, a disclosed Cx35 connexin protein can comprise the comprises the sequence set forth in SEQ ID NO:20 with a substitution at encoded amino acid 221.

In an aspect, a disclosed designer Cx35 connexin protein can comprise a nucleic acid sequence encoding a carboxy-terminal fluorescent label and/or fluorescent tag. In an aspect, a disclosed designer Cx35 connexin protein can comprise a nucleic acid sequence encoding an amino-terminal fluorescent label and/or fluorescent tag. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed designer Cx35 connexin protein can be validated and/or characterized using FETCH. In an aspect, a disclosed designer Cx35 connexin protein can be validated and/or characterized using an animal model such as mice and/or C. elegans.

In an aspect, a disclosed designer Cx35 connexin protein can restore the functionality and/or synchrony of a circuit (such as a neural circuit or a neuromuscular circuit).

In an aspect, a disclosed designer Cx35 connexin protein can achieve context-precise circuit-editing characterized by a spatiotemporal specificity.

In an aspect, a disclosed designer Cx35 connexin protein can synchronize electrical activity in a cellular circuit.

Disclosed herein is a fluorescently labeled designer connexin protein. Disclosed herein is a fluorescently labeled designer Cx34.7 protein. Disclosed herein is a fluorescently labeled designer Cx34.7_{M1} protein. Disclosed herein is a fluorescently labeled designer Cx35 protein.

Disclosed herein is a fluorescently labeled designer Cx35_{M1} protein.

In an aspect, a disclosed designer connexin protein can comprise a fluorescent label or a fluorescent tag. In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed fluorescently labeled designer connexin protein (such as a designer Cx34.7 connexin protein or a designer Cx35 connexin protein) can restore the functionality and/or synchrony of a circuit (such as a neural circuit or a neuromuscular circuit).

In an aspect, fluorescently labeled designer connexin protein (such as a designer Cx34.7 connexin protein or a designer Cx35 connexin protein) can achieve context-precise circuit-editing characterized by a spatiotemporal specificity.

In an aspect, a disclosed fluorescently labeled designer connexin protein (such as a designer Cx34.7 connexin protein or a designer Cx35 connexin protein) can synchronize electrical activity in a cellular circuit.

Disclosed herein is a pair of designer connexin proteins that can be used in a disclosed method. Disclosed herein is a pair of designer non-mammalian connexin proteins that can be used in a disclosed method. Disclosed herein is a pair of designer connexin proteins that can be used in a disclosed method of long-term integration of circuits.

In an aspect, a disclosed pair of designer connexin proteins can be any pair of connexin proteins that bind only to each other. In an aspect, a disclosed pair of designer connexin proteins can be any pair of connexin proteins in which each designer connexin protein does not bind homotypically to itself. In an aspect, each designer connexin in a disclosed pair fails to bind homotypically to itself. In an aspect, each designer connexin in a disclosed pair fails to bind to any wild-type or endogenous connexin in the mammalian CNS.

In an aspect, a disclosed pair of designer connexin proteins (such as, for example, Cx34.7_{M1} and Cx35_{M1}) can restore the functionality and/or synchrony of a circuit (such as a neural circuit or a neuromuscular circuit.

In an aspect, a disclosed pair of designer connexin proteins can achieve context-precise circuit-editing characterized by a spatiotemporal specificity.

In an aspect, a disclosed pair of designer connexin proteins can synchronize electrical activity in a cellular circuit.

### 3. Hemichannels

Disclosed herein are docked hemichannels comprising a first disclosed designer connexin and a second disclosed designer connexin.

Disclosed herein are docked hemichannels comprising a first designer connexin protein and a second designer connexin protein.

Disclosed herein are hemichannel combinations comprising a first labeled designer connexin and a second labeled designer connexin.

Disclosed herein are docked hemichannels comprising a Cx34.7 designer connexin protein and a Cx35 designer connexin.

Disclosed herein are docked hemichannels comprising a Cx34.7_{M1} designer connexin and a Cx35_{M1} designer connexin protein.

In an aspect, a disclosed hemichannel does not exhibit homotypic binding or homotypic docking. In an aspect, a disclosed hemichannel can be docking incompetent with a hemichannel endogenously expressed in the mammalian central nervous system.

In an aspect, a disclosed hemichannel comprising a first disclosed designer connexin protein can heterotypically bind only to a hemichannel comprising a second disclosed designer connexin protein.

In an aspect, a disclosed hemichannel comprising a disclosed designer Cx34.7 connexin protein can heterotypically bind only to a hemichannel comprising a disclosed designer Cx35 connexin protein.

In an aspect, a disclosed hemichannel comprising a disclosed Cx34.7M1 connexin protein can heterotypically bind only to a hemichannel comprising a disclosed Cx35_{M1} connexin protein.

In an aspect, a disclosed hemichannel can be validated and/or characterized using FETCH. In an aspect, a disclosed hemichannel can be validated and/or characterized using an animal model such as mice and/or C. elegans. In an aspect, a disclosed hemichannel can be validated and/or characterized using FETCH.

In an aspect, a disclosed hemichannel or disclosed pain of hemichannels can achieve context-precise circuit-editing characterized by a spatiotemporal specificity. In an aspect, a disclosed hemichannel or a disclosed pair of hemichannels can synchronize electrical activity in a cellular circuit.

Disclosed herein is a labeled connexosome comprising a first disclosed connexin and a second disclosed connexin. Disclosed herein is a labeled connexosome comprising a first disclosed designer connexin protein and a second disclosed designer connexin protein. Disclosed herein is a connexosome comprising a first disclosed designer connexin protein and a second disclosed designer connexin protein. Disclosed herein is a connexosome comprising a disclosed Cx34.7 designer connexin protein and a disclosed Cx35 designer connexin protein. Disclosed herein is a connexosome comprising a disclosed Cx34.7_{M1} connexin protein and a disclosed Cx35_{M1} connexin protein.

### 4. Gap Junctions

Disclosed herein is a synthetic gap junction comprising a first hemichannel heterotypically docked to a second hemichannel, wherein the first hemichannel comprises a first disclosed designer connexin protein, and wherein the second hemichannel comprises a second designer connexin protein.

Disclosed herein is a synthetic gap junction comprising a first disclosed hemichannel and a second disclosed hemichannel.

In an aspect of a disclosed synthetic gap junction, a first hemichannel can be expressed in a first cell type, and a second hemichannel can be expressed in a second cell type. In an aspect, a disclosed synthetic gap junction can exhibit rectification in a specific direction.

Disclosed herein is a synthetic gap junction comprising a hemichannel comprising a disclosed designer Cx34.7 connexin protein and a hemichannel comprising a disclosed designer Cx35 connexin protein.

Disclosed herein is a synthetic gap junction comprising a hemichannel comprising a disclosed designer Cx34.7_{M1} connexin protein and a hemichannel comprising a disclosed designer Cx35_{M1} connexin protein.

In an aspect, a disclosed synthetic gap junction can exhibit rectification in a Cx34.7_{M1} to Cx35_{M1} direction. In an aspect, a disclosed synthetic gap junction can restore the functionality and/or synchrony of a circuit (such as a neural circuit or a neuromuscular circuit).

In an aspect, a disclosed synthetic gap junction can achieve context-precise circuit-editing characterized by a spatiotemporal specificity. In an aspect, a disclosed synthetic gap junction can synchronize electrical activity in a cellular circuit.

In an aspect, a disclosed synthetic gap junction can be validated and/or characterized using FETCH. In an aspect, a disclosed synthetic gap junction can be validated and/or characterized using an animal model such as mice and/or C. elegans

### 5. Viral Vectors

Disclosed herein is a viral vector comprising a disclosed isolated nucleic acid molecule. Disclosed herein is a viral vector comprising an isolated nucleic acid molecule comprising a nucleic acid sequence encoding a designer connexin protein. Disclosed herein is a viral vector comprising an isolated nucleic acid molecule comprising a nucleic acid sequence encoding a designer Cx34.7 connexin protein. Disclosed herein is a viral vector comprising an isolated nucleic acid molecule comprising a nucleic acid sequence encoding a designer Cx35 connexin protein. Disclosed herein is a viral vector comprising an isolated nucleic acid molecule comprising a nucleic acid sequence encoding a designer Cx34.7_{M1} connexin protein. Disclosed herein is a viral vector comprising an isolated nucleic acid molecule comprising a nucleic acid sequence encoding a designer Cx35_{M1} connexin protein.

In an aspect of a disclosed viral vector, a disclosed encoded designer connexin protein can be derived from a non-mammalian species. In an aspect of a disclosed viral vector, a disclosed encoded designer connexin protein can be docking incompetent with a connexin endogenously expressed in the mammalian central nervous system. In an aspect, docking incompetent can mean not capable of docking with a connexin naturally expressed in a mammalian CNS. In an aspect of a disclosed viral vector, a disclosed encoded designer connexin protein can be capable of heterotypic binding or can heterotopically bind with another designer connexin protein. In an aspect of a disclosed viral vector, a disclosed encoded designer connexin protein can be not capable of homotypic binding or homotypic docking or cannot homotypically bind to the identical connexin protein.

In an aspect of a disclosed viral vector, a disclosed encoded designer connexin protein can comprise Cx34.7_{M1} or Cx35_{M1}. In an aspect, Cx34.7_{M1} can comprise the Cx34.7 of Morone americana having a substitution at amino acid 214 and at amino acid 223. In an aspect, a disclosed substitution at amino acid 214 can comprise a glutamic acid for a lysine (E214K). In an aspect, a disclosed substitution at amino acid 223 can comprise a glutamic acid for a lysine (E223K). In an aspect, Cx35_{M1} can comprise the Cx35 of Morone americana having a substitution at amino acid 221. In an aspect, a disclosed substitution at amino acid 221 can comprise a lysine for a glutamic acid (K221E). In an aspect, Cx34.7M1 can heterotypically bind only to Cx35M1. In an aspect, Cx35_{M1} can heterotypically bind only to Cx34.7M1.

In an aspect, wild-type Cx34.7 in Morone americana can comprise 306 amino acids. In an aspect, wild-type Cx34.7 in Morone americana can comprise the sequence set forth in GenBank Accession No. AAC31885.1 or a fragment thereof. In an aspect, the nucleotide sequence encoding wild-type Cx34.7 in Morone americana can comprise about 1871 bp. In an aspect, the nucleotide sequence encoding wild-type Cx34.7 in Morone americana can comprise the sequence set forth in GenBank Accession No. AF059184.1.

In an aspect, wild-type Cx35 in Morone americana can comprise 304 amino acids. In an aspect, wild-type Cx35 in Morone americana can comprise the sequence set forth in GenBank Accession No. AAC31884.1 or a fragment thereof. In an aspect, the nucleotide sequence encoding wild-type Cx35 in Morone americana can comprise about 1320 bp. In an aspect, the nucleotide sequence encoding wild-type Cx35 in Morone americana can comprise the sequence set forth in GenBank Accession No. AF059183.1.

In an aspect of a disclosed viral vector, a disclosed encoded designer connexin protein can comprise the sequence set forth in SEQ ID NO:19 with a substitution at encoded amino acid 214 and at encoded amino acid 223. In an aspect of a disclosed viral vector, a disclosed nucleic acid sequence encoding the designer Cx34.7 connexin protein can comprise the sequence set forth in SEQ ID NO:23 or SEQ ID NO:24 or a fragment thereof.

In an aspect of a disclosed viral vector, a disclosed encoded designer connexin protein can comprise the comprises the sequence set forth in SEQ ID NO:20 with a substitution at encoded amino acid 221. In an aspect of a disclosed viral vector, a disclosed nucleic acid sequence encoding the designer Cx35 connexin protein can comprise the sequence set forth in SEQ ID NO:25 or SEQ ID NO:26 or a fragment thereof.

In an aspect of a disclosed viral vector, a disclosed nucleic acid sequence encoding a designer connexin protein can be codon-optimized for expression in a mammalian cell or a human cell. In an aspect of a disclosed viral vector, a disclosed nucleic acid sequence encoding a designer connexin protein can be CpG-depleted and/or CpG-free.

In an aspect of a disclosed viral vector, a disclosed isolated nucleic acid molecule can comprise a nucleic acid sequence encoding a carboxy-terminal fluorescent label and/or fluorescent tag. In an aspect of a disclosed viral vector, a disclosed isolated nucleic acid molecule can comprise a nucleic acid sequence encoding an amino-terminal fluorescent label and/or fluorescent tag. In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art. In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect of a disclosed viral vector, a disclosed vector can restore the functionality and/or synchrony of a circuit (such as a neural circuit or a neuromuscular circuit).

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed viral vector can be an adenovirus vector, an adenovirus-associated (AAV) vector, or a lentivirus vector. In an aspect, a disclosed AAV vector can be a recombinant AAV (rAAV) vector.

In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1).

In an aspect, a disclosed nucleic acid sequence can have a coding sequence that is less than about 4.5 kilobases.

In an aspect, a disclosed viral vector can comprise a promoter operably linked to the isolated nucleic acid molecule. In an aspect of a disclosed viral vector, a disclosed promoter can comprise a tissue specific promoter. In an aspect, a disclosed tissue specific promoter can comprise a neuron-specific promoter, a muscle-specific promoter, a liver-specific promoter, a skeletal muscle-specific promoter, and heart-specific promoter. In an aspect, a disclosed tissue-specific promoter can comprise a brain cell specific promoter. In an aspect, a disclosed brain cell specific promoter can comprise a synapsin 1 (Syn1) promoter, a calmodulin/calcium dependent kinase II (CAMKII) promoter, a glial fibrillary acidic protein (GFAP) promoter, a Rgs5 promoter, a S100 beta promoter, a neuron-specific enolase (NSE) promoter, a Thy1 promoter, or any combination thereof. In an aspect, a disclosed ubiquitous promoter can be a CMV enhancer/chicken β-actin promoter (CB promoter). In an aspect, a disclosed promoter can be a promoter/enhancer.

In an aspect, a disclosed viral vector can comprise one or more regulatory elements. IN an aspect, a disclosed regulatory element can comprise a promoter, an enhancer, an internal ribosomal entry sites (IRES), and other expression control elements (e.g., transcription termination signals, such as polyadenylation signals and poly-U sequences). Regulatory elements can include those that direct constitutive expression of a nucleotide sequence in many types of host cells and those that direct expression of the nucleotide sequence only in certain host cells (e.g., tissue-specific regulatory sequences such as brain cells or neurons).

In an aspect, a therapeutically effective amount of disclosed vector can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, restoring the functionality and/or synchrony of a circuit (such as a neural circuit or a neuromuscular circuit).

In an aspect, a disclosed viral vector can achieve context-precise circuit-editing characterized by a spatiotemporal specificity. In an aspect, a disclosed viral vector can synchronize electrical activity in a cellular circuit.

In an aspect, a disclosed viral vector can be validated and/or characterized using FETCH. In an aspect, a disclosed viral vector can be validated and/or characterized using an animal model such as mice and/or C. elegans.

### 6. Cells

Disclosed herein is a cell comprising a disclosed isolated nucleic acid molecule or a disclosed plasmid. Disclosed herein are cells transfected by one or more disclosed nucleic acid molecules. Disclosed herein are cells transduced by one or more disclosed vectors.

In an aspect, disclosed transfected cells can comprise any mammalian central nervous system cells. CNS cells include but are not limited to neurons, glial cells, vascular cells, and combinations thereof. As known to the art, neurons include sensory neurons, motor neurons, interneurons, brain neurons, and combinations thereof. Neurons includes multipolar neurons, unipolar neurons, bipolar neurons, pseudo-unipolar neurons, and combinations thereof.

In an aspect, disclosed transfected cells can comprise HEK 293FT cells. HEK 293FT cells are known to the art. In an aspect, disclosed transfected cells can comprise any human cells (e.g., neurons, muscle fibers, etc.). In an aspect, disclosed transfected cells can comprise any cells that express connexins. In an aspect, disclosed transfected cells can comprise any cells that express connexins. In an aspect, disclosed transfected cells can comprise any cells that express a disclosed designer connexin. In an aspect, disclosed transfected cells can comprise any cells that express a disclosed designer connexin. Cells are known to the art. Cells are known to the art.

Disclosed herein are cells transfected by any plasmid set forth in Table 2. Disclosed herein are a pair of cells, each transfected by a plasmid set forth in Table 2.

Disclosed herein are cells transfected by an isolated nucleic acid molecule comprising the sequence set forth in any one of SEQ ID NO:23-SEQ ID NO:26 or a fragment thereof.

Disclosed herein are cells transfected by an isolated nucleic acid molecule comprising the sequence set forth in SEQ ID NO:21 or SEQ ID NO:22, which sequence comprises one or more mutations.

In an aspect, disclosed cells can comprise cells harvested and/or obtained from a subject. In an aspect, disclosed cells can comprise cells harvested and/or obtained from a subject suspected of having or diagnosed with a disease or disorder such as, for example, a disease or disorder characterized by gap junction dysfunction and/or gap junction pathophysiology.

Techniques to achieve transfection are known to the art and using transfected cells are known to the art.

### 7. Plasmids

Disclosed herein is a plasmid used in a disclosed method. Disclosed herein is a plasmid comprising one or more disclosed isolated nucleic acid molecules. Disclosed here are the plasmids of Table 2, which can be used in a disclosed method. Disclosed here are a pair of plasmids that can be used in a disclosed method, wherein the pair of plasmids are set forth in Table 2.

For example, in an aspect, a disclosed plasmid can comprise an isolated nucleic acid molecule comprising the sequence set forth in any one of SEQ ID NO:23-SEQ ID NO:26, or a fragment thereof. In an aspect, a disclosed plasmid can comprise an isolated nucleic acid molecule encoding the sequence set forth in SEQ ID NO:19 or SEQ ID NO:20, which sequence comprises one or more mutations. For example, in an aspect, a disclosed plasmid can comprise an isolated nucleic acid molecule comprising the sequence set forth in any one of SEQ ID NO:23-SEQ ID NO26, or a fragment thereof, and a nucleic acid sequence encoding a fluorescent label and/or tag. In an aspect, a disclosed plasmid can comprise an isolated nucleic acid molecule encoding the sequence set forth in any one of SEQ ID NO:27-SEQ ID NO:30, or a fragment thereof. In an aspect, a disclosed plasmid can comprise the sequence set forth in any one of SEQ ID NO:31-SEQ ID NO:40, or a fragment thereof.

In an aspect, a disclosed fluorescent label and/or fluorescent tag can comprise green fluorescent protein (EGFP), mEmerald, enhanced yellow fluorescent protein (EYFP), mApple, TdTomato, mCherry, miRFP670, any known fluorescent label or tag, or any combination thereof. In an aspect, a disclosed fluorescent label or disclosed fluorophore can comprise any fluorescent label or fluorophore that is amendable to analysis via flow cytometry. In an aspect, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

TABLE 2
Plasmids Comprising Nucleic Acid Sequences for Designer Cx Proteins

| SEQ ID # | Description of Sequence |
|---|---|
| 27 | Cx34.7-M1-mEmerald (codon optimized for human cells) |
| 28 | Cx34.7-M1-mEmerald (codon optimized for mouse cells) |
| 29 | Cx35-M1-mApple (codon optimized for human cells) |
| 30 | Cx35-M1-mApple (codon optimized for mouse cells) |
| 31 | Cx34.7-WT-mEmerald (pcDNA backbone with CMV promoter - codon |
| | optimized for human cells) |
| 32 | Cx35-WT-mApple (pcDNA backbone with CMV promoter - codon optimized |
| | for human cells) |
| 33 | Cx34.7-M1-mEmerald (pcDNA backbone with CMV promoter - codon |
| | optimized for human cells) |
| 34 | Cx35-M1-mApple (pcDNA backbone with CMV promoter - codon optimized |
| | for human cells) |
| 35 | Cx34.7-WT-mEmerald (pAAV backbone with CamKII promoter - codon |
| | optimized for mouse cells) |
| 36 | Cx35-WT-mApple (pAAV backbone with CamKII promoter - codon |
| | optimized for mouse cells) |
| 37 | Cx34.7-M1-mEmerald (pAAV backbone with CamKII promoter - codon |
| | optimized for mouse cells) |
| 38 | Cx35-M1-mApple (pAAV backbone with CamKII promoter - codon |
| | optimized for mouse cells) |
| 39 | Cx34.7-M1-mEmerald (pAAV-DIO backbone with Ef1a promoter - codon |
| | optimized for mouse cells) |
| 40 | Cx35-M1-mApple (pAAV-DIO backbone with Ef1a promoter - codon |
| | optimized for mouse cells) |

### 8. Kits

Disclosed herein is a kit comprising one or more disclosed isolated nucleic acid molecules, one or more disclosed proteins, one or more disclosed vectors, one or more disclosed cells, or any combination thereof. Disclosed herein is a kit comprising a disclosed designer connexin protein. Disclosed herein is a kit comprising a disclosed designer connexin protein comprising a label and/or a tag. Disclosed herein is a kit comprising one or more disclosed cells. Disclosed herein is a kit comprising one or more disclosed isolated nucleic acid molecules.

Disclosed herein is a kit comprising one or more disclosed cells transfected with a nucleic acid molecule comprising the sequence set forth in any one of SEQ ID NO:23-SEQ ID NO:26, or a fragment thereof.

Disclosed herein is a kit comprising one or more disclosed cells transfected with a nucleic acid molecule protein having the sequence set forth in any one of SEQ ID NO:27-SEQ ID NO:30, or a fragment thereof.

Disclosed herein is a kit comprising one or more disclosed cells transfected with a nucleic acid molecule comprising encoding a connexin protein comprising the sequence set forth in SEQ ID NO:19 or SEQ ID NO:20, which sequence comprises one or more mutations.

Disclosed herein is a kit comprising one or more disclosed cells transfected with a nucleic acid molecule encoding a designer Cx34.7 connexin protein comprising the sequence set forth in SEQ ID NO:19 with a substitution at encoded amino acid 214 and at encoded amino acid 223.

Disclosed herein is a kit comprising one or more disclosed cells transfected with a nucleic acid molecule encoding a designer Cx35 connexin protein comprising the comprises the sequence set forth in SEQ ID NO:20 with a substitution at encoded amino acid 221.

For example, in an aspect, a disclosed plasmid can comprise an isolated nucleic acid molecule comprising the sequence set forth in any one of SEQ ID NO:23-SEQ ID NO:26, or a fragment thereof. In an aspect, a disclosed plasmid can comprise an isolated nucleic acid molecule encoding the sequence set forth in SEQ ID NO:19 or SEQ ID NO:20, which sequence comprises one or more mutations. For example, in an aspect, a disclosed plasmid can comprise an isolated nucleic acid molecule comprising the sequence set forth in any one of SEQ ID NO:23-SEQ ID NO:26, or a fragment thereof, and a nucleic acid sequence encoding a fluorescent label and/or tag. In an aspect, a disclosed plasmid can comprise an isolated nucleic acid molecule having the sequence set forth in any one of SEQ ID NO:27-SEQ ID NO:30, or a fragment thereof. In an aspect, a disclosed plasmid can comprise the sequence set forth in any one of SEQ ID NO:27-SEQ ID NO:30, or a fragment thereof. In an aspect, a disclosed plasmid can comprise the sequence set forth in any one of SEQ ID NO:31-SEQ ID NO:40, or a fragment thereof.

Disclosed herein is a kit comprising one or more disclosed compositions and/or components and/or agents that can be used in any disclosed method.

Disclosed herein is a kit comprising one or more disclosed compositions and/or components and/or agents that can be used in validating and/or characterizing a disclosed composition (such as, for example, a disclosed isolated nucleic acid molecule, a disclosed designer connexin protein, a disclosed viral vector, or any combination thereof). In an aspect, validating and/or characterizing can comprise using an animal model such as mice and/or C. elegans.

In an aspect of a disclosed kit, a disclosed fluorescent label or a fluorescent tag. In an aspect, a disclosed fluorescent label or disclosed fluorophore can comprise enhanced green fluorescent protein (EGFP), mEmerald, enhanced yellow fluorescent protein (EYFP), mApple, TdTomato, mCherry, miRFP670, any known fluorescent label or tag, or any combination thereof. In an aspect of a disclosed kit, a disclosed fluorescent label or disclosed fluorophore can comprise any fluorescent label or fluorophore that is amendable to analysis via flow cytometry. In an aspect of a disclosed kit, a pair of disclosed fluorescent labels or disclosed fluorophores can comprise any pair of fluorescent labels or fluorophores that is amendable to analysis via flow cytometry and have excitation and emission spectra that can be isolated or separated from each other, thereby enabling the interrogation of the docking interactions. Fluorophores and fluorescent labels are known in the art.

In an aspect, a disclosed nucleic acid sequence encoding a designer Cx34.7 protein and a fluorescent tag can comprise the sequence set forth in SEQ ID NO:27 or SEQ ID NO:28. In an aspect, a disclosed nucleic acid sequence encoding a designer Cx35 protein and a fluorescent tag can comprise the sequence set forth in SEQ ID NO:29 or SEQ ID NO:30.

In an aspect, a disclosed kit can comprise at least two components constituting the kit. Together, the components constitute a functional unit for a given purpose (such as, for example, performing any aspect of a disclosed method including preparing the components used in a disclosed method). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed composition, a disclosed pharmaceutical formulation, a disclosed therapeutic agent, and a label or package insert with instructions for use. In an aspect, suitable containers include, for example, bottles, vials, syringes, blister pack, etc. The containers can be formed from a variety of materials such as glass or plastic. The container can hold a disclosed composition, a disclosed pharmaceutical formulation, a disclosed therapeutic agent, or a combination thereof, and can have a sterile access port (for example the container may be an intravenous solution bag or a vial having a stopper pierceable by a hypodermic injection needle). The label or package insert can indicate that a disclosed composition, a disclosed connexin, a disclosed nucleic acid molecule, a disclosed cell, or a combination thereof, can be used in a disclosed method. A kit can comprise additional components necessary for administration such as, for example, other buffers, diluents, filters, needles, and syringes. In an aspect, a disclosed kit can be used to evaluate gap junction formation, interrogate the docking interactions between connexin, perform high-throughput quantification of gap junction hemichannel docketing, or any combination thereof. In an aspect, a disclosed kit can be used in a method of restoring the functionality and/or synchrony of a circuit (such as a neural .circuit or a neuromuscular circuit).

In an aspect, a disclosed kit can comprise one or more disclosed plasmids, such as, for example, those plasmids identified in Table 2. In an aspect, a disclosed kit can comprise one or more disclosed primers, such as, for example, those plasmids identified in Table 2. In an aspect, a disclosed kit can comprise one or more populations of cells, each population transfected by a plasmid identified in Table 2.

### B. Methods

### 1. Methods of Precision Editing a Neural Circuit

Disclosed herein is a method of precision editing a neural circuit the method comprising generating a synthetic gap junction between a targeted first type of cells and targeted second type of cells in the central nervous system in a subject in need thereof, wherein the synthetic gap junction facilitates electrical activity between the first type of cells and the second type of cells.

In an aspect of a disclosed method of editing a neural circuit, generating a synthetic gap junction can comprise administering to the subject in need thereof a therapeutically effective amount of a first viral vector, wherein the first viral vector targets the first type of cells; and administering to the subject a therapeutically effective amount of a second viral vector, wherein the second viral vector targets the second type of cells, wherein the first type of cells is different than the second type of cells.

In an aspect, a disclosed first viral vector can comprise any disclosed vector. In an aspect, a disclosed second viral vector can comprise any disclosed vector.

In an aspect, a disclosed first viral vector can comprise a nucleic acid sequence encoding a Cx34.7 designer connexin protein. In an aspect, a disclosed first viral vector can comprise an isolated nucleic acid molecule comprising a nucleic acid sequence encoding a designer Cx34.7 connexin protein.

In an aspect, a disclosed second viral vector can comprise a nucleic acid sequence encoding a Cx35 designer connexin protein. In an aspect, a second viral vector can comprise an isolated nucleic acid molecule comprising a nucleic acid sequence encoding a designer Cx35 connexin protein.

In an aspect, a first viral vector can comprise an isolated nucleic acid molecule comprising a nucleic acid sequence encoding a designer Cx34.7_{M1} connexin protein. In an aspect, a second viral vector can comprise an isolated nucleic acid molecule comprising a nucleic acid sequence encoding a designer Cx35_{M1} connexin protein.

In an aspect, a disclosed method can comprise administering the first viral vector concurrently with the second viral vector. In an aspect, a disclosed method can comprise administering the first viral vector non-concurrently with the second viral vector. In an aspect, a disclosed method can comprise administering the first viral vector consecutively with the second viral vector. In an aspect, a disclosed method can comprise administering the first viral vector concurrently with the second viral vector.

In an aspect, following expression of the designer connexin protein transgene, the first type of cells can comprise a hemichannel comprising Cx34.7_{E214K/E223K}. In an aspect, following expression of the designer connexin protein transgene, the second type of cells can comprise a hemichannel comprising Cx35_{K221E}.

In an aspect, the electrical activity can rectify between the targeted first type of cells comprising CX34.7_{E214K/E223K} hemichannels and the targeted second type of cells comprising Cx35_{K221E} hemichannel. In an aspect, a disclosed synthetic gap junction exhibits rectification in the Cx34.7 to Cx35 direction.

In an aspect, a disclosed targeted first type of cells can comprise pre-synaptic neurons and wherein the targeted second type of cells can comprise post-synaptic neurons.

In an aspect of a disclosed method, the disclosed targeted first type of cells can remain subject to the control of its own inputs.

In an aspect of a disclosed method, the disclosed subject in need thereof can have or can have been diagnosed with a neurological disease and/or disorder. In an aspect of a disclosed method, the disclosed subject in need thereof can have or can have been diagnosed with a psychiatric disease and/or disorder. In an aspect of a disclosed method, the disclosed subject in need thereof can have or can have been diagnosed with a neuromuscular disease and/or disorder.

In an aspect of a disclosed method, the disclosed subject's behavior can be modulated. In an aspect of a disclosed method, the disclosed subject's physiology can be modulated. In an aspect of a disclosed method, the disclosed subject's cellular synchrony can be modulated.

In an aspect of a disclosed method, administering a disclosed viral vector can be administered via intravenous administration, intra-CSF administration, intracerebroventricular (ICV) administration, intraventricular administration, intra-cisterna magna (ICM) administration, intraparenchymal administration, intrathecal (lumbar, cisternal, or both) administration, or any combination thereof.

In an aspect of a disclosed method, administering a disclosed viral vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed viral vector can be administered via multiple routes of administration.

In an aspect, a therapeutically effective amount of disclosed vector (such as a first disclosed viral vector and/or a second disclosed viral vector) can comprise a range of about 1×10¹⁰ vg/kg to about 2×10¹⁴vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, restoring the functionality and/or synchrony of a circuit (such as a neural circuit or a neuromuscular circuit).

In an aspect, a disclosed method can achieve context-precise circuit-editing characterized by a spatiotemporal specificity. In an aspect, a disclosed method can synchronize electrical activity in a cellular circuit.

In an aspect, a disclosed method can comprise validating and/or characterizing the binding profile of a disclosed second designer connexin protein prior to administration to the subject in need thereof. In an aspect, validating and/or charactering the binding profile of a disclosed second designer connexin protein can comprise performing a FETCH analysis (discussed supra). In an aspect, validating and/or characterizing can comprise using an animal model such as mice and/or C. elegans.

In an aspect, a disclosed method can comprise validating and/or characterizing the binding profile and/or interaction of a pair of disclosed designer connexin proteins prior to administration to the subject in need thereof. In an aspect, validating and/or characterizing the binding profile and/or interaction of a pair of disclosed designer connexin proteins can comprise performing a FETCH analysis (discussed supra). In an aspect, validating and/or characterizing can comprise using an animal model such as mice and/or C. elegans.

In an aspect, a disclosed method of editing a neural circuit can further comprise monitoring the subject for adverse effects. In an aspect, wherein in the absence of adverse effects, the method can further comprise continuing to treat the subject and/or continuing to monitor the subject. In an aspect, wherein in the presence of adverse effects, the method can further comprise modifying one or more steps of the method. In an aspect, modifying can comprise modifying the treating step, modifying the administering step, or both.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

### 2. Methods of Precision Editing a Cellular Circuit

Disclosed herein is a method of precision editing a cellular circuit the method comprising generating a synthetic gap junction between a targeted first type of cells and targeted second type of cells in the cellular circuit in a subject in need thereof, wherein the synthetic gap junction facilitates electrical activity between the first type of cells and the second type of cells.

In an aspect of a disclosed method of editing a cellular circuit, generating a synthetic gap junction can comprise administering to the subject in need thereof a therapeutically effective amount of a first viral vector, wherein the first viral vector targets the first type of cells; and administering to the subject a therapeutically effective amount of a second viral vector, wherein the second viral vector targets the second type of cells, wherein the first type of cells is different than the second type of cells.

In an aspect, a disclosed first viral vector can comprise a nucleic acid sequence encoding a first designer connexin protein. In an aspect, a disclosed second viral vector can comprise a nucleic acid sequence encoding a second designer connexin protein.

In an aspect of a disclosed method of precision editing a cellular circuit, following expression of the designer connexin protein transgene, the first type of cells can comprise a hemichannel comprising a first designer connexin.

In an aspect of a disclosed method of precision editing a cellular circuit, following expression of the designer connexin protein transgene, the second type of cells can comprise a hemichannel comprising a second designer connexin.

In an aspect, the electrical activity can rectify between the targeted first type of cells comprising hemichannels comprising the first designer connexin and the targeted second type of cells comprising hemichannels comprising the second designer connexin.

n an aspect of a disclosed method of precision editing a cellular circuit, a disclosed targeted first type of cells can comprise pre-synaptic neurons and wherein the targeted second type of cells can comprise post-synaptic neurons. In an aspect of a disclosed method of precision editing a cellular circuit, the disclosed cellular circuit can comprise a neuromuscular junction. In an aspect of a disclosed method of precision editing a cellular circuit, the disclosed targeted first type of cells can comprise motor neurons and the disclosed targeted second type of cells can comprise muscle fibers.

In an aspect of a disclosed method of precision editing a cellular circuit, the disclosed targeted first type of cells can remain subject to the control of its own inputs.

In an aspect of a disclosed method, the disclosed subject in need thereof can have or can have been diagnosed with a neurological disease and/or disorder. In an aspect of a disclosed method, the disclosed subject in need thereof can have or can have been diagnosed with a neuromuscular disease and/or disorder. In an aspect of a disclosed method, the disclosed subject in need thereof can have or can have been diagnosed with a psychiatric disease and/or disorder. In an aspect of a disclosed method, the disclosed subject in need thereof can have or can have been diagnosed with gap junction dysfunction and/or gap junction pathophysiology.

In an aspect of a disclosed method, the disclosed subject's behavior can be modulated. In an aspect of a disclosed method, the disclosed subject's physiology can be modulated.

In an aspect of a disclosed method, administering a disclosed viral vector can be administered via intravenous administration, intra-CSF administration, intracerebroventricular (ICV) administration, intraventricular administration, intra-cisterna magna (ICM) administration, intraparenchymal administration, intrathecal (lumbar, cisternal, or both) administration, or any combination thereof.

In an aspect of a disclosed method, administering a disclosed viral vector can comprise a single dose, or in multiple doses (such as 2, 3, 4, 5, 6, 7, 8, 9 or 10 doses) as needed for the desired therapeutic results. In an aspect, multiple doses can be administered via the same route or via differing routes of administration. In an aspect, a disclosed viral vector can be administered via multiple routes of administration.

In an aspect, a therapeutically effective amount of disclosed vector (such as a first disclosed viral vector and/or a second disclosed viral vector) can comprise a range of about 1×10¹⁰ vg/kg to about 2×10¹⁴vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results (such as for example, restoring the functionality and/or synchrony of a circuit (such as a neural circuit or a neuromuscular circuit).

In an aspect, a disclosed method can achieve context-precise circuit-editing characterized by a spatiotemporal specificity. In an aspect, a disclosed method can synchronize electrical activity in a cellular circuit.

In an aspect, a disclosed method of precision editing a cellular circuit can comprise engineering the designer connexin protein.

In an aspect, engineering the designer connexins can comprise creating a library of mutants based on residues that confer and/or alter docking specificity.

In an aspect, engineering the designer connexins can comprise screening the mutants to identify those mutants having disrupted homotypic docking, wherein screening the mutants comprises subjecting the mutants to a FETCH analysis (discussed supra).

In an aspect, engineering the designer connexins can comprise identifying a pair of designer connexin protein mutants that demonstrate heterotypic docking, wherein identifying a pair of designer connexin protein mutants that demonstrates heterotypic docking comprises subjecting the pair to a FETCH analysis (discussed supra).

In an aspect, engineering the designer connexins can comprise probing the mutants identified in the FETCH analysis using a computational model of connexin protein docking.

In an aspect, a disclosed method can comprise validating and/or characterizing the binding profile of a disclosed first designer connexin protein prior to administration to the subject in need thereof. In an aspect, validating and/or characterizing the binding profile of a disclosed first designer connexin protein can comprise performing a FETCH analysis (discussed supra). In an aspect, validating and/or characterizing can comprise using an animal model such as mice and/or C. elegans.

In an aspect, a disclosed method can comprise validating and/or characterizing the binding profile of a disclosed second designer connexin protein prior to administration to the subject in need thereof. In an aspect, validating and/or characterizing the binding profile of a disclosed second designer connexin protein can comprise performing a FETCH analysis (discussed supra). In an aspect, validating and/or characterizing can comprise using an animal model such as mice and/or C. elegans.

In an aspect, a disclosed method can comprise validating and/or characterizing the binding profile and/or interaction of a pair of disclosed designer connexin proteins prior to administration to the subject in need thereof. In an aspect, validating the binding profile and/or interaction of a pair of disclosed designer connexin proteins can comprise performing a FETCH analysis (discussed supra). In an aspect, validating and/or characterizing can comprise using an animal model such as mice and/or C. elegans.

In an aspect, a disclosed method can further comprise administering to the subject a therapeutically effective amount of a therapeutic agent. Therapeutic agents are known.

In an aspect, a disclosed method can further comprise administering to the subject a therapeutically effective amount of one or more immune modulators. In an aspect, the one or more immune modulators comprise methotrexate, rituximab, intravenous gamma globulin, Tacrolimus, SVP-Rapamycin, bortezomib, or a combination thereof.

### VII. EXAMPLES

As described below, a novel class of electrical synapses have been created by selectively engineering two connexin proteins found in Morone americana (white perch fish): connexin34.7 (Cx34.7) and connexin35 (Cx35). By iteratively exploiting protein mutagenesis, using a novel in vitro assay of connexin docking, and employing computational modeling of connexin hemichannel interactions, a pattern of structural motifs that broadly determine connexin hemichannel docking have been identified. This knowledge was used to design Cx34.7 and Cx35 hemichannels that dock with each other, but not with themselves nor with other major connexins expressed in the human central nervous system. These hemichannels were validated in vivo using C. elegans and mice, demonstrating that the engineered connexins facilitated communication across neural circuits composed of pairs of genetically distinct cell types and modified behavior accordingly. As described below in the Specific Example, a translational approach called “Long-term integration of Circuits using connexins” or LinCx has been developed for context-precise circuit-editing with unprecedented spatiotemporal specificity in mammals.

The Examples that follow are illustrative of specific aspects of the invention, and various uses thereof. They set forth for explanatory purposes only and are not to be taken as limiting the invention.

### A. Materials and Methods Employed in Specific Examples

### 1. Design of Cx34.7 and Cx35 Mutant Library

A semi-rational design approach was used to design the mutant library. Sequence alignments between the Morone americana connexins and the connexins for which the most structure-function data existed (Cx26, Cx32, Cx36, Cx40, and Cx43) were performed in ClustalW. Sites identified by previous studies as conferring and/or altering specificity for docking were used as well as those identified by homology modeling from the structures of Cx26 (Koval M, et al. (2014) FEBS Lett. 588(8):1193-1204). Specifically, on the primary focus was on the extracellular loops and four residues at the interface in loop two, KEVE/KDVE (M. americana Cx34.7/Cx35) and one residue of E1. The homologous residues in other connexins had been demonstrated to be highly tolerant to mutation and critical for docking specificity (Jassim A, et al. (2016) J Mol Cell Cardiol. 90:11-20). Mutations were modeled in Swiss PDB Viewer using homology models of Cx34.7 and Cx35 from a Cx26 and Cx32 interface structure so as not to create mutations with obvious steric hindrance. A wide range of substitutions was made for these five residues of interest, including both those intended to introduce compatible electrostatic interactions as well as less likely candidates. Mutations were also created targeting other residues nearby and/or adjacent to these five for which there was some evidence in the literature that they contributed to docking specificity. However, the semi-rational approach was such that not as many variants were tried for these more distal site mutations and the mutations that were made in those sites were more conservative with regard to the steric and electrostatic properties of the change.

A listing of nucleotide and amino acid sequences is set forth in Table 3.

TABLE 3
Relevant Nucleotide and Amino Acid Sequences.

| SEQ ID # | Description of Sequence | Species |
|---|---|---|
| 1 | Cx36 Extracellular Loop #2 | Homo sapiens |
| 2 | Cx36 Extracellular Loop #2 | Mus musculus |
| 3 | Cx36 Extracellular Loop #2 | Macaca mulatta |
| 4 | Cx36 Extracellular Loop #2 | Callithrix jacchus |
| 5 | Cx36 Extracellular Loop #2 | Taeniopygia guttat |
| 6 | Cx36 Extracellular Loop #2 | Danio rerio (Cx35) |
| 7 | Cx43 Extracellular Loop #2 | Homo sapiens |
| 8 | Cx43 Extracellular Loop #2 | Mus musculus |
| 9 | Cx43 Extracellular Loop #2 | Macaca mulatta |
| 10 | Cx43 Extracellular Loop #2 | Callithrix jacchus |
| 11 | Cx43 Extracellular Loop #2 | Taeniopygia guttata |
| 12 | Cx43 Extracellular Loop #2 | Danio rerio |
| 13 | Cx45 Extracellular Loop #2 | Homo sapiens |
| 14 | Cx45 Extracellular Loop #2 | Mus musculus |
| 15 | Cx45 Extracellular Loop #2 | Macaca mulatta |
| 16 | Cx45 Extracellular Loop #2 | Callithrix jacchus |
| 17 | Cx45 Extracellular Loop #2 | Taeniopygia guttata |
| 18 | Cx45 Extracellular Loop #2 | Danio rerio |
| 19 | Wild-type Connexin34.7 | Morone americana |
| 20 | Wild-type Connexin35 | Morone americana |
| 21 | Wild-type Connexin34.7 (AF059184) | Morone americana |
| 22 | Wild-type Connexin35 (AF059183) | Morone americana |
| 23 | Cx34.7_{M1} (codon optimized for human cells) | Morone americana |
| | | (engineered) |
| 24 | Cx34.7_{M1} (codon optimized for mouse cells) | Morone americana |
| | | (engineered) |
| 25 | Cx35_{M1} (codon optimized for human cells) | Morone americana |
| | | (engineered) |
| 26 | Cx35_{M1} (codon optimized for mouse cells) | Morone americana |
| | | (engineered) |
| 27 | Cx34.7-M1-mEmerald (codon optimized for human | Artificial/Synthetic |
| | cells) | |
| 28 | Cx34.7-M1-mEmerald (codon optimized for mouse | Artificial/Synthetic |
| | cells) | |
| 29 | Cx35-M1-mApple (codon optimized for human cells) | Artificial/Synthetic |
| 30 | Cx35-M1-mApple (codon optimized for mouse cells) | Artificial/Synthetic |
| 31 | Cx34.7-WT-mEmerald (pcDNA backbone with | Artificial/Synthetic |
| | CMV promoter - codon optimized for human cells) | |
| 32 | Cx35-WT-mApple (pcDNA backbone with CMV | Artificial/Synthetic |
| | promoter - codon optimized for human cells) | |
| 33 | Cx34.7-M1-mEmerald (pcDNA backbone with CMV | Artificial/Synthetic |
| | promoter - codon optimized for human cells) | |
| 34 | Cx35-M1-mApple (pcDNA backbone with CMV | Artificial/Synthetic |
| | promoter - codon optimized for human cells) | |
| 35 | Cx34.7-WT-mEmerald (pAAV backbone with | Artificial/Synthetic |
| | CamKII promoter - codon optimized for mouse cells) | |
| 36 | Cx35-WT-mApple (pAAV backbone with CamKII | Artificial/Synthetic |
| | promoter - codon optimized for mouse cells) | |
| 37 | Cx34.7-M1-mEmerald (pAAV backbone with | Artificial/Synthetic |
| | CamKII promoter - codon optimized for mouse cells) | |
| 38 | Cx35-M1-mApple (pAAV backbone with CamKII | Artificial/Synthetic |
| | promoter - codon optimized for mouse cells) | |
| 39 | Cx34.7-M1-mEmerald (pAAV-DIO backbone with | Artificial/Synthetic |
| | Efla promoter - codon optimized for mouse cells) | |
| 40 | Cx35-M1-mApple (pAAV-DIO backbone with Efla | Artificial/Synthetic |
| | promoter - codon optimized for mouse cells) | |

### 2. Construct Cloning and Preparation

The initially acquired Morone Americana Cx34.7 and Cx35 cDNA constructs failed to efficiently express in in HEK 293FT cells. Thus, Connexin gene information was procured from the National Center for Biotechnology Information (NCBI, ncbi.nlm.nih.gov) and the Ensembl genome browser (ensembl.org). The human codon-optimized genes were ordered from Integrated DNA Technology as gBlocks Gene Fragments (IDT, idtdna.com). To generate constructs for transient transfection of HEK 293 FT cells, genes were subcloned into Emerald-N1 (addgene:53976) and piRFP670-N1 (addgene: 45457) vectors using In-Fusion cloning (takarabio.com), resulting in connexin fluorescent fusion proteins, specifically with the fluorescent proteins being adjoined to the connexin carboxy-terminus. Mutant constructs were generated by employing overlapping primers within standard Phusion polymerase PCR reactions to facilitate site-directed mutagenesis.

The Gateway recombination (Invitrogen) system was used to generate all Connexin 36, Cx34.7, Cx35, wild type and mutant protein C. elegans expression plasmids. For PCR-based cloning and subcloning of components into the Gateway system, either Phusion or Q5 High-Fidelity DNA-polymerase (NEB) was used for amplification. All components were sequenced within the respective Gateway entry vector prior to combining components into expression plasmids via a four-component Gateway system (Merritt C, et al. (2010) WormBook. 8:1-21). The different connexins versions were introduced into pDONR221a using a similar PCR-based strategy from plasmid sources (Chelur D S, et al. (2007) Proc Natl Acad Sci USA. 104(7):2283-2288; Chen T W, et al. (2013) Nature. 499(7458):295-300; Rabinowitch I, et al. (2014) Nat Commun. 5:4442). Cell-specific promoters were introduced using the pENTR 50-TOPO vector (Invitrogen) after amplification from genomic DNA or precursor plasmids. Transgenic lines were created by microinjection into the distal gonad syncytium (Mello C, et al. (1995) Methods Cell Biol. 48:451-482) and selected based on expression of one or more co-injection markers: Punc-122::GFP, Pelt-7::mCherry::NLS.

### 3. Cell Culture

HEK 293FT cells were purchased from Thermo Fisher Scientific (cat #R70007) and were maintained according to manufacturer instructions. Briefly, cultures were grown in 10 cm tissue culture treated dishes in high-glucose DMEM (Sigma Aldrich, D5796) supplemented with 6 mM L-glutamine, 0.1 mM MEM non-essential amino acids and 1 mM MEM sodium pyruvate in a 5% CO₂, 37° C. incubator. Cells were passaged via trypsinization every 2-3 days or until 60-80% confluency was reached.

### 4. FETCH Data Analysis

Complete FETCH methodology is outlined in Ransey et. al. 2021 (Ransey E, et al. (2021) BioRxiv). Briefly, replica multi-well plates with HEK 293FT cells were transfected with either of the two connexin proteins being evaluated for docking. Following a transfection incubation period, experimental counterparts were combined, replated, and further incubated for about 20 hr to about 24 hrs, allowing cells to make contacts and potentially generate connexosomes. Co-plated samples were trypsinized, fixed in suspension and analyzed via flow cytometry. Flow cytometry data was processed with two selection gates prior to their fluorescence evaluation. First, putative HEK cells were identified by evaluating sample forward vs. side scatter area. Next, single cells were identified as the putative cells that maintained a linear correlation of forward scatter height to forward scatter area. Finally, the dual fluorescence (mEmerald vs. RFP670) profiles of each sample was generated, and the FETCH score was defined as the proportion of dual-labeled fluorescent cells that develop in a co-plated sample.

For screening analysis, five FETCH replicates were obtained for each condition (mutation). These scores were benchmarked against scores for Cx36 and Cx45 (FETCH=1.2±0.1%, n=54 replicates). To quantitatively determine whether a connexin pair docked, FETCH scores were determined for the dual fluorescence of cells under conditions where docking was not anticipated. These conditions included pairs of connexins previously established to not show docking: (i) Cx36 and Cx45 (FETCH=0.7±0.0%, n=59 replicates), (ii) homotypic Cx23 (FETCH=0.9±0.4%, n=6 previously collected replicates (Ransey E, et al. (2021) BioRxiv), and (iii) Cx36 and Cx43 (FETCH=1.2±0.2%, n=10 previously collected replicates (Ransey E, et al. (2021) BioRxiv), and under conditions for which cells were transfected with cytoplasmic fluorophores rather than tagged connexins (FETCH=4.4±0.6%, n=17 previously collected replicates (Ransey E, et al. (2021) BioRxiv).

These 92 FETCH scores were used as the ‘known-negative’ distribution. FETCH scores from each experimental condition were then compared against the established negative score distribution using a one-tailed t-test, with a Bonferroni correction for the total number of experimental conditions tested. These FETCH replicates were independent of the replicates utilized for this screening analysis. Stats were reported as mean±s.e.m, and only uncorrected P values are reported through the text.

### 5. Confocal Imaging Analysis of Gap Junction Partners

For imaging of putative gap junction partners, different populations of HEK 293FT cells were transfected with counterpart connexin proteins, incubated, and combined as described for FETCH analysis. Combined samples of HEK 293FT cells were co-plated onto 10 μg/mL Fibronectin coated 35 mm, glass-bottom Mattek dishes (cat #P35GC-1.5-14-C). Cells were imaged at −20 hrs post co-plating. Images were acquired on a Leica SP5 laser point scanning inverted confocal microscope using Argon/2, HeNe 594 nm and HeNe633 nm lasers, conventional fluorescence filters and a 63X, HCX PL APO W Corr CS, NA: 1.2, Water, DIC, WD: 0.22 mm, objective. Images were taken with 1024×1024 pixels resolution at 200 Hz frame rate.

For assessing Cx34.7M1::Cx35_{M1} expression in vivo in C. elegans, strain DCR8669 olaEx5214 [Pgcy-8::CX34.7(E214K, E223K)::GFP; Pttx-3::CX35(K221E)::mCherry; Punc-122::GFP] was imaged. L4 animals were mounted in 2% agarose in M9 buffer pads and anaesthetized with 10 mM levamisol (Sigma). Confocal images were acquired with dual Hamamatsu ORCA-FUSIONBT SCMOS cameras on a Nikon Ti2-E Inverted Microscope using a confocal spinning disk CSU-W1 System, 488 nm and 561 nm laser lines and a CFI SR HP PLAN APO LAMBDA S 100×C SIL objective. Images were captured using the NIS-ELEMENTS software, with 2048 pixels×2048 pixels, 16-bit depth, 300 nm step size, 200 ms of exposure time and enough sections to cover the whole worm depth.

### 6. Protein Modeling Pipeline

The protein modeling pipeline was based on previously published methodology (Lee LD, (2018) PDXScholar) and integrated five components: (1) homology model generation, (2) embedding of proteins in a lipid bilayer and aqueous solution, (3) protein mutagenesis, (4) system minimization, equilibration, and molecular dynamics simulation production run, and (5) residue-wise energy calculation.

### 7. Homology Modeling

Five homology modeling software suites were initially tested: Robetta, SwissModel, Molecular Operating Environment (MOE; Chemical Computing Group ULC, Montreal, QC, Canada, H3A 2R7, 2021), I-Tasser, Phyre2 (Song Y, et al. (2013) Structure. 21(10):1735-1742; Waterhouse A, et al. (2018) Nucleic Acids Res. 46(W1):W296-W303; Bertoni M, et al. (2017). Sci Rep. 7(1):10480); Roy A, et al. (2010) Nat Protoc. 5(4):725-738); Yang J, et al. (2015) Nat Methods. 12(1):7-80; Yang J, et al. (2015) Nucleic Acids Res. 43(W1):W174-181; Kelley L A, et al. (2015) Nat Protoc. 10(6):845-858; Kallberg M, et al. (2012) Nat Protoc. 7(8):1511-1522); and Yang Y, et al. (2011) Bioinformatics. 27(15):2076-2082). A quality assessment suite, MOLProbity (Davis I W, et al. (2007) Nucleic Acids Res. 35(Supp 2):W375-W383); Chen V B, et al. (2010) Acta Crystallogr D Biol Crystallogr. 66(Pt 1):12-21; Williams C J, et al. (2018) Protein Sci. 27(1):293-315) revealed that Robetta models outperformed the rest, based on a set of standard metrics (Ramachandran plot outliers, clashscore, poor rotamers, bad bonds/angles, etc.).

Since the aim was to model the extracellular loops responsible for connexin hemichannel docking, all the resolved connexin structures that possessed a high degree of extracellular loop homology to the connexins of interest were chosen as the inputs for Robetta. The top homolog hits were generally the same for the three Cxs of interest: (i) Connexin-26 Bound to Calcium (5er7.1) (Bennett B C, et al. (2016) Nat Commun. 7:8770); (ii) Human Connexin-26 (Calcium-free) (5era) (Bennett B C, et al. (2016) Nat Commun. 7:8770); (iii) Structure of connexin-46 intercellular gap junction channel at 3.4 angstrom resolution by cryoEM (6mhq) (Myers J B, et al. (2018) Nature. 564(7736):372-377); (iv) Structure of connexin-50 intercellular gap junction channel at 3.4 angstrom resolution by cryoEM (6mhy) (Myers J B, et al. (2018) Nature. 564(7736):372-377), and (v) Structure of the connexin-26 gap junction channel at 3.5 angstrom resolution (2zw3) (Maeda S, et al. (2009) Nature. 458(7238):597-602). Cx34.7 and Cx35 wild type sequences had the greatest homology degree with 6mhq, while Cx36 was most homologous to 5er7.1. Three wild type hemichannels were generated for Cx34.7, Cx35, and Cx36.

### 8. System Assembly

Next, hemichannels were assembled into homotypic and heterotypic gap junctions, were embedded in two double bilayers, were dissolved in water, and appropriate ion concentrations were added for the extracellular and two intracellular compartments. The primary software suite used for this modeling step was VMD (Humphrey W, et al. (1996) J Mol Graph. 14(1):33-38; Eargle J, et al. (2006). Bioinformatics. 22(4):504-506). CHARMM GUI was utilized to generate the naturalistic model of a region of a double bilayer (Jo S, et al. (2008) J Comput Chem. 29(11):1859-1865; Wu E L, et al. (2014) J Comput Chem. 35(27):1997-2004; Jo S, et al. (2009) Biophys J. 97(1):50-58; Jo S, et al. (2007) PLoS One. 2(9):e880); Lee J, et al. (2019) J Chem Theory Comput. 15(1):775-786). Membrane components were then selected in appropriate proportions to resemble experimentally-derived data from a neuronal axonal membrane.

Specifically, since Robetta was unable to model the full gap junction, hemichannels into full homotypic/heterotypic gap junctions in a semi-automated way. First, to make homotypic gap junctions, the two-homology models for a hemichannel were loaded. The homology models were then aligned using the center of mass of the extracellular loops. A slight rotation along the z axis was implemented for several pairs to optimize fit. To make heterotypic gap junctions, a homotypic gap junction was created for each hemichannel, the extracellular loops for the two homotypic gap junctions were aligned, and then an opposing hemichannel from each homotypic gap junction was removed (leaving the two different hemichannels aligned).

Next, using the constructed gap junction, two pre-made membrane bilayers with the center of mass assigned as each embedded hemichannel were aligned. Then, membrane molecules that overlapped with the hemichannel or the hemichannel pore were removed. Next, the system was dissolved in water, and water that overlapped with the lipid bilayer was removed. Extracellular water was then separated to a new file, where Na+, K+, Cl−, and Ca2+ ions were added to yield concentrations mirroring the extracellular environment of mammalian neurons (Alberts B, et al. (2015)).

Finally, Na+, K+, Cl−, and Ca2+ ions were added to the intracellular space to mirror the intracellular environment of mammalian neurons, and the files containing the embedded Cx hemichannels and extracellular water were merged. Notably, these stages were automated yielding a streamlined progression from a protein-only hemichannel model to a fully embedded gap junction model ready for subsequent simulation and/or mutagenesis.

### 9. Mutagenesis

A python command-line tool that utilizes VMD to generate mutation configuration files was developed for subsequent MD simulation. Here, the connexin hemichannels of interest and the position at which a specific mutation should be introduced were simply specified.

### 10. System Minimization, Equilibration, and MD Simulation Production Run

Next atomic energies were minimized, the system was equilibrated, and the stable system was run in a production simulation run. Specifically, MD simulation was performed using NAMD (Phillips J C, et al. (2005) J Comput Chem, 2005. 26(16):1781-1802) and was divided into five steps: (1) Melt lipid tails while keeping remaining atoms fixed (simulate for 0.5 ns), (2) Minimize the system, then allow the bilayers and solutions to take natural conformation while keeping gap junction fixed (split in two stages to accommodate reduction in volume of relaxing system; simulate 0.5 ns total), (3) Release the gap junction and equilibrate the whole system (simulate 0.5 ns), (4) Run minimized and equilibrated system in a production run (simulate 0.5 ns). Though MD simulation (step 4) was highly reliant on the input file provided by the System Assembly process, these steps rendered the simulation much more robust to modeling imperfections. For example, the membrane model developed though System Assembly was very rigid and has the potential to behave like a solid rather than like a liquid. Thus, melting the lipid tails encouraged the model to embody a liquid. Similarly, many atoms in the input file may have unnatural initial energies, such that if they are all released at once, they would start moving at high velocities and the simulation would fail. Therefore, bringing the system to a local energy minimum increases stability. Removing constraints on the water and lipids enables them to surround the gap junction in a naturalistic form. Finally, releasing the constraints on the gap junction enables it to take the most energetically stable conformation given the environment.

### 11. Energy Calculation

To predict the residues that play a prominent role in docking, all non-bonding interactions were quantified between the two connexin hemichannels at key residues on the extracellular loops. Output from the MD simulation was loaded into the VMD “NAMD Energy” plugin. Nonbonding energies were then calculated for all residues on each hemichannel that were within 12 angstroms of at least one residue on the other hemichannel. For each residue pair, energies were then averaged across the 250 simulation frames.

12. C. elegans Strains and Genetics

Nematodes were cultivated at 20° C. on nematode growth medium seeded with a lawn of Escherichia coli strain OP50 using standard methods (Brenner S, et al. (1974) Genetics. 77(1):71-94). One-day-old adult hermaphrodites were used for all experiments. The strains used in this study are listed in the Strain Table (Table 4). All thermotaxis behavioral assays, molecular biology, transgenic lines, and calcium imaging were performed following the methods of Hawk et al. 2018 (Hawk J D, et al. (2018) Neuron. 97(2):e4), with minor modifications as outlined in the following subsections.

### 13. Thermotaxis Behavioral Assay

Animals were reared at 20° C. for all experiments with shifts to the 15° C. training temperature 4 hours prior to testing. High-throughput behavioral analyses were performed as described (Hawk J D, et al. (2018) Neuron. 97(2):e4), (Luo L, et al. (2014) Proc Natl Acad Sci USA. 111(7):2776-2781). Briefly, synchronized 1-day-old adult populations were washed in M9 buffer (Stiernagle T, (2006) WormBook), then transferred by pipette to the 20° C. isotherm of the behavioral test plate (22 cm×22 cm plates with a 18° C. to 22° C. thermal gradient). Each behavioral test plate was split in half along the temperature gradient using a thin and clear plastic divider. This allowed for wild type controls to be assayed on one half of the arena and connexin-expressing animals on the other half. Migration was monitored for 60 min at 2 fps using a MightEx camera (BCE-B050-U). Trajectories were analyzed using an adaptation of the MagatAnalyzer software package as previously described (Hawk J D, et al. (2018) Neuron. 97(2):e4; Luo L, et al. (2014) Proc Natl Acad Sci USA. 111(7):2776-2781; Gershow M, et al. (2012) Nature Methods. 9(3):290-296).

14. Calcium Imaging in C. elegans

For imaging, worms were mounted on a thin pad of 5% agarose in M9 buffer between two standard microscope coverslips. Worms were immobilized with 7.5 mM levamisol (Sigma). All solutions were pre-equilibrated to the holding temperature prior to sample preparation. Prepared coverslip assemblies were placed at TH on the peltier surface of the thermoelectric control system. A thermal probe (SRTD-2, Omega) mounted onto the surface of the peltier was used for feedback control via a commercially available proportional-integral-derivative (PID) controller (FTC200, Accuthermo). Target temperatures were supplied to the PID controller by a custom computer interface written in LabView (National Instruments), which subsequently gated current flow from a 12V power supply to the peltier via an H-bridge amplifier (FTX700D, Accuthermo). Excess heat was removed from the peltier with a water-cooling system. Precise temperature control was initially confirmed with an independent T-type thermal probe (IT-24P, Physitemp) attached to a hand-held thermometer (HH66U, Omega) and routinely compared to incubator temperatures with an infrared temperature probe (Actron). After mounting worms and placing them on the thermal control stage, fluorescence time-lapse imaging was begun immediately prior to implementing the temperature protocol in LabView, and temperature readings were recorded continuously while imaging. Fluorescence time-lapse imaging (250 ms exposure) was performed using a Leica DM5500 microscope with a 10×/0.40 HC PL APO air objective and a Hamamatsu ORCA-Flash4.0 LT camera. Image acquisition was performed using MicroManager (Edelstein A D, et al. (2014) J Biol Methods. 1(2)). Segmentation into regions of interest and downstream data processing was performed using FIJI (Schindelin J, et al. (2012) Nature Methods. 9(7):676-682), and custom scripts written in MATLAB (MathWorks), including alignment of fluorescence intensity values to the temperature stimulus, calcium response detection, and initial figure production. Temperature readings were assigned to image frames in MATLAB based on CPU timestamps on images and temperature readings. Heatmaps were displayed using the MATLAB ‘imagesc’ function, which scales data to the full color range available. The same scaling was applied to all comparable data within a figure. Unless otherwise specified, the scaling was based upon the highest intensity dataset to minimize saturation. For analyses of AFD calcium signals, an ROI of one AFD soma per animal was measured. For analyses of AIY, signal intensity in the synaptic Zone 2 was quantified. AIY is a unipolar neuron with both inputs, including those from AFD, in zone 2 and outputs to postsynaptic partners. A reference region outside of AIY was also quantified.

Responses were scored as the initial rise of the AFD or AIY calcium signal as determined by a blind human observer. Consistent with previous reports (Clark D A, et al. (2006) J Neurosci. 26(28):7444-7451), clear and qualitatively similar responses to thermal stimuli in the synaptic Zone 2 region were observed. The genetic background for the AFD and AIY calcium imaging lines used in this study (control and experimental) contained olaIs23, a caPKC-1 GOF mutation. This was done to match prior work (Hawk J D, et al. (2018) Neuron. 97(2):e4) in which Connexin 36 was demonstrated to evoke AFD-locked responses in AIY compared to caPKC-1 animals without Connexin 36.

### 15. Vertebrate Animal Care and Use

Male B6.129P2-Pvalb^{tml(cre)Arbr}/J (PV-Cre mice) and female C57BL/6J mice (Stock No: 017320 and 000664, respectively) purchased from Jackson labs were bred to generate male (n=16) and female (n=16) PV-Cre heterozygous mice utilized for the prefrontal cortex PYR↔PV+ editing experiment. Male C57BL/6J mice (n=29) purchased from Jackson were utilized for non-edited controls. Mice were housed three-five/cage on a 12-hour light/dark cycle and maintained in a humidity- and temperature-controlled room with water and food available ad libitum. Neural recordings were conducted during the dark cycle (Zeitgeber time: 13-19), given prior evidence that electrical synapse conductance can be diminished in the retina via circadian regulation. Inbred BALB/cJ male mice (strain: 000651) purchased from the Jackson Labs were used for infralimbic cortex→medial thalamus circuit editing experiments. This strain and sex of mice was chosen to mirror a previous study in which optogenetically targeting the infralimbic cortex→medial thalamus circuit (Carlson D, et al. (2017) Biol Psychiatry. 82(12):904-913). Behavioral experiments were conducted during the dark cycle (Zeitgeber time: 9-11). All vertebrate animal studies were conducted with University approved protocols and were in accordance with the federal guidelines for the care and use of laboratory animals.

### 16. Mouse Viral Infection Surgeries

PV-Cre mice were anesthetized with isoflurane (1%), placed in a stereotaxic device, and injected with a 1:1 solution of AAV9-CaMKII-Cx34.7_{M1}-mEmerald (titer: 5.0×10¹² vg/mL) and AAV9-Ef1α-DIO-Cx35_{M1}-mApple (titer: 1.3×10¹³ vg/mL), based on stereotaxic coordinates measured from bregma at the skull to target prelimbic cortex bilaterally (PrL: 2.1 mm AP, 0.65 mm ML, −1.45 mm DV from the dura at a 21° angle for male mice, and PrL: 2.05 mm AP, 0.62 mm ML, −1.41 mm DV from the dura at a 21° angle for female mice). A total of 1 μL viral solution was delivered to each hemisphere over 10 minutes using a 5 μL Hamilton syringe. Control mice were injected with a 1:1 solution of AAV9-CaMKII-Cx34.7M1-mEmerald and AAV9-Ef1α-DIO-Cx34.7_{M1}-mApple (titer: 1.1×10¹³ vg/mL) or a 1:1 solution of AAV9-CaMKII-Cx35_{M1}-mEmerald (titer: 6.9×10¹² vg/mL) and AAV9-Ef1α-DIO-Cx35M1-mApple. These viruses were created in the Duke Viral Vector Core. Viral infections were performed in male and female mice at age 2.5 months to 5 months, and viral manipulations were balanced across cages and sex. See Table 3 for relevant sequences identifiers.

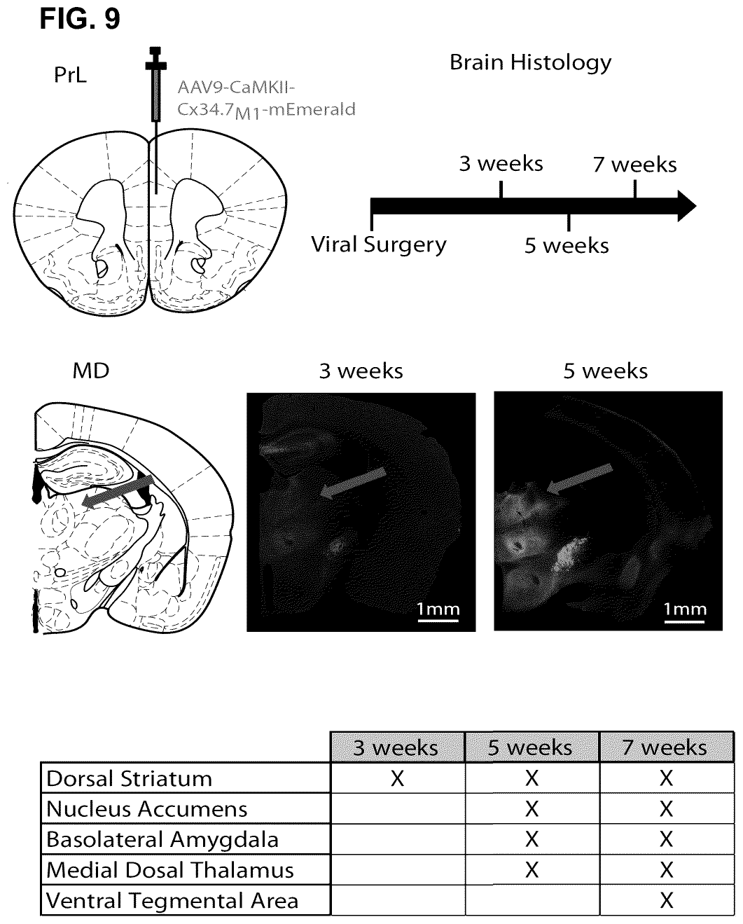

BALB/cj mice were anesthetized with isoflurane (1%), placed in a stereotaxic device, and injected with AAV9-CaMKII-Cx34.7_{M1}-mEmerald (titer: 5.0×1012 vg/mL) based on stereotaxic coordinates measured from bregma at the skull to target infralimbic cortex bilaterally (IL: 1.7 mm AP, 0.72 mm ML, −2.03 mm DV from the dura at an angle of 10°). A total of 0.5 μL viral solution was delivered to each hemisphere over 5 minutes using a 5 μL Hamilton syringe which was left in place for an additional 10 minutes prior to removal. Three weeks later (FIG. 9), mice were injected with AAV9-CaMKII-Cx35_{M1}-mApple (titer: 6.9×10¹² vg/mL) based on stereotaxic coordinates measured from bregma at the skull to target medial dorsal thalamus bilaterally (MD: −1.58 mm AP, 0.5 mm ML, −2.88 mm DV from the dura at an angle of 10°). Control mice were injected with AAV9-CaMKII-Cx34.7_{M1}-mEmerald in both IL and MD, or AAV9-CaMKII-Cx35_{M1} in both IL and MD, to express the synthetic hemichannels in non-docking homotypic configurations. Viral infections were performed in male mice at age 3 months. See Table 3 for relevant sequences identifiers.

### 17. Electrode Implantation Surgery

Mice were anesthetized with isoflurane (1.0%), placed in a stereotaxic device, and metal ground screws were secured to the cranium. For PV-Cre mice, a total of 8 tungsten microwires were implanted in prelimbic cortex (centered at 1.8 mm AP, ±0.25 mm ML, −1.75 mm DV from the dura for male mice; centered at 1.76 mm AP, ±0.25 mm ML, −1.71 mm DV from the dura for female mice). C57BL/6J control mice were implanted at 2 months. A total of 32 tungsten microwires were arranged as previously described multi-limbic circuit recording design (Mague S D, et al. (2020) BioRxiv). Briefly, bundles were implanted to target basolateral and central amygdala (AMY), medial dorsal thalamus (MD), nucleus accumbens core and shell (NAc), VTA, medial prefrontal cortex (mPFC), and VHip were centered based on stereotaxic coordinates measured from bregma (Amy: −1.4 mm AP, 2.9 mm ML, −3.85 mm DV from the dura; MD: −1.58 mm AP, 0.3 mm ML, −2.88 mm DV from the dura; VTA: −3.5 mm AP, ±0.25 mm ML, −4.25 mm DV from the dura; VHip: −3.3 mm AP, 3.0 mm ML, −3.75 mm DV from the dura; mPFC: 1.62 mm AP, ±0.25 mm ML, 2.25 mm DV from the dura; NAc: 1.3 mm AP, 2.25 mm ML, −4.1 mm DV from the dura, implanted at an angle of 22.1°). The cingulate cortex, prelimbic cortex, and infralimbic cortex were targeted using the mPFC bundle by building a 0.5 mm and 1.1 mm DV stagger into the electrode bundle microwires. Animals were implanted bilaterally in mPFC and VTA. All other bundles were implanted in the left hemisphere. The NAc bundle included a 0.6 mm DV stagger such that wires were distributed across NAc core and shell. The BLA and CeA were targeted by building a 0.5 mm ML stagger and 0.3 mm DV stagger into theAMY electrode bundle (Mague S D, et al. (2020) BioRxiv).

### 18. Neurophysiological Data Acquisition

Neural recordings experiments were performed in PV-Cre mice one week after implantation surgery and blind to viral group. Mice were habituated to the recording room for at least 60 minutes prior to testing. PV-Cre mice were connected to a headstage (Blackrock Microsystems, UT, USA) without anesthesia, and given a single saline injection (10 mL/kg mouse, intraperitoneally). Notably, these saline injections were performed to facilitate comparison of acquired neural data with future drug studies. Twenty-five minutes later, mice were placed in a 17.5 in×17.5 in×11.75 in (L×W×H) chamber for 60 minutes. Recordings were conducted under low illumination conditions (1−2 lux), and only data from the first 10 minutes of exposure to the open field were used for neurophysiological analysis. For C57BL/6J control mice, experiments were performed at least two weeks following implantation surgery. Mice were habituated to the recording room for at least 60 minutes prior to testing, and headstages were connected without anesthesia. Twenty-six male mice were placed in a 17.5 in ×17.5 in×11.75 in (L×W×H) chamber for 10 minutes, and three mice were recorded in a 19.5 in×12 in (D×H) circular chamber. Six of these mice were injected with (10 mL/kg mouse, intraperitoneally) 30 minutes prior to recordings, and all recordings were conducted under an illumination of 125 lux.

Neuronal activity was sampled at 30 kHz using the Cerebus acquisition system (Blackrock Microsystems Inc., UT). Local field potentials (LFPs) were bandpass filtered at 0.5 Hz-250 Hz and stored at 1000 Hz. All neurophysiological recordings were referenced to a ground wire connected to both ground screws, and an online noise cancellation algorithm was applied to reduce 60 Hz artifact.

### 19. Determination of LFP Cross Frequency Phase Coupling

Signals recorded from all viable implanted microwires were used for analysis. Local field potentials were filtered using 4th order Butterworth bandpass filters designed to isolate theta (4 Hz-10 Hz) prefrontal cortex oscillations and high frequency oscillations (80 Hz-200 Hz). The instantaneous amplitude and phase of the filtered LFPs were then determined using the Hilbert transform, and the Modulation index was calculated for each LFP channel using the MATLAB code provided by Canolty et al (Canolty R T, et al. (2006) Science. 313(5793):1626-1628). Briefly, a continuous variable z(t) is defined as a function of the instantaneous theta phase and instantaneous gamma amplitude such that z(t)=A_{G}(t)*e^{iϕ}_{TH}^{(t)}, where A_{G} is the instantaneous gamma oscillatory amplitude, e^{iϕ}_{TH} is a function of the instantaneous theta oscillatory phase. A time lag was then introduced between the instantaneous amplitude and theta phase values such that zₛᵤᵣᵣ is parameterized by both time and the offset between the two variables, zₛᵤᵣᵣ=A_{HG}(t+τ)*e^{iϕ}_{TH}^{(t)}. The modulus of the first moment of z(t), compared to the distribution of surrogate lengths, provided a measure of coupling strength. The normalized z-scored length, or Modulation index, was then defined as M_{NORM}=(M_{RAW}−μ)/σ, where M_{RAW} was the modulus of the first moment of z(t), μ was the mean of the surrogate lengths, and a was their standard deviation (Carlson D, et al. (2017) Biol Psychiatry. 82(12):904-913), (Dzirasa K, et al. (2010). J Neurosci. 30(48):16314-16323), (Canolty R T, et al. (2006) Science. 313(5793):1626-1628). The modulation index scores were averaged across all implanted channels for each mouse (˜7.3 channels for each PV-Cre mouse, and 2 channels implanted bilaterally for each C57BL/BJ control mouse).

### 20. Behavioral Testing

All behavioral testing was conducted under low illumination conditions (1-2 lux). Two weeks after the second viral surgery, mice were initially placed in a 17.5 in×17.5 in×11.75 in (L×W×H) chamber for five minutes of open field testing. Mice were suspended 1 cm from the tip of their tail for six minutes. Open field and TST neurophysiological data were acquired during a single testing session, and the behavior testing session was repeated the next day. Testing sessions were video recorded, and open field and tail suspension behavior was analyzed using EthoVision ABC. Behavioral experiments and subsequent video analyses were performed blind to group.

### B. Specific Examples

Many molecular events at synapses regulate the communication between cells, and studies have linked changes in synchrony across brain circuits to cognitive and emotional behaviors. For example, electrical oscillations in hippocampus and prefrontal cortex synchronize during spatial memory in rats (Jones M W, et al. (2005) PLoS Biol. 3(12):e402), and between amygdala and hippocampus during fear memory retrieval in mice (Seidenbecher T, et al. (2003) Science. 301(5634):846-850). Moreover, altered long range synchrony has been observed in preclinical rodent models of schizophrenia (Sigurdsson T, et al. (2010) Nature. 464(7289):763-767), depression (Hultman R, et al. (2018) Cell. 173(1):e14), and autism (Wang X, et al. (2016) Nat Commun. 7:11459) and manipulating synchrony between infralimbic cortex and thalamus has been shown to enhance resilience to acute stress (Carlson D, et al. (2017) Biol Psychiatry. 82(12):904-913). Though these studies highlight the therapeutic potential for modulating synchrony to drive long lasting changes in behavior, it remains a major challenge to manipulate the activity of precise brain circuits with sufficient precision to elicit synchrony. Both spatial (i.e., the interaction of specific cells in space) and temporal constraints of endogenous neural circuits (i.e., the ongoing integrated activity across multiple circuits) make this goal especially challenging. For example, classic pharmacological therapeutics targeting ion receptors or neuromodulators pathways impact many brain circuits, and emerging brain stimulation-based approaches directly impact the activity of many neuronal populations (i.e., the rate code) and the timing of activity between distinct cells that comprise neural circuits (i.e., the timing code as reflected by synchrony). As such, tools that selectively regulate synchrony within specific circuits in animals have been sparse.

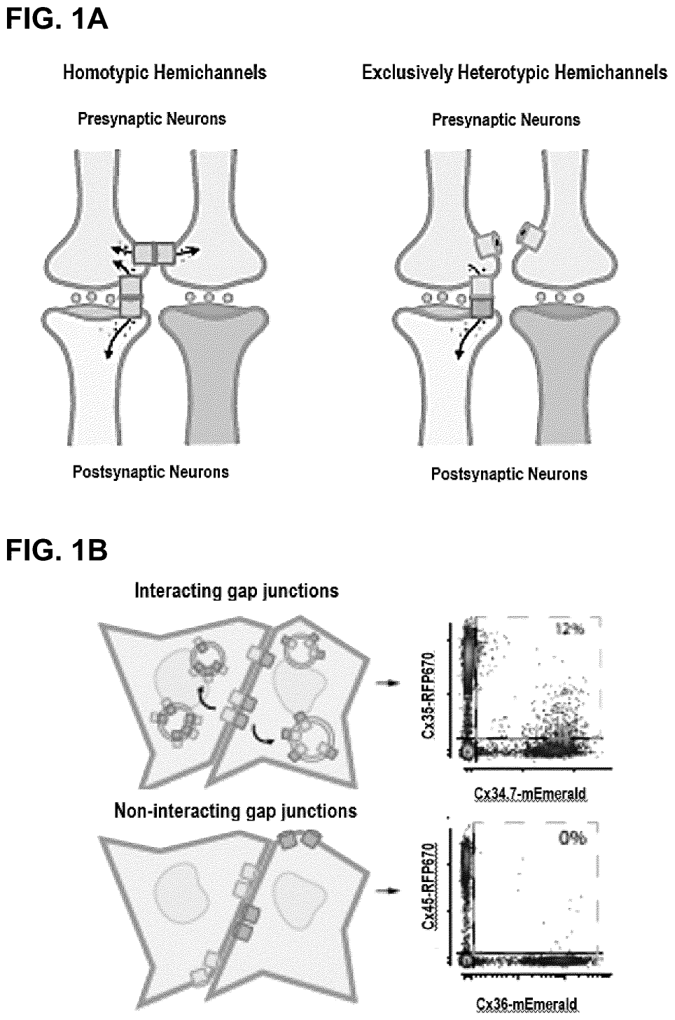

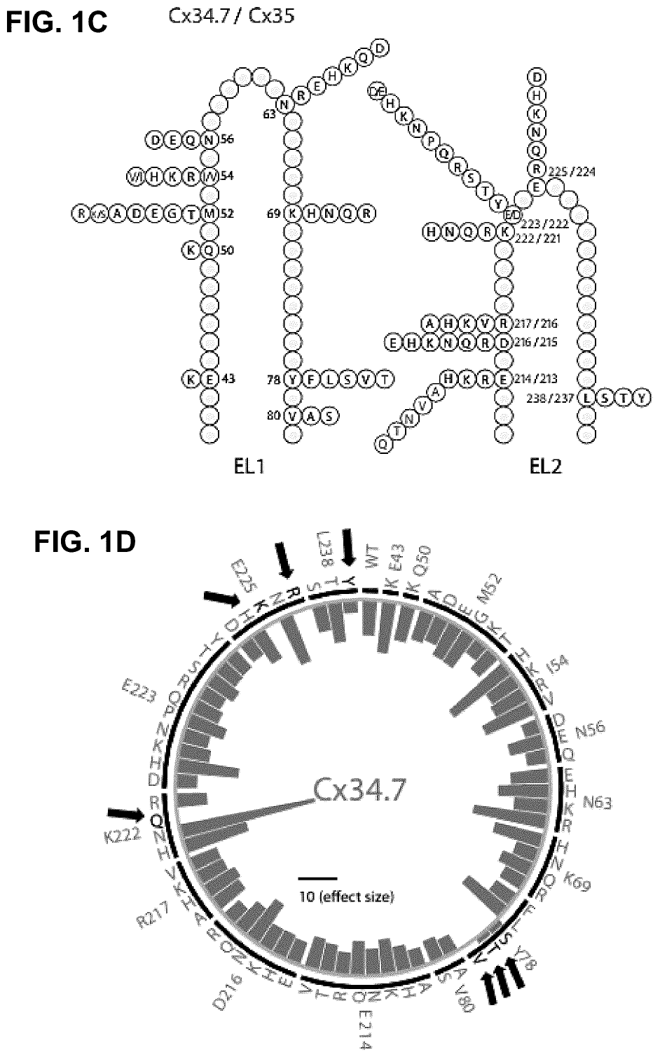

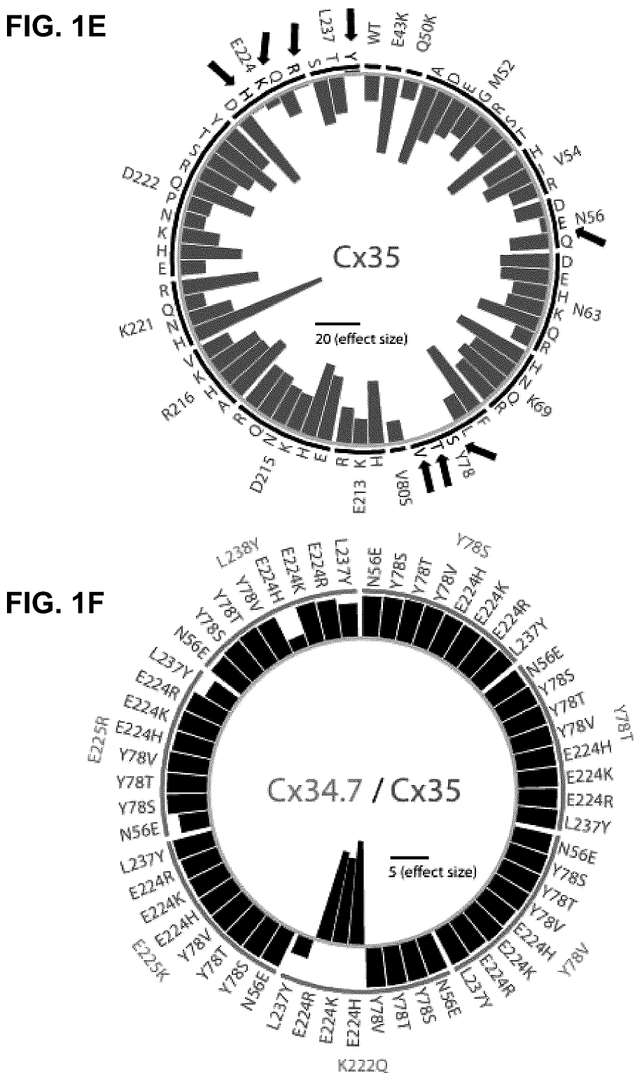

Gap junctions (electrical synapses) enable direct flow of ions and small molecules between two cells and play a prominent role in broadly synchronizing electrical activity in many organs such as the heart and the brain (Alcami P, et al. (2019) Nat Rev Neurosci. 20(5):253-271). To achieve such synchrony, gap junction consists of two docked segments called hemichannels, embedded in the membranes of two adjoining cells. Each hemichannel, in turn, is an oligomer consisting of six monomeric proteins called connexins (Cx), of which there are 21 isoforms in humans (Sohl G, et al. (2003) Cell Commun Adhes. 10(4-6):173-180; Sohl G, et al. (2004) Cardiovasc Res. 62(2):228-232). Many Cxs can form single-isoform hemichannels that dock with themselves to create homotypic gap junctions (FIG. 1A (left)). This common feature limits the potential utility of Cxs as tools to selectively regulate brain circuit activity in complex organisms. Expression of ectopic Cx proteins that form homotypic synapses would establish connections between individual neurons of the same cell type, though they may operate in distinct neural circuits (FIG. 1A). In such a scenario, information flow would flow orthogonally across otherwise independent circuits. On the other hand, several Cx hemichannels are capable of recognizing hemichannels composed of other Cx protein isoforms and can generate heterotypic, gap junctions (FIG. 1A (right)) (Laird D W, et al. (2006) Biochem J. 394(Pt 3):527-543; Koval M, et al. (2014) FEBS Lett. 588(8):1193-1204). Thus, it was reasoned that an exclusively heterotypic hemichannel pairing would provide substantial control over the direction of information flow between distinct cell types, and that mechanistic characterization of homotypic and heterotypic hemichannel docking may support the rational design of such a pair (FIG. 1A). It was also reasoned that knowledge of these mechanisms could be utilized to engineer connexin proteins that were docking incompetent to the major connexins endogenous to the mammalian CNS, a feature that would be essential for any tools developed for precise circuit editing in mammalian species.

Morone americana (white perch fish) express two homologs of mammalian neuronal connexin36 (Cx36)—Cx34.7 and Cx35—that create heterotypic electrical synapses between auditory afferents and Mauthner cells in the CNS (Rash J E, et al. (2013). Neuron. 79(5):957-969). Critically, the Cx34.7/Cx35 heterotypic gap junction exhibits rectification in the Cx34.7 to Cx35 direction (Rash J E, et al. (2013). Neuron. 79(5):957-969), with an intrinsic conductance that increases above the threshold (i.e., −20 mV) for action potential firing in the brains of mammals (O'Brien J, et al. (1998) J Neurosci. 18(19):7625-7637). Since this former feature of the Cx34.7/Cx35 heterotypic gap junction enables a preferred directional modulation that mirrors the general operation of chemical synapses, Cx34.7 and Cx35 were chose for this platform to engineer the synthetic electrical synapse. The residues responsible for Cx34.7 and Cx35 docking were systematically altered to identify two mutant hemichannels that exhibit heterotypic but not homotypic interactions, and do not dock with other connexin proteins endogenous to the mammalian CNS. This synthetic synapse was deployed in C. elegans to demonstrate its functionality and specificity as a neuronal circuit editing tool.

### Example 1

### In Vitro Analysis of Connexin Protein Docking

Though the precise interactions that guide hemichannel docking are incompletely characterized for the majority of connexins, structure-function and sequence analyses indicated that the second extracellular loop (EL2) plays the greatest role in hemichannel docking specificity (Gong X Q, et al. (2013) J Cell Sci. 126(Pt14):3113-3120). An in vitro approach for rapid evaluation of connexin hemichannel docking (Ransey E, et al. (2021) BioRxiv) was developed. Here, it was reasoned that this approach could be used to rapidly screen a library of mutations for the ability to disrupt homotypic docking for the two mutants. Subsequently, a subset of non-homotypic docking Cx34.7 and Cx35 mutants could be screened against each other to identify putative heterotypic docking pairs.

The rapid screening approach (dubbed Flow Enabled Tracking of Connexosomes in HEK cells or FETCH), enables rapid assessment of docking for many combinations of connexin proteins using flow cytometry. Specifically, FETCH utilizes the presence of connexosomes (a by-product of gap junction formation) as an indicator of docking compatibility. Connexosomes are double-bilayer, vesicular structures composed of fully-docked gap junctions that are internalized into each cell contributing to gap junctions, as part of normal turnover (FIG. 1B) (Jordan K, et al. (2001) J Cell Sci. 114(Pt4):763-773; Falk M M, et al. (2016) BMC Cell Biol. 17 Suppl 1:22). Thus, docking compatibility of different fluorescently-labeled Cxs can be characterized as a function of the fluorescence exchange between cells, mediated by connexosomes (Ransey E, et al. (2021) BioRxiv). In this FETCH assay, individual Cxs are expressed as either GFP-fluorescent fusion proteins or RFP670-fluorescent fusion proteins in HEK cells. Following an initial expression, HEK cells that are expressing intended Cx counterparts are then co-plated and incubated. This allows apposed Cxs to potentially interact, and fluorescence exchange is evaluated using flow cytometry. Docking is characterized as the proportion of dual-labeled fluorescent cells that develop in a co-plated sample.

To identify potential mutations that destabilize Cx34.7 and Cx35 homotypic hemichannel docking, seventy mutations were rationally introduced at sixteen positions on the extracellular loops (ELs) of Cx34.7 while sixty-seven mutations at sixteen positions were rationally introduced on both extracellular loops (ELs) of Cx35 (see methods for mutant library design). The homotypic docking interactions of these mutant proteins were then quantified using FETCH screening (FIG. 1C). Here, the goal was to identify residues that completely disrupted docking, and thus, the mutant scores were benchmarked against a heterotypic pairing of human Cx36 and Cx45 proteins that has previously been shown not to yield functional gap junctions (Li X, et al. (2008) J Neurosci. 28(39):9769-9789). Critically, for this screening, whether these mutations directly disrupted docking interactions or indirectly disrupted docking via interference with an upstream process such as folding or trafficking was not considered. Homotypic FETCH screening revealed that most mutants retained their docking character; however, several non-homotypic docking mutant proteins were identified. Non-homotypic Cx34.7 mutants included Y78S, Y78T, Y78V, E225K, E225R, L238Y and K222Q, and non-homotypic Cx35 mutants included N56E, Y78V, Y78S, Y78T, E224H, E224K, E224R, and L237Y (FIG. 1C-FIG. 1E)

Next, to identify mutant protein pairs that exhibit exclusively heterotypic docking, the Cx34.7 and Cx35 non-homotypic mutants were screened against each other using FETCH. Here, a successful docking interaction verified that upstream process such as folding or trafficking remained intact for both Cx mutants. Strikingly, three connexin mutant pairs whose FETCH scores were higher than the FETCH scores observed for wild type Cx34.7_{WT}/Cx35_{WT} gap junctions were discovered. These results provided clear evidence that it was indeed feasible to engineer a connexin hemichannel pairs that demonstrated heterotypic, but not homotypic, docking. Interacting mutant pairs were comprised of Cx34.7_{K222Q} with either Cx35_{E224H}, Cx35_{E224K}, or Cx35_{E224R} as counterparts (FIG. 1F).

Since the long-term objective was to develop a modulation approach that would be amenable for use in the mammalian nervous system, whether the four mutant isoforms docked with endogenous connexin isoforms—specifically Cx36 and connexin43 (Cx43), the major connexins in mammalian neurons and astrocytes, respectively (Condorelli D F, et al. (2000) Brain Res Brain Res Rev. 32(1):72-85; Rash J E, et al. (2001). J Neurosci. 21(6):1983-2000), was also probed. Using heterotypic FETCH analysis, none of the mutant proteins interacted with human Cx43 (FETCH=1.3±0.1%; T₉₆=0.29; p=0.61 for Cx34.7_{K222Q}/Cx43 compared to established non-docking pair replicates using a one tailed t-test, with a Bonferroni correction for 20 comparisons; FETCH=0.4±0.1% (Cx35_{E224H},/Cx43), 0.5±0.1% (Cx35_{E224K}/Cx43), and 0.5±0.1% (Cx35_{E224R}/Cx43); T₉₆=1.41 (Cx35_{E224H},/Cx43), 1.36 (Cx35_{E224K}/Cx43), and 1.28 (Cx35_{E224R}/Cx43); p=0.92 (Cx35_{E224H},/Cx43), 0.91 (Cx35_{E224K}/Cx43), and 0.90 (Cx35_{E224R}/Cx43); n=6 replicates for all experimental connexin pairs; FETCH=1.5±0.2%, n=92 for established non-docking pairs; see methods). However, Cx34.7_{K222Q} and Cx35_{E224H} interacted with human Cx36 (FETCH=22.8±1.9%; T₉₁=−24.4; p=3.4×10⁻⁴³ for Cx34.7_{K222Q}/Cx36; FETCH=5.9±1.1% (Cx35_{E224H}/Cx36), 0.8±0.1% (Cx35_{E224K}/Cx36), and 0.6±0.1% (Cx35_{E224R}/Cx36); T₉₆=−5.50 (Cx35_{E224H}/Cx36), 0.89 (Cx35_{E224K}/Cx36), and 1.24 (Cx35_{E224R}/Cx36); p=1.6×10⁻⁷ (Cx35_{E224H}/Cx36), p=0.81 (Cx35_{E224K}/Cx36), and p=0.89 (Cx35_{E224R}/Cx36).

### Example 2

### Rational Design of Exclusively Heterotypic and Isoform Specific Cx34.7 and Cx35 Pair Using in Silico Modeling

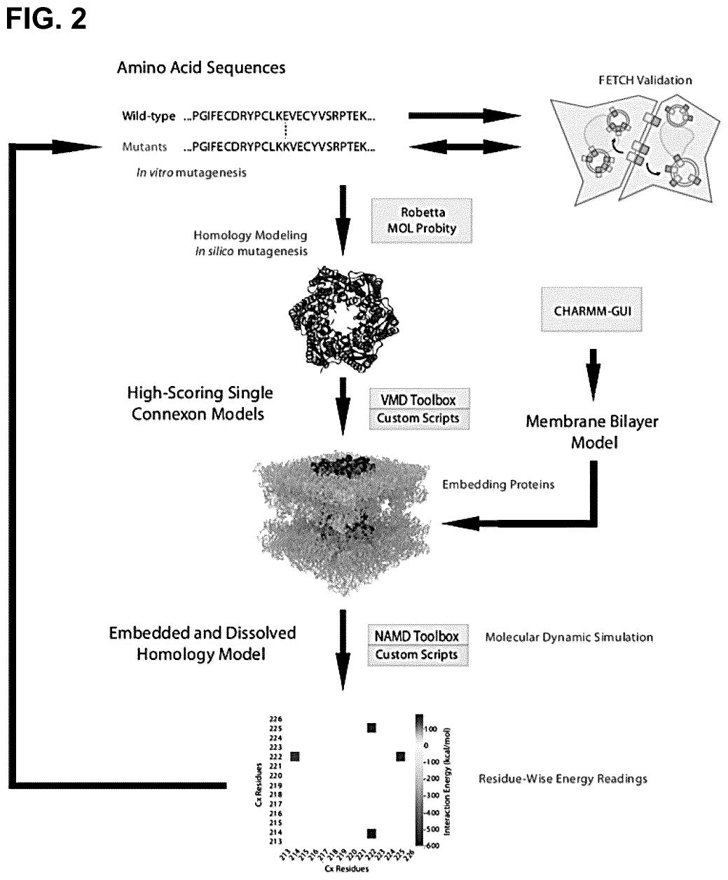

To address the unintended docking of Cx34.7_{K222Q} with Cx36 determined via FETCH, homology modeling was used to rationally design a heterotypically exclusive connexin pairs that does not interact with endogenous Cx43 and Cx36. Briefly, computational models of the Cx34.7 and Cx35 wild type and mutant, homotypic and heterotypic gap junctions that were tested in the initial FETCH screen were developed. The computational models were validated by comparing the key residues predicted to underly hemichannel docking against the initial docking characteristics of the mutants that were determined using FETCH. Next, the docking interactions between Cx34.7_{K222Q}, Cx35_{E224H}, Cx35_{E224K}, and Cx35_{E224R} mutant proteins and Cx36 were modeled. Finally, the insights of residue-wise interaction models were used to design Cx34.7 and Cx35 hemichannels that would exhibit exclusively heterotypic docking and would not dock with Cx36. The docking characteristics of this designer Cx34.7 and Cx35 proteins were then generated in vitro using FETCH (FIG. 2).

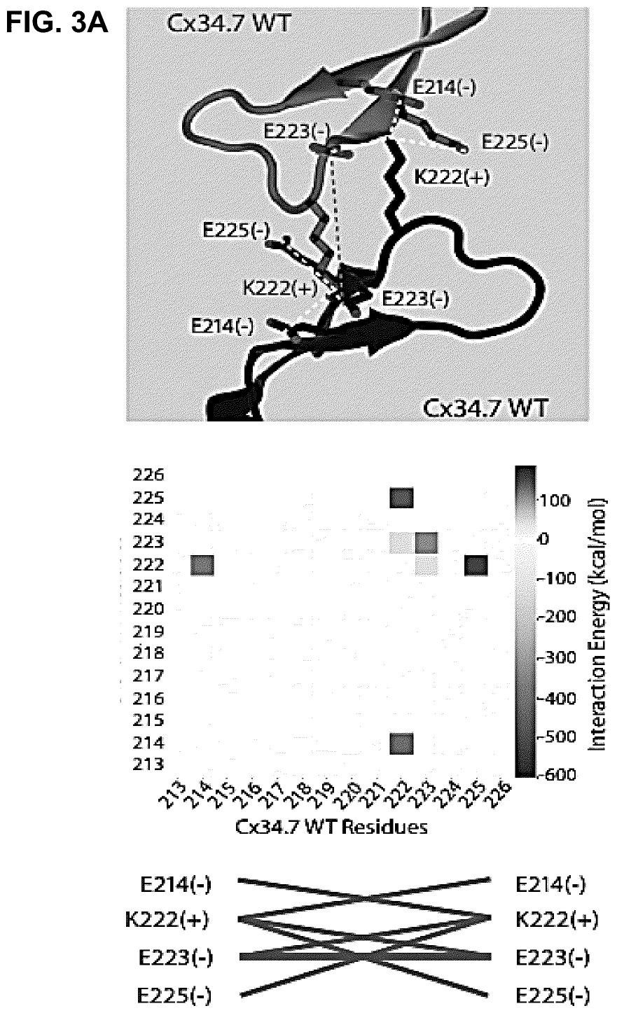

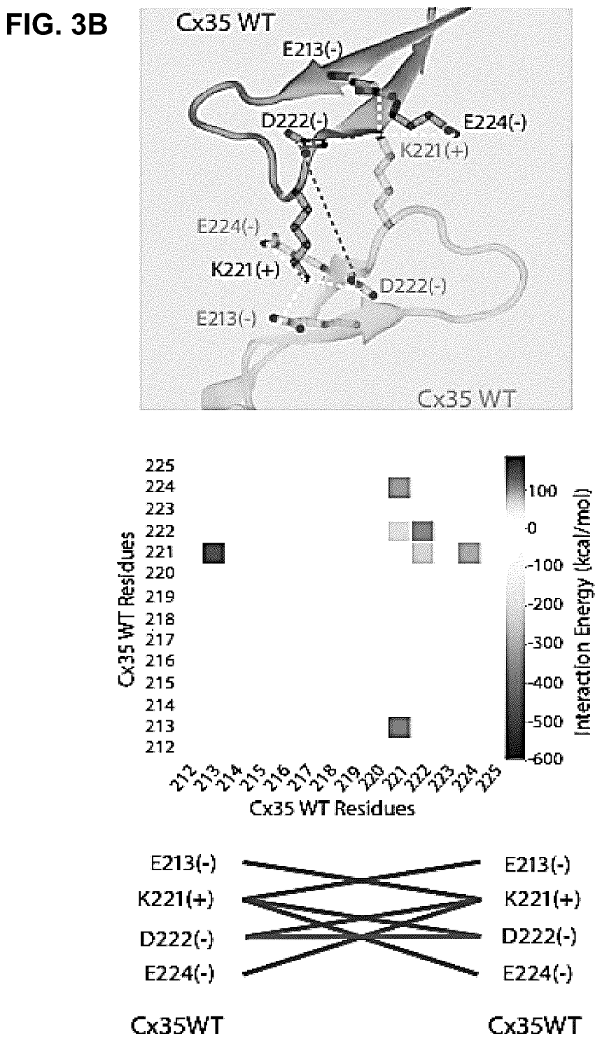

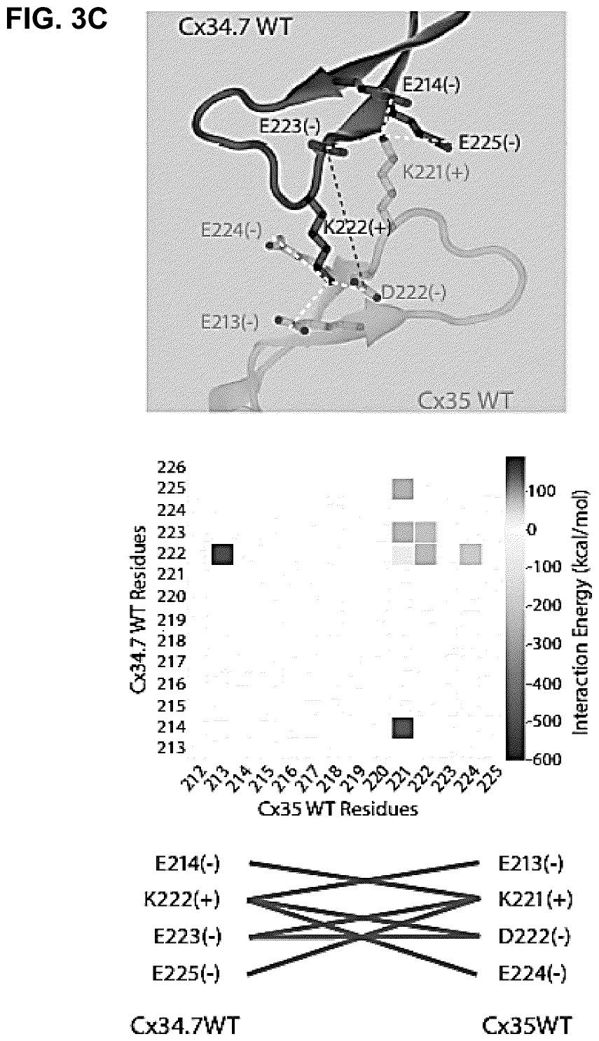

Specifically, to model docking interactions between Cx34.7 and Cx35 gap junctions, molecular dynamics simulations of modeled homotypic and heterotypic pairs of wild type and mutant proteins (Cx34.7 and Cx35) were run. This modeling revealed large negative interaction energies involving residues E214, K222, E223, and E225 in wild type Cx34.7 and residues E213, K221, D222, E224 in wild type Cx35 for both the homotypic and heterotypic docking simulations. These large negative interaction energies were indicative of salt bridges that stabilize both homotypic and heterotypic docking interactions, since results from the initial homotypic FETCH screens showed that mutations of Cx34.7-K222 and Cx35-E224 disrupted docking. Providing further support for this observation, residues in EL2 that had the lowest interaction energies in the wild type pairs showed much higher energy in mutants for which the charge was switched (e.g., Cx35_{E224R}, Cx35_{D222H}, Cx35_{D222R}, and Cx34.7_{E223K}), or for which a smaller residue of the same charge was introduced (e.g., Cx34.7_{K222H}). Integrating these results, a common interaction motif for both Cx34.7 and Cx35 consisting of three negative residues for Cx34.7 (E214/E223/E225) and for Cx35 (E213/D222/E224) was observed as was a positive residue for Cx34.7 (K222) and for Cx35 (K221) (FIG. 3A-FIG. 3C). Thus, the four targeted residues were determined to play a prominent role in docking interactions for wild type homotypic and heterotypic Cx34.7 and Cx35 gap junctions.

Next, Cx36 was introduced into the computational modeling pipeline to characterize its homotypic and heterotypic interaction principles. Upon simulating the homotypic Cx36 gap junction, the same pattern of interactions was observed for Cx34.7 and Cx35, with residues E230, K238, E239, and E241 serving as key interaction sites. Given that the predicted Cx36 interaction motif was identical to that of Cx34.7, it was unsurprising that Cx36 was additionally modeled to interact with Cx34.7 and Cx35 heterotypically. After verifying these heterotypic docking interactions via FETCH (FETCH=11.9±1.2%; T₉₆=−12.93; p=4.7×10⁻²³ for Cx34.7/Cx36; FETCH=18.0±2.0%; T₉₆=−18.69; p=4.7×10⁻³⁴ for Cx35/Cx36), the four Cx mutants identified in the initial FETCH analysis (Cx34.7_{K222Q}, Cx35_{E224H}, Cx35_{E224K}, Cx35_{E224R}) were introduced into the computational framework and modelled their modified heterotypic interactions with Cx36.

Mutating K222Q in Cx34.7 disrupted the large negative interaction energies that were observed in the homotypic wild type Cx34.7 model, consistent with the findings from FETCH analysis that this mutation impaired homotypic docking (FETCH=0.4±0.1%; T₉₆=1.43; p=0.92 homotypic Cx34.7_{K222Q}). On the other hand, the three negative residues in Cx34.7_{K222Q} continued to show large negative interaction energies with the positive residue of Cx36, explaining the preserved heterotypic docking between Cx34.7_{K222Q} and Cx36 observed via FETCH. Screened mutant Cx35 proteins (Cx35_{E224H}, Cx35_{E224K}, and Cx35_{E224R}) maintained the positive K221 residue that formed strong interactions with the negative residues of Cx36; however, the Cx35_{E224K}, and Cx35_{E224R} mutations induced strong repulsion with the positive K238 residue of Cx36, explaining why these two mutants failed to heterotypically dock with Cx36 in the FETCH analyses. Interestingly, introducing a smaller positive charge at the same position, Cx35_{E224H} restored the interaction with Cx36 in the computational model, and docking in the heterotypic FETCH analyses.

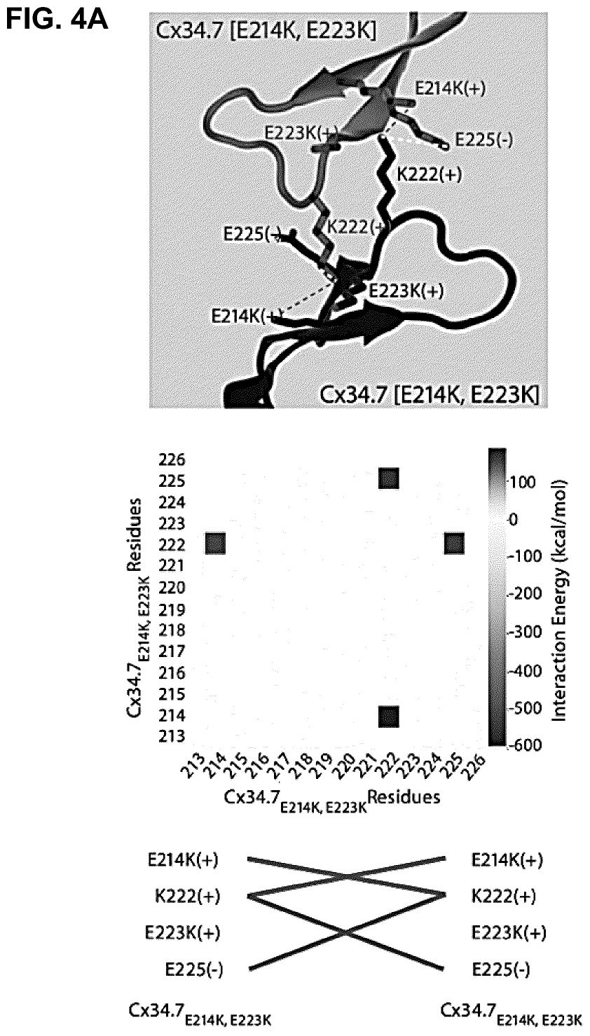

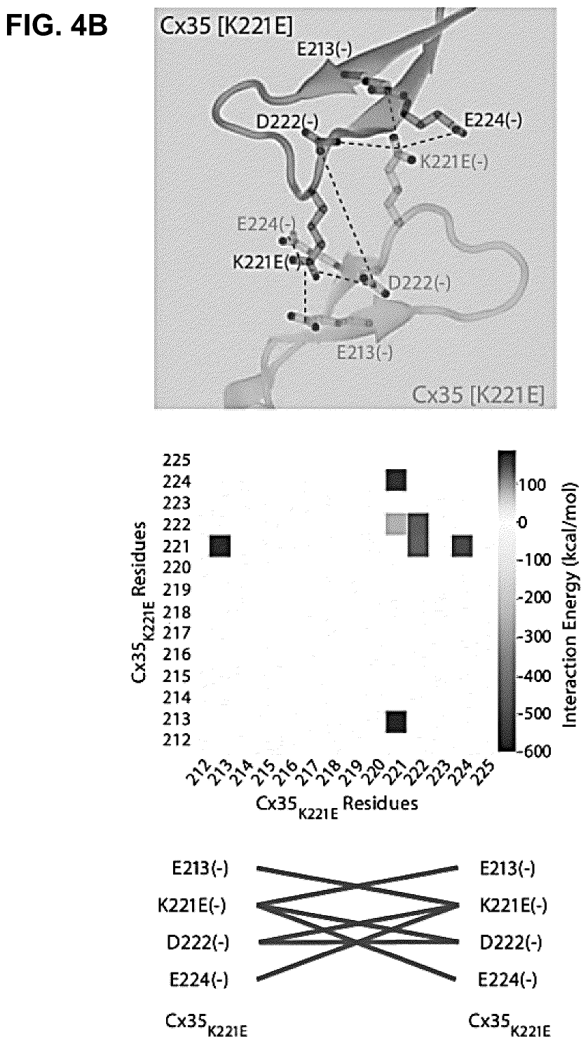

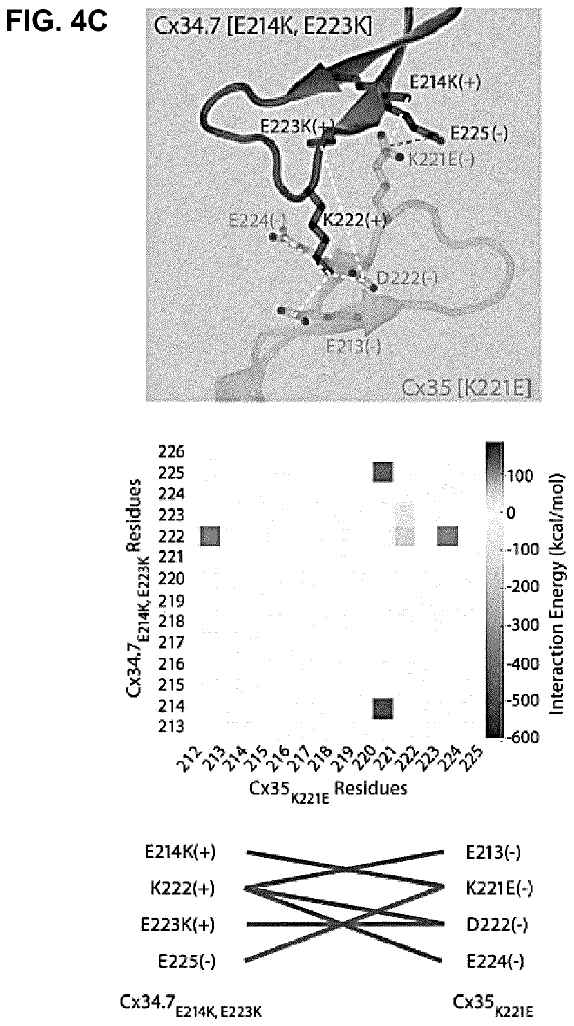

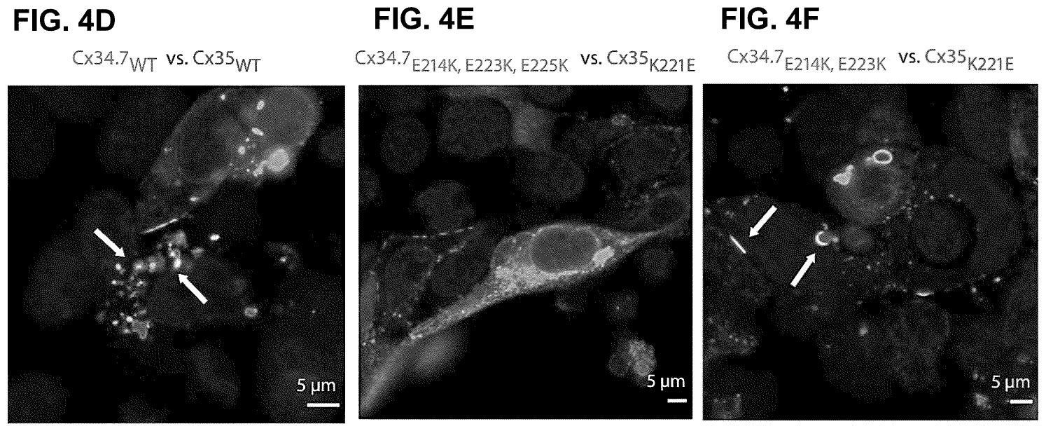

Having determined putative interaction principles underlying the docking of the three connexin homologs (Cx34.7, Cx35, and Cx36), rationally designing a Cx34.7/Cx35 pair that would exhibit isoform-specific, exclusively heterotypic docking was undertaken. The initial strategy was to mutate residues at the four positions of the identified docking motif such that one connexin isoform contained all negative charge interactors, and the counterpart connexin contributed all positive interactors. Cx35_{K221E} (which had all negative charges at motif residues) showed strong repulsions in the homotypic model, and it did not exhibit homotypic docking in FETCH analysis (FETCH=1.2±0.4%; T₉₆=0.35; p=0.64). Additionally, this mutant protein failed to dock with Cx36 and Cx43 (FETCH=1.5±0.1%, T₉₁=0.02, p=0.51 for Cx35_{K221E}/Cx36 FETCH=1.7±0.2, T₉₆=−0.32, p=0.37 for Cx35_{K221E}/Cx43). Similarly, Cx34.7_{E214K/E223K/E225K} (which had all positive charges at motif residues) showed strong repulsions in the homotypic computation model and it did not exhibit homotypic docking in FETCH analysis (FETCH=0.2±0.0%; T₉₆=1.76; p=0.96). However, when Cx35_{K221E} was paired with Cx34.7_{E214K, E223K, E225K} for heterotypic FETCH analysis, the two mutant proteins did not dock (FETCH=1.2±0.3; T₉₆=0.37; p=0.64), despite the model predicting strong interaction. Follow-up confocal imaging analysis of HEK 293FT cells expressing the constructs revealed that Cx34.7_{E214K, E223K, E225K} failed to properly localize to the cell membrane (compare FIG. 4D and FIG. 4E). This failed mutant was not tested against Cx36 or Cx43; but rather, an intermediate Cx34.7 mutant protein that exhibited strong positive charges at only three of the residues, Cx34.7_{E214K/E223K} was evaluated. This mutant showed strong attractive interactions with Cx35_{K221E} in the computational model, and it localized to the cell membrane where it docked with Cx35_{K221E} as confirmed by FETCH analysis and confocal microscopy (FETCH=35.7±4.1%; T₉₆=−28.11; p=2.0×10⁻⁴⁸; see FIG. 4F for confocal image). Critically, Cx34.7_{E214K/E223K} did not show homotypic docking in the FETCH analysis (FETCH=1.1±0.2%, T₉₆=0.46, p=0.68), nor did it dock with Cx36 or Cx43 for Cx34.7_{E214K/E223K}/Cx36 (FETCH=1.0±0.2, T₉₆=0.58, p=0.72) and Cx34.7_{E214K/E223K}/Cx43 (FETCH=0.9±0.1%, T₉₆=0.73, p=0.77). Going forward, this rationally designed, exclusively heterotypic and isoform-specific connexin pair (Cx34.7_{E214K/E223K} and Cx35_{K221E}) was referred to as Cx34.7_{M1}/Cx35_{M1} (designer Cxs version 1.0, from Morone americana).

### Example 3

### Synthetic Cx34.7_{M1}/Cx35_{M1} Electrical Synapses Regulated Behavior

Using these novel designer Cxs, whether Cx34.7_{M1}/Cx35_{M1} could establish functional gap junctions with electrical properties sufficient to synchronize the activity of distinct neurons that compose a circuit was examined. C. elegans was used for this analysis because the nematode nervous system is composed of well-characterized circuits of individual cells that regulate behavior. C. elegans do not have an innate temperature preference and can thrive in a broad range of temperatures (Hedgecock E M, et al. (1975) Proc Natl Acad Sci USA. 72(10):4061-4065). However, C. elegans trained at a temperature in the presence of food will migrate towards that temperature when they are subsequently placed on a temperature gradient without food (Hedgecock E M, et al. (1975) Proc Natl Acad Sci USA. 72(10):4061-4065). This learned behavioral preference is—in part—mediated by plasticity of the chemical synapse occurring between a thermosensory neuron (called AFD, presynaptic) and an interneuron (called AIY, postsynaptic) (Mori I, et al. (1995) Nature. 376(6538):344-348). Critically, plasticity in the thermosensory neuron AFD can be genetically manipulated to affect transmission to AIY, and to predictably code the otherwise learned behavioral preference (Hawk J D, et al. (2018) Neuron. 97(2):e4).

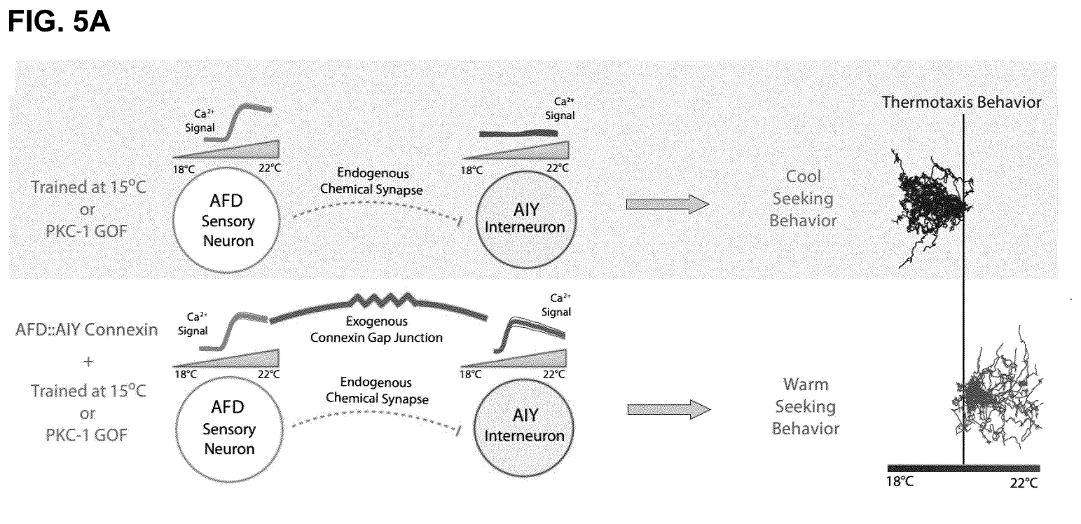

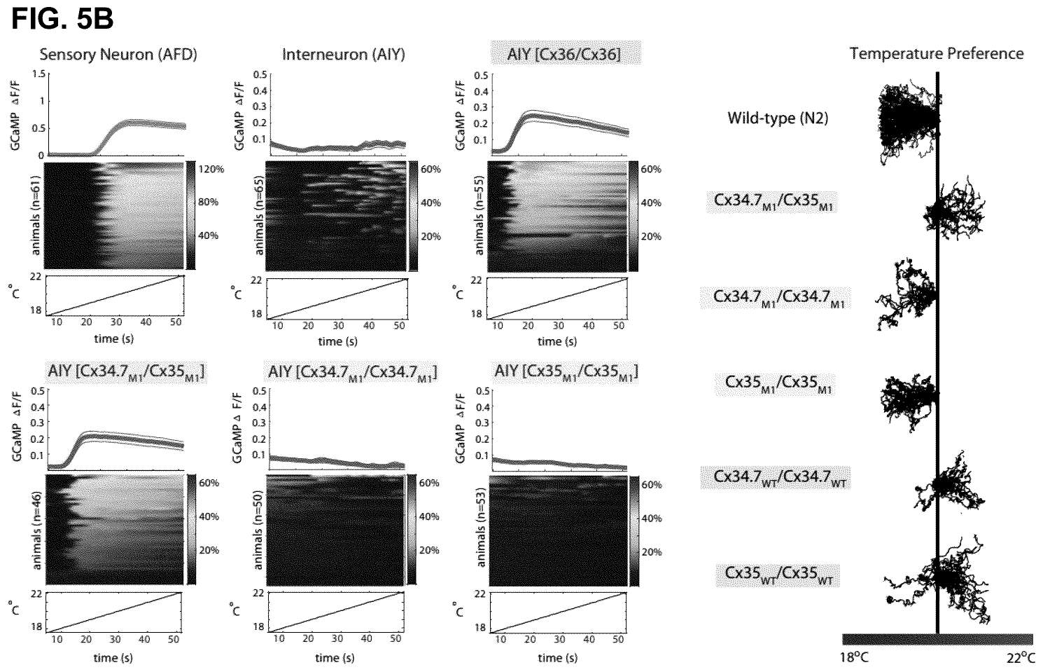

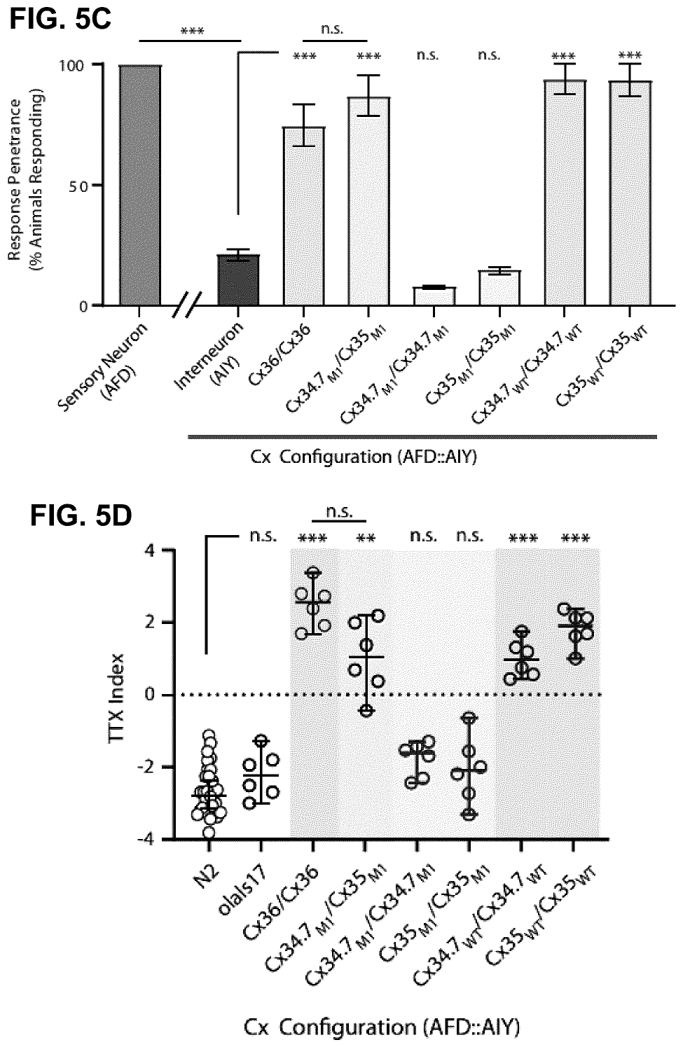

As with other invertebrates, C. elegans do not express connexins. Thus, ectopic expression of vertebrate connexins resulted in formation of electrical synapses that were inert to endogenous gap junction proteins. It was previously shown that ectopic expression of Cx36 could be used to edit the thermotaxis circuit, by bypassing the presynaptic plasticity mechanisms between thermosensory neuron AFD and interneuron AIY that contribute to the learned temperature preference (Hawk J D, et al. (2018) Neuron. 97(2):e4). As such, these circuit-edited animals show a persistent preference for warmer temperatures (FIG. 5A). Therefore, the thermotaxis circuit in C. elegans was used to validate in vivo the utility of the engineered gap junction proteins via de novo formation of electrical synapses (assessed by calcium imaging) and to recode behavior (assessed by quantitative thermotaxis behavior testing).

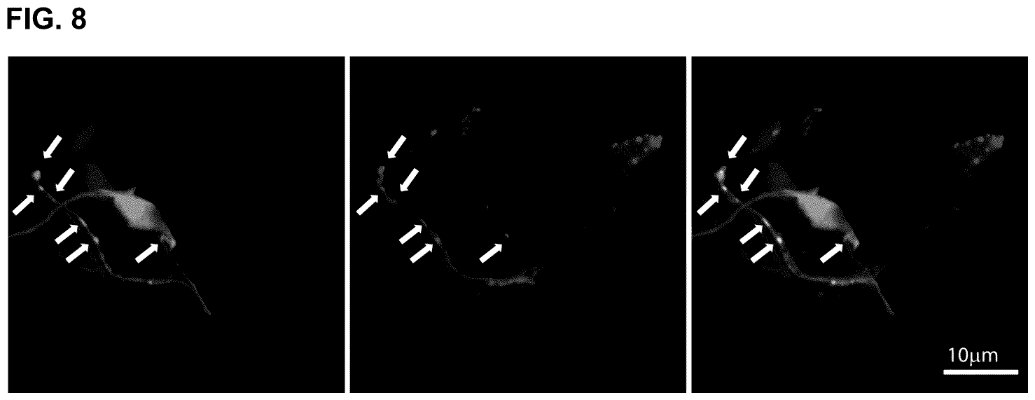

Cx34.7_{M1}/Cx35_{M1} cell-specifically expressed in the AFD/AIY pair and examined whether these proteins were capable of reconstituting functional electrical synapses (FIG. 8 and Table 4). Like Cx36/Cx36, expression of Cx34.7_{M1}/Cx35_{M1} between the AFD/AIY pair resulted in functional coupling between AFD and AIY, as assessed via calcium imaging (FIG. 5B (left) and FIG. 4C; p<0.0005 using Fisher exact test with an FDR correction). These C. elegans constitutively moved towards warmer temperatures when placed on a thermal gradient, again mirroring the animals expressing ectopic Cx36/Cx36 (F_{7,17.91=}84.99; p<0.0001 using Welch one-way ANOVA followed by Dunnett's T3 multiple comparisons; p<0.005; FIG. 5B (right) and FIG. 5D). Expression of Cx34.7_{M1} or Cx35_{M1} in both the AFD and AIY neurons (i.e., Cx34.7_{M1}/Cx34.7_{M1} or Cx35_{M1}/Cx35_{M1}) failed to reconstitute an electrical synapse, though homotypic configuration of both wild type connexins (i.e., Cx34.7_{WT}/Cx34.7_{WT} and Cx35_{WT}/Cx35_{WT}) synchronized the two cells and modulated behavior (FIG. 5B-FIG. 5D, FIG. 8; p<0.0005). Taken together, these findings confirmed that the designer heterotypic gap junction (Cx34.7_{M1}/Cx35_{M1}) exhibited electrical properties sufficient to synchronize the activity of specific neurons in vivo, and rationally recode the behavior. Moreover, these findings confirmed the docking properties predicted for a range of connexin proteins using the disclosed in vitro screen and in silico studies, since both Cx34.7_{M1} and Cx35_{M1} failed to exhibit homotypic docking.

TABLE 4
Listing of C. elegans Strains.

| | Genotype | Source |
|---|---|---|
| N2 | Wild-type | CGC |
| DCR3056 | olaIs17 [Pmod-1::GCaMP6s (25 ng/μL) Pttx-3::mCherry (25 | Hawk et al. |
| | ng/μL) Punc-122::dsRed (40 ng/μL)] | (2018) |
| DCR6604 | olaIs23 [Pgcy-8(800)::caPKC-1B (30 ng/μL), Pgcy- | Hawk et al. |
| | 8(800)::tagRFP (10 ng/μL), Punc-122::RFP (30 ng/μL)]; | (2018) |
| | wyIs629 [Pgcy-8(2kb)::GCaMP6s (30 ng/μL), Pgcy- | |
| | 8(2kb)::mCherry (5 ng/μL), Punc-122:GFP (20 ng/μL)] | |
| DCR5793 | olaIs70 [Pelt-7::GFP (15 ng/μL) + Pgcy-8::CX36::mCh (25 | Hawk et al. |
| | ng/μL)]; olaIs72 [Pelt-7::mCherry (25 ng/μL) + Pttx- | (2018) |
| | 3::CX36::mCh (25 ng/μL)]; olaIs17 [Pmod-1::GCaMP6s (25 | |
| | ng/μL) Pttx-3::mCherry (25 ng/μL) Punc-122::dsRed (40 | |
| | ng/μL)] | |
| DCR5404 | olaEx3219 [Pgcy-8::CX36::mCh (25 ng/μL); PPttx- | Hawk et al. |
| | 3::CX36::mCh (25 ng/μL); Pmyo-3::Red(10 ng/μL)]; olaIs17 | (2018) |
| | [Pmod-1::GCaMP6s (25 ng/μL) PPttx-3::mCherry (25 ng/μL) | |
| | Punc-122::dsRed (40 ng/μL)]; olaIs23 [Pgcy-8(800)::caPKC- | |
| | 1B (30 ng/μL), Pgcy-8(800)::tagRFP (10 ng/μL), Punc- | |
| | 122::RFP (30 ng/μL)] | |
| DCR8225 | olaIs17 [Pmod-1::GCaMP6s (25 ng/μL) PPttx-3::mCherry | Hawk et al. |
| | (25 ng/μL) Punc-122::dsRed (40 ng/μL)]; olaIs23 [Pgcy- | (2018) |
| | 8(800)::caPKC-1B (30 ng/μL), Pgcy-8(800)::tagRFP (10 | |
| | ng/μL), Punc-122::RFP (30 ng/μL)] | |
| DCR8678 | olaEx5223 [Pgcy-8::CX34.7::GFP; Pttx-3::CX34.7::mCh; | Disclosed |
| | Punc-122::GFP (All 25 ng/μL)] | herein |
| DCR8717 | olaEx5255 [Pgcy-8::CX34.7::GFP; Pttx-3::CX34.7::mCh; | Disclosed |
| | Punc-122::GFP (All 25 ng/μL)] | herein |
| DCR8716 | olaEx5254 [Pgcy-8::CX34.7(E214K, E223K)::GFP; Pttx- | Disclosed |
| | 3::CX34.7(E214K, E223K)::mCh; Punc-122::GFP (All 25 | herein |
| | ng/μL)] | |
| DCR8673 | olaEx5218 [Pgcy-8::CX35(K221E)::GFP; Pttx- | Disclosed |
| | 3::CX35(K221E)::mCh; Punc-122::GFP (All 25 ng/μL)] | herein |
| DCR8669 | olaEx5214 [Pgcy-8::CX34.7(E214K, E223K)::GFP; Pttx- | Disclosed |
| | 3::CX35(K221E)::mCh; Punc-122::GFP (All 25 ng/μL)] | herein |
| DCR8684 | olaEx5230 [Pgcy-8::CX35::GFP; Pttx-3::CX35::mCh; Punc- | Disclosed |
| | 122::GFP (All 25 ng/μL)] | herein |
| DCR8719 | olaEx5256 [Pgcy-8::CX35::GFP; Pttx-3::CX35::mCh; Punc- | Disclosed |
| | 122::GFP (All 25 ng/μL)] | herein |
| DCR8715 | olaEx5253 [Pgcy-8::CX34.7(E214K, E223K)::GFP; Pttx- | Disclosed |
| | 3::CX34.7(E214K, E223K)::mCh; Punc-122::GFP (All 25 | herein |
| | ng/μL)] | |
| DCR8674 | olaEx5219 [Pgcy-8::CX35::GFP; Pttx-3::CX35(K221E)::mCh; | Disclosed |
| | Punc-122::GFP (All 25 ng/μL)] | herein |
| DCR8676 | olaEx5221 [Pgcy-8::CX34.7(E214K, E223K)::GFP; Pttx- | Disclosed |
| | 3::CX35(K221E)::mCh; Punc-122::GFP (All 25 ng/μL)] | herein |
| DCR8685 | olaEx5231 [Pgcy-8::CX34.7::GFP; Pttx-3::CX34.7::mCh; | Disclosed |
| | Punc-122::GFP (All 25 ng/μL)] | herein |
| DCR8720 | olaEx5257 [Pgcy-8::CX35::GFP; Pttx-3::CX35::mCh; Punc- | Disclosed |
| | 122::GFP (All 25 ng/μL)] | herein |
| DCR8714 | olaEx5252 [Pgcy-8::CX34.7(E214K, E223K)::GFP; Pttx- | Disclosed |
| | 3::CX34.7(E214K, E223K)::mCh; Punc-122::GFP (All 25 | herein |
| | ng/μL)] | |
| DCR8672 | olaEx5217 [Pgcy-8::CX35(K221E)::GFP; Pttx- | Disclosed |
| | 3::CX35(K221E)::mCh; Punc-122::GFP (All 25 ng/μL)] | herein |
| DCR8677 | olaEx5222 [Pgcy-8::CX34.7(E214K, E223K)::GFP; Pttx- | Disclosed |
| | 3::CX35(K221E)::mCh; Punc-122::GFP (All 25 ng/μL)] | herein |
| DCR8675 | olaEx5220 [Pgcy-8::CX34.7::GFP; Pttx-3::CX34.7::mCh; Pelt- | Disclosed |
| | 7::NLS::mCh (All 25 ng/μL)]; olaIs17 [Pmod-1::GCaMP6s (25 | herein |
| | ng/μL) PPttx-3::mCherry (25 ng/μL) Punc-122::dsRed (40 | |
| | ng/μL)]; olaIs23 [Pgcy-8(800)::caPKC-1B (30 ng/μL), Pgcy- | |
| | 8(800)::tagRFP (10 ng/μL), Punc-122::RFP (30 ng/μL)] | |
| DCR8776 | olaEx5287 [Pgcy-8::CX35::GFP; Pttx-3::CX35::mCh; Punc- | Disclosed |
| | 122::GFP (All 25 ng/μL)]; olaIs17 [Pmod-1::GCaMP6s (25 | herein |
| | ng/μL) PPttx-3::mCherry (25 ng/μL) Punc-122::dsRed (40 | |
| | ng/μL)]; olaIs23 [Pgcy-8(800)::caPKC-1B (30 ng/μL), Pgcy- | |
| | 8(800)::tagRFP (10 ng/μL), Punc-122::RFP (30 ng/μL)] | |
| DCR8777 | olaEx5288 [Pgcy-8::CX34.7(E214K, E223K)::GFP; Pttx- | Disclosed |
| | 3::CX34.7(E214K, E223K)::mCh; Punc-122::GFP (All 25 | herein |
| | ng/μL)]; olaIs17 [Pmod-1::GCaMP6s (25 ng/μL) PPttx- | |
| | 3::mCherry (25 ng/μL) Punc-122::dsRed (40 ng/μL)]; olaIs23 | |
| | [Pgcy-8(800)::caPKC-1B (30 ng/μL), Pgcy-8(800)::tagRFP (10 | |
| | ng/μL), Punc-122::RFP (30 ng/μL)] | |
| DCR8671 | olaEx5216 [Pgcy-8::CX35(K221E)::GFP; Pttx- | Disclosed |
| | 3::CX35(K221E)::mCh; Pelt-7::NLS::mCh (All 25 ng/μL)]; | herein |
| | olaIs17 [Pmod-1::GCaMP6s (25 ng/μL) PPttx-3::mCherry (25 | |
| | ng/μL) Punc-122::dsRed (40 ng/μL)]; olaIs23 [Pgcy- | |
| | 8(800)::caPKC-1B (30 ng/μL), Pgcy-8(800)::tagRFP (10 | |
| | ng/μL), Punc-122::RFP (30 ng/μL)] | |
| DCR8670 | olaEx5215 [Pgcy-8::CX34.7(E214K, E223K)::GFP; Pttx- | Disclosed |
| | 3::CX35(K221E)::mCh; Pelt-7::NLS::mCh (All 25 ng/μL)]; | herein |
| | olaIs17 [Pmod-1::GCaMP6s (25 ng/μL) PPttx-3::mCherry (25 | |
| | ng/μL) Punc-122::dsRed (40 ng/μL)]; olaIs23 [Pgcy- | |
| | 8(800)::caPKC-1B (30 ng/μL), Pgcy-8(800)::tagRFP (10 | |
| | ng/μL), Punc-122::RFP (30 ng/μL)] | |

### Example 4

### Synthetic Cx34.7M1/Cx35M1 Enhanced Synchrony within a Mammalian Neural Circuit

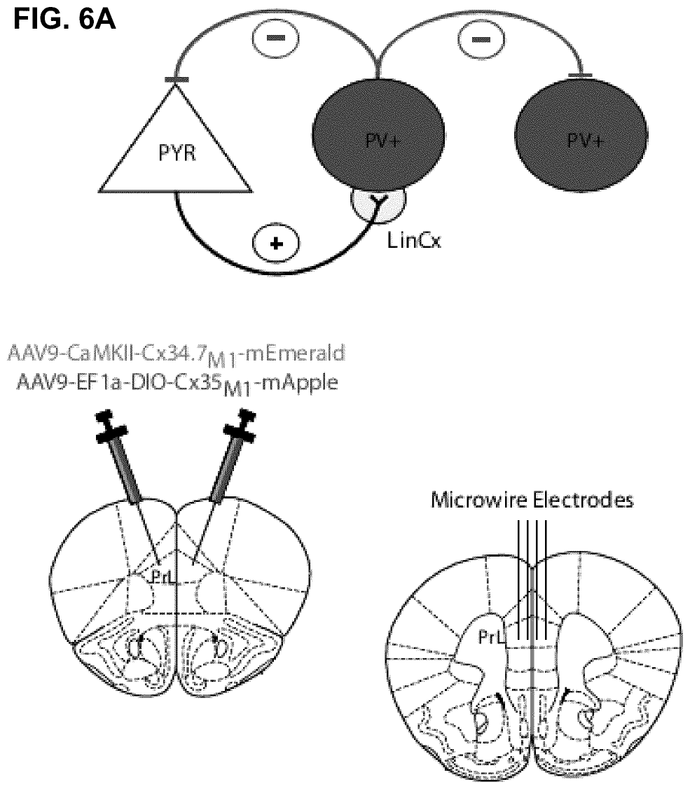

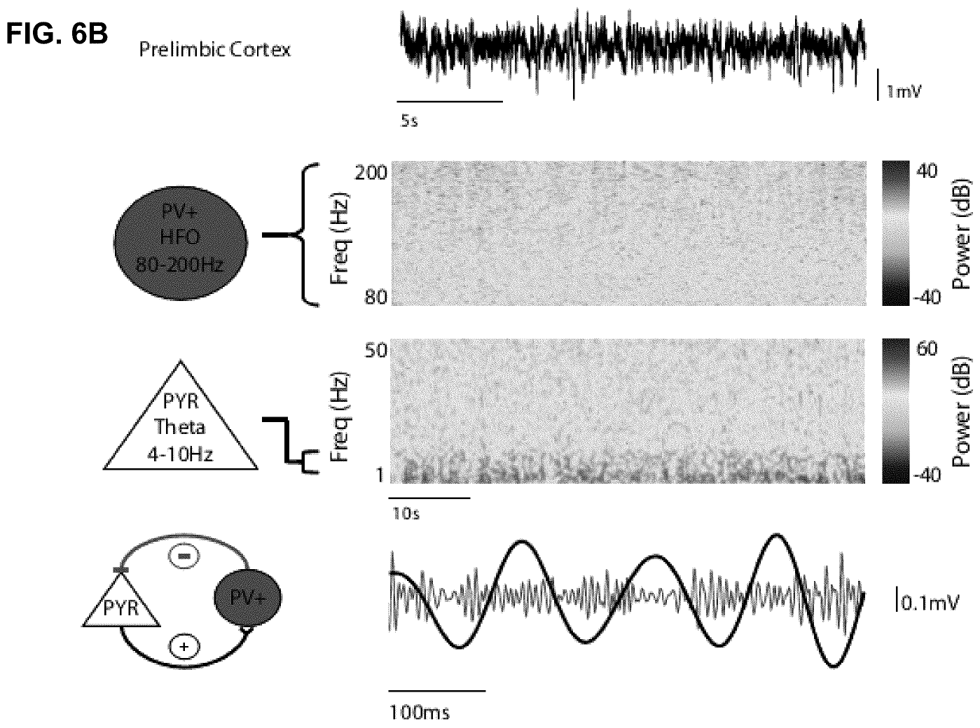

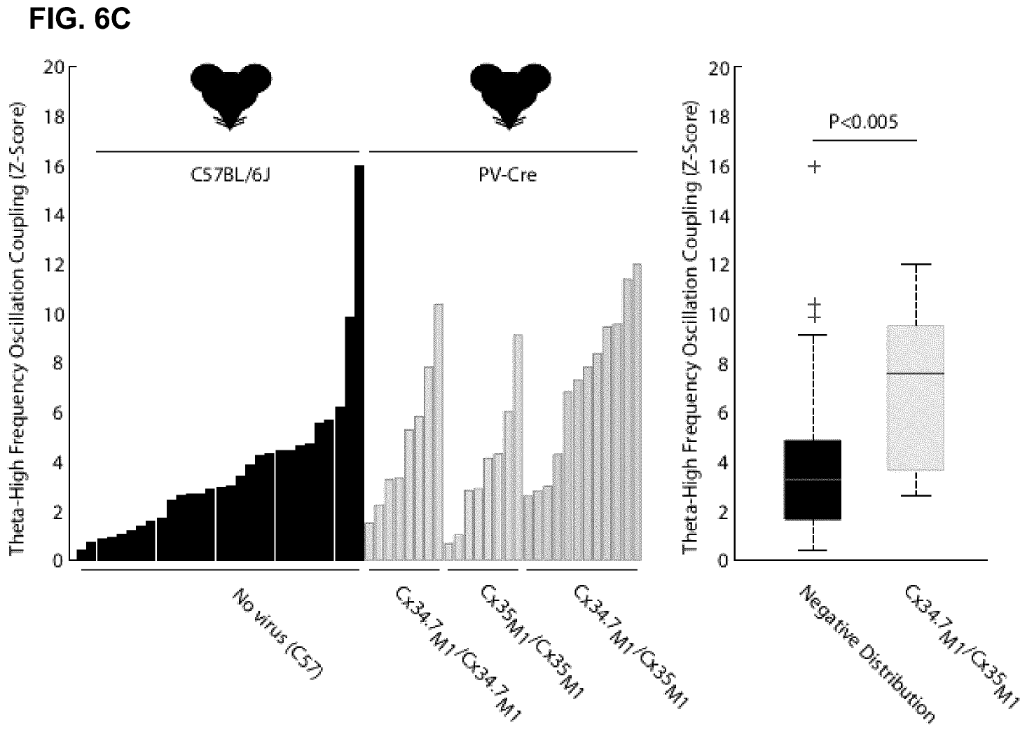

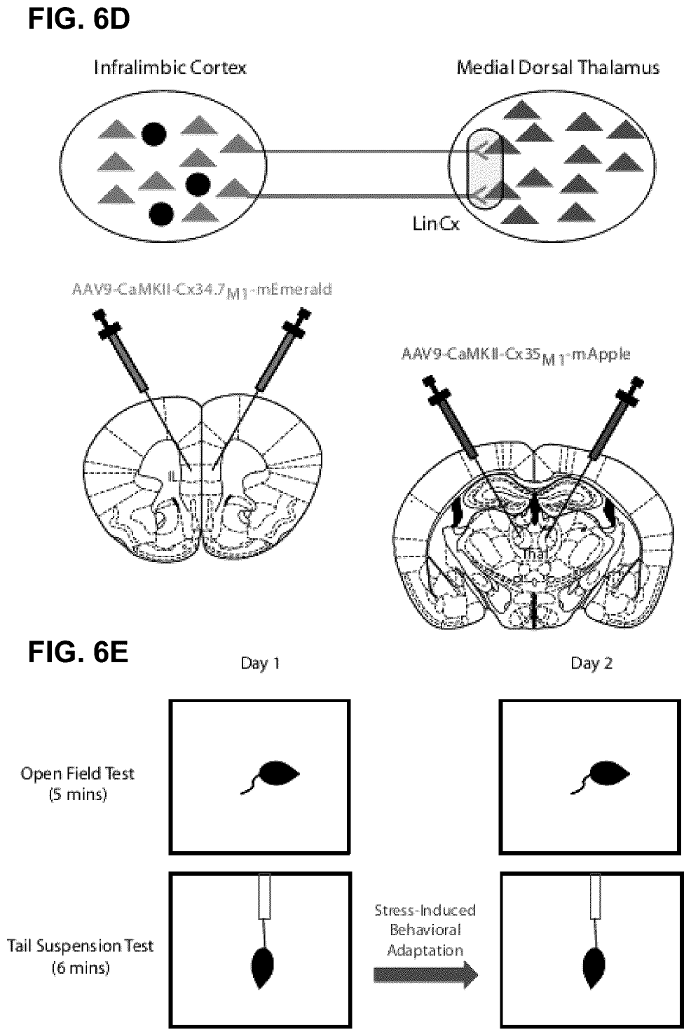

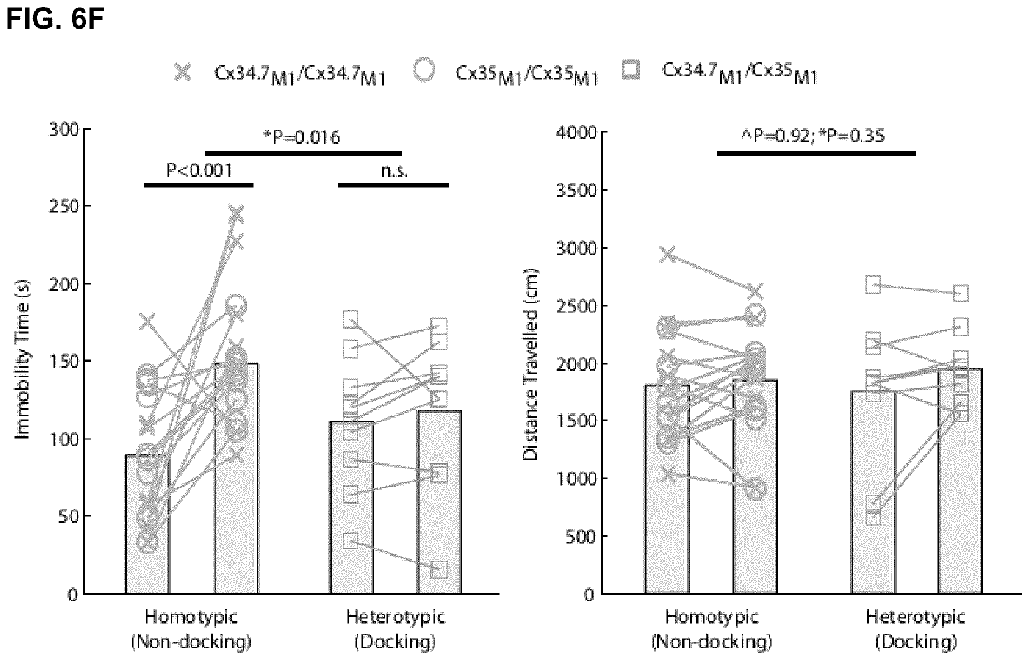

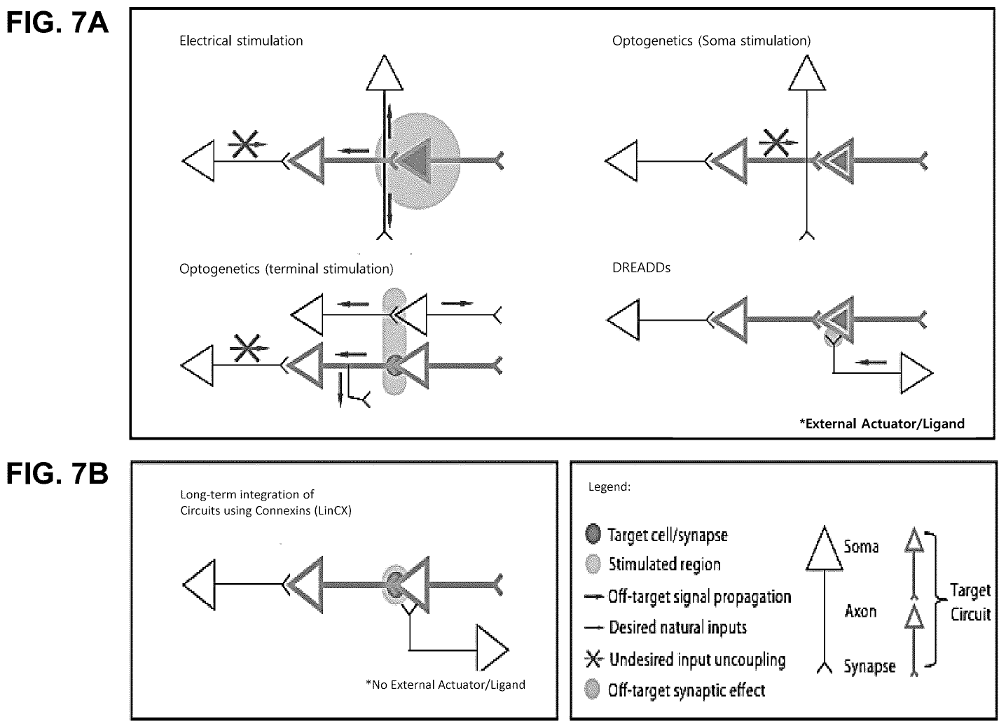

Having established the docking selectivity and in vivo functionality of the Cx34.7_{M1}/CX35_{M1} pair, whether the synthetic electrical synapse that the pair forms can be used to modulate neural circuitry in a species with endogenous connexins was examined. Thus, editing a neurocircuit in the mammalian brain composed of two distinct cell types was undertaken. To do this, mice, which are highly amenable to cell-type specific targeting, were used. Excitatory pyramidal neurons (PYR) and parvalbumin expressing fast-spiking interneurons (PV+) can form microcircuits whereby PYR neurons excite PV+ neurons, which in turn inhibit PYR neurons (FIG. 6A (top)). This PYR↔PV+ neural circuit has been well characterized in the hippocampus, where PYR neurons show activity coupled to the phase of theta frequency (4 Hz-10 Hz) oscillations during spatial exploration (Siapas A G, et al. (2005) Neuron. 46(1):141-151) and PV+ neuron activity correlates with gamma frequency oscillations (30 Hz-80 Hz) (Fuchs E C, et al. (2007) Neuron. 53(4):591-604). Critically, the activity of this PYR-PV+ microcircuit is reflected by the millisecond resolved synchrony between the phase of theta oscillations and the amplitude of gamma oscillations (Wulff P, et al. (2009) Proc Natl Acad Sci USA. 106(9):3561-3566).

PYR↔PV+ microcircuits are also observed in the prefrontal cortex with slightly different neurophysiological properties (Sohal V S, et al. (2009) Nature. 459(7247):698-702). Like the cellular dynamics observed in the hippocampus, prefrontal cortex PYR neurons phase couple to locally recorded theta oscillations (Dzirasa K, et al. (2010). J Neurosci. 30(48):16314-16323). On the other hand, in prefrontal cortex, PV+ neurons best couple to the phase and amplitude of local high frequency oscillations (80 Hz-200 Hz) (Yao Y, et al. (2020) Front Cell Neurosci. 14:610741). Thus, the coupling between phase of prefrontal cortex theta oscillations and the amplitude of prefrontal cortex high frequency oscillations was quantified as a proxy of prefrontal cortex PRY↔PV+ microcircuit activity. The synthetic Cx34.7_{M1}/Cx35_{M1} synapse was expressed at the PRY→PV+, anticipating that this manipulation would enhance the millisecond-timed coupling between prefrontal cortex theta and high frequency oscillations.

Specifically, an adeno-associated virus (AAV9-CaMKII-Cx34.7_{M1}-mEmerald) was developed to target Cx34.7_{M1} to PYR neurons, and another virus (AAV9-Ef1α-DIO-Cx35_{M1}-mApple) was developed to target Cx35_{M1} to cells expressing Cre-recombinase. PV-Cre mice were then co-infected with both viruses bilaterally such that Cx34.7_{M1} was expressed in PYR neurons and Cx35_{M1} was pressed in PV+ neurons in the PrL (i.e., PYR→PV+ modulation given Cx34.74→Cx35 rectification of the hemichannel pair; FIG. 6A (bottom)). A control distribution of non-edited mice consisted of three groups: (i) C57BL/6J mice that had not been infected with virus (n=29), (ii) PV-Cre mice co-infected with AAV9-CaMKII-Cx34.7_{M1}-mEmerald and AAV9-Ef1α-DIO-Cx34.7_{M1}-mEmerald, which expressed Cx34.7_{M1} in a non-docking configuration in both cell types (n=8), and (iii) PV-Cre mice co-infected with AAV9-CaMKII-Cx35_{M1}-mApple and AAV9-Ef1α-DIO-Cx35_{M1}-mApple to express Cx35_{M1} in both cell types (n=8). All mice were implanted with microwire electrodes bilaterally in prelimbic cortex (PrL, a subdivision of prefrontal cortex in mice), and neural oscillation activity was recorded while mice explored an open field (FIG. 6A and FIG. 6B).

To determine the coupling between theta and high frequency oscillations (80 Hz-200 Hz), local field potential activity in these two frequency bands was isolated (FIG. 6B). Phase-amplitude relationships were then determined using the established modulation index (z-score), which quantified the statistical likelihood that measured relationships between two oscillations would be observed by chance (Canolty R T, et al. (2006) Science. 313(5793):1626-1628). Using this approach, significant theta-high frequency oscillation coupling from at least one recording site in 49/57 implanted mice was found (FIG. 6C). Moreover, theta-high frequency oscillation coupling was significantly higher in the mice expressing the synthetic synapse when compared to the pooled group of unedited mice (U=1161; p=0.0025 using one tailed rank-sum test). A post-hoc analysis also found no differences in theta-high frequency coupling between mice expressing Cx34.7_{M1}/Cx34.7_{M1} or mice expressing Cx35_{M1}/Cx35_{M1} across both cell types compared to uninfected C57BL/6j control mice (U=514; p=0.18 using two tailed rank-sum test for Cx34.7_{M1}/Cx34.7_{M1} and U=544; p=0.81 using two tailed rank-sum test for Cx35_{M1}/Cx35_{M1}). Thus, it was found that expression of the synthetic synapse was sufficient to enhance millisecond timed synchrony within a circuit defined by two precise cell types in mammals. The findings from the homotypic controls confirmed the non-homotypic docking selectivity.

Finally, whether the synthetic synapse could be used to edit the function of a long-range circuit and modify behavior was examined. The Infralimbic cortex (IL, another anatomical subdivision of medial prefrontal cortex in mice) and medial dorsal thalamus (MD) form a monosynaptic circuit in mice. To target this circuit, BALB/cj mice were infected with AAV9-CaMKII-Cx34.7_{M1}-mEmerald bilaterally in IL. Three weeks later, the same BALB/cj mice were infected with AAV9-CaMKII-Cx35_{M1}-mApple bilaterally in MD (i.e., IL→MD modulation given Cx34.74→Cx35 rectification of this hemichannel pair; FIG. 6D; n=10 mice). A negative control group of mice was injected with either AAV9-CaMKII-Cx34.7_{M1}-mEmerald or AAV9-CaMKII-Cx35_{M1}-mApple in both regions to express synthetic hemichannels across the IL→MD circuit in homotypic non-docking configurations. All mice were subjected to testing in an open field, immediately followed a tail suspension test (FIG. 6E).

The tail suspension test is a classic assay that measures the behavioral response of mice to an inescapable negative experience in which they are suspended upside down by their tail (Steru L, et al. (1985) Psychopharmacology (Berl). 85(3):367-370). Prior exposure to stress diminishes behavioral responses during the assay (Iniguez S D, et al. (2016) Neurobiol Stress. 5:54-64), and the assay itself induces a robust stress response (Ide S, et al. (2010) Neuropharmacology. 58(1):241-247). These two features of the test were previously exploited to demonstrate that repeated exposure to the tail suspension test increased immobility during subsequent testing. This stress induced behavioral adaptation was correlated with functional changes in infralimbic cortex and medial dorsal thalamus. Furthermore, exogenous stimulation of the IL→MD circuit in a manner that recapitulated the normal synchrony in the circuit reduced behavioral adaptation to acute stress (Carlson D, et al. (2017) Biol Psychiatry. 82(12):904-913). Critically, no behavioral differences were observed when exploratory behavior was assayed immediately prior to the tail suspension sessions; demonstrating that the behavioral adaptation was specific to the stressful context (Carlson D, et al. (2017) Biol Psychiatry. 82(12):904-913).

Here, this behavioral paradigm was employed to test whether expression of the synthetic Cx34.7_{M1}/Cx35_{M1} electrical synapses could alter the stress adaptation. Whether the synthetic synapse would synchronize activity between the IL and MD thal neurons, thereby preventing the immobility adaptation previously seen on day 2 of the TST, was examined. Interestingly, mice expressing the synthetic synapse across the IL-MD circuit did not show behavioral adaptation in response to repeat tail suspension testing (F_{1,33}=13.97, p=0.003 for Group×Day interaction effect using mixed effects model ANOVA; t₁₅=0.46; p=0.63 for post-hoc testing using two-tailed paired t-test for Cx34.7_{M1}/Cx35_{M1} mice across days; FIG. 6F). On the other hand, increases in immobility were observed in the negative control group (t₁₈=4.63; p=2.1×10⁻⁴ for post-hoc testing using two-tailed paired t-test for pooled group of Cx34.7_{M1}/CX34.7_{M1} and Cx35_{M1}/Cx35_{M1} mice across days). Post-hoc analysis revealed increases in immobility in both the Cx34.7_{M1}/CX34.7_{M1} and CX34.7_{M1}/CX34.7_{M1} groups expressing synthetic hemichannels in non-docking configurations. Thus, the expression of the functional synthetic synapse across the IL→MD circuit prevented behavioral adaptation to the tail suspension test. No differences in open field exploration were observed between the mice that expressed Cx34.7_{M1}/Cx35_{M1} and control mice expressing the hemichannels in non-docking configurations (F_{1,33}=0.15, p=0.70 for Group effect; F_{1,33}=4.02, p=0.053 for Group×Day interaction effect using mixed effects model ANOVA), demonstrating that editing IL→MD only changed behavior in the stressful assay (FIG. 6F). Critically, mice showed higher exploration in the open field across testing sessions demonstrating that the increase in immobility induced by the tail suspension test was context specific (F_{1,33}=10.63, p=0.0026 for Day effect), again providing additional evidence that the behavioral adaptation induced by acute tail suspension stress was context specific.

### Summary of Experiments

These studies characterize a novel approach named “Long-term integration of Circuits using Connexins” or LinCx that can be used to rationally edit brain circuits. This approach employs an engineered connexin hemichannel pair capable of heterotypic, but not homotypic, docking. When expressed in two adjacent cells, these hemichannels compose a heterotypic electrical synapse, facilitating the transfer of activity between them. Here, LinCx was engineered using two connexin proteins found in Morone americana (white perch fish)→connexin 34.7 (Cx34.7) and connexin 35 (Cx35). These connexins are expressed in the CNS at pre-and post-synaptic terminals in Morone americana and show rectification in the Cx34.7 to Cx35 direction (Rash J E, et al. (2013). Neuron. 79(5):957-969). Here, the engineered proteins had a preferential direction for facilitating the transfer of electrical activity and did not dock homotypically. This latter feature ensured that even when a hemichannel is expressed across a cell type, those cells would not form electrical synapses between themselves (FIG. 1A).

Thus, the disclosed LinCx system can be deployed in higher order animals, which have many more cells of each given cell type, to target distinct neural circuits. This novel feature was demonstrated in mice by editing a cortical microcircuit composed of two genetically defined cell types. To facilitate such strong docking specificity, a two-part strategy was employed to engineer a Cx34.7 and Cx35 pair that solely exhibited heterotypic docking. First, a two-part strategy was used to engineer a Cx34.7 and Cx35 pair that solely exhibited heterotypic docking. Then, a library of Cx34.7 and Cx35 mutants was created and was based on sites previously shown to confer and/or alter docking specificity as well as those sites implicated in docking based on homology modeling from the structures of Cx26 (Maeda S, et al. (2009) Nature. 458(7238):597-602), (Bennett B C, et al. (2016) Nat Commun. 7:8770). These mutants were subjected to the rapid screening approach to identify the mutations that disrupted homotypic docking for each Cx protein. Next, non-homotypic Cx34.7 and Cx35 mutant proteins were combinatorially screened against each other to identify pairs that retained their heterotypic docking character. Second, a computational model of Cx protein docking was developed. This computational model was used to probe the docking properties of the mutants identified in the initial FETCH screen and was used to design new Cx mutants in silico. At each step of the in silico design processes, the homotypic and heterotypic docking properties of the designed mutants were validated by direct assay using the in vitro docking screen. Finally, all the mechanistic understanding of Cx docking interactions gained through this two-pronged approach were integrated to engineer Cx34.7 and Cx35 mutants that show heterotypic, but not homotypic docking. This exclusively heterotypic docking profile has never been observed for pairs of connexin hemichannels. The functionality of the Cx34.7 and Cx35 mutant pair and their unique docking configuration was validated in vivo. Specifically, in C. elegans, it was demonstrated that the expression of the mutant pair in two distinct cells successfully edited the circuit and modulated behavior.

The integrated engineering approach utilized to develop LinCx (see FIG. 2) can now be deployed to develop a toolbox of connexin protein pairs that exhibit selective docking properties. Future work may also yield novel hemichannel pairs with customized conductance properties, mirroring approaches applied to modify the conductance of invertebrate electrical synapses (Shui Y, et al. (2020). Sci Adv. 6(27):eabb3076). Thus, the work described herein made it possible to deploy multiple LinCx pairs in the same animal to simultaneously edit multiple circuits and ultimately regulate brain function. The translational potential for LinCx was further optimized by engineering Cx34.7 and Cx35 to not only disrupt their homotypic docking, but also to disrupt their heterotypic hemichannel docking with other connexin proteins endogenous to the human brain (e.g., Cx36, Cx43, and Cx45).

Given the sequence homology/identity of ELs across mammals (Table 5), the LinCx can be broadly applicable to other preclinical model organisms including rodents and non-human primates. To demonstrate this wide applicability, this unique feature of LinCx was validated directly in mice, where LinCx successfully edited dynamics that putatively reflect a microcircuit composed of two genetically distinct cell types. Moreover, since LinCx enhanced coupling of neural activity to the phase of theta oscillations (i.e., periodicity of 100 ms-250 ms), the electrical recordings established the millisecond temporal precision of LinCx. Critically, LinCx can also be deployed alongside other well established preclinical modulation approaches including optogenetics and DREADDs, enabling broad manipulation of brain networks across multiple scales of spatial, temporal, and context resolution concurrently.

TABLE 5
Homology of Connexin Proteins Between Humans and Several Species.

| | | SEQ |
|---|---|---|
| Species | Sequence | ID NO: |
| Cx36 EL2 | 227-GLYECNRYPCIKEVECYVSRPTEKTVFLVF-256 | 1 |
| Homo sapiens | | |
| Cx36 EL2 | 227-GLYECNRYPCIKEVECYVSRPTEKTVFLVF-256 | 2 |
| Mus musculus | | |
| Cx36 EL2 | 227-GLYECNRYPCIKEVECYVSRPTEKTVFLVF-256 | 3 |
| Macaca mulatta | | |
| Cx36 EL2 | 289-GLYECNRYPCIKEVECYVSRPTEKTVFLVF-318 | 4 |
| Callithrix jacchus | | |
| Cx36 EL2 | 196-AIFECDRYPCVKEVECYVSRPTEKSVFLVF-225 | 5 |
| Taeniopygia guttat | | |
| Cx36 EL2 | 210-AVYECDRYPCIKDVECYVSRPTEKTVFLVF-239 | 6 |
| Danio rerio (Cx35) | | |
| Cx43 EL2 | 171-LIQWYIYGFSLSAVYTCKRDPCPHQVDCFLSRPTEK-206 | 7 |
| Homo sapiens | | |
| Cx43 EL2 | 171-LIQWYIYGFSLSAVYTCKRDPCPHQVDCFLSRPTEK-206 | 8 |
| Mus musculus | | |
| Cx43 EL2 | 171-LIQWYIYGFSLSAVYTCKRDPCPHQVDCFLSRPTEK-206 | 9 |
| Macaca mulatta | | |
| Cx43 EL2 | 171-LIQWYIYGFSLSAVYTCKRDPCPHQVDCFLSRPTEK-206 | 10 |
| Callithrix jacchus | | |
| Cx43 EL2 | 171-LIQWYIYGFSLNAIYTCERDPCPHRVDCFLSRPTEK-206 | 11 |
| Taeniopygia guttata | | |
| Cx43 EL2 | 171-VIQWYLYGFSLSAVYTCERTPCPHRVDCFLSRPTEK-206 | 12 |
| Danio rerio | | |
| Cx45 EL2 | 200-GFQVHPFYVCSRLPCPHKIDCFI-222 | 13 |
| Homo sapiens | | |
| Cx45 EL2 | 200-GFQVHPFYVCSRLPCPHKIDCFI-222 | 14 |
| Mus musculus | | |
| Cx45 EL2 | 200-GFQVHPFYVCSRLPCPHKIDCFI-222 | 15 |
| Macaca mulatta | | |
| Cx45 EL2 | 200-GFQVHPFYVCSRLPCPHKIDCFI-222 | 16 |
| Callithrix jacchus | | |
| Cx45 EL2 | 198-RFEVSPSYVCSRSPCPHTVDCFV-220 | 17 |
| Taeniopygia guttata | | |
| Cx45 EL2 | 196-GFEVAPSYVCTRSPCPHTVDCFV-218 | 18 |
| Danio rerio | | |

Table 5 show the sequence alignment of several connexin proteins predicted extracellular loop 2, related to FIG. 6. Predicted EL2 regions of Cx36 (GJD2), Cx43 (GJA1), and Cx45 (GJC1) for humans and several species broadly utilized in neuroscience research, related to FIG. 6A-FIG. 6F. Identical residues are shown in black, residues that are variable are highlighted with red text. Residues of Cx36 that align to the interaction motif of Cx34.7 and Cx35 are indicated by blue underline. Note zebrafish (Danio rerio) do not have a Cx36 gene, thus the closest homolog, Cx34.7, was used for comparison.

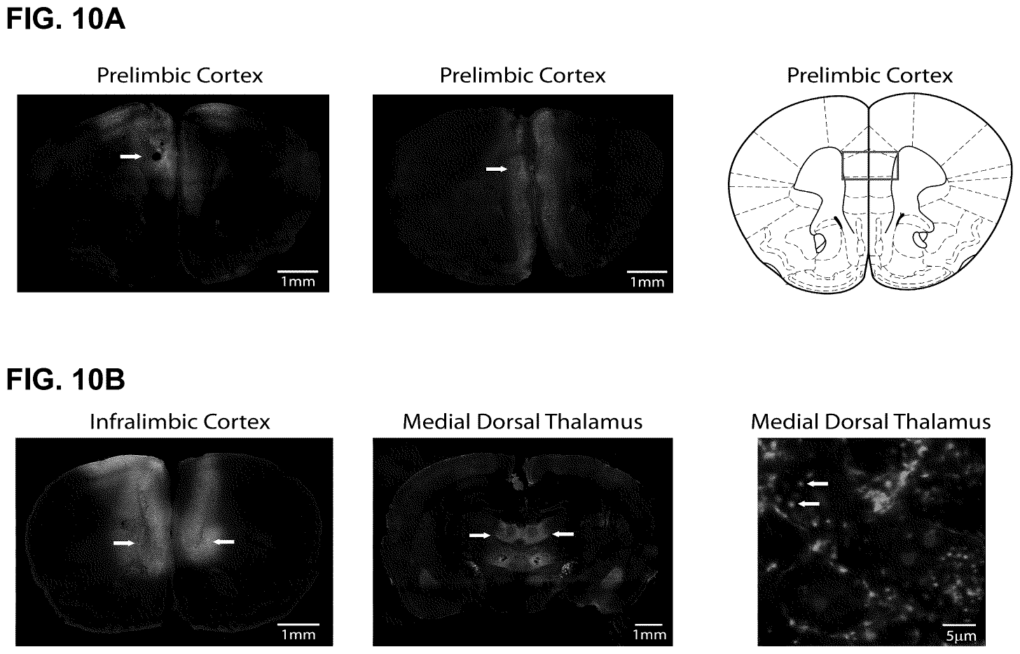

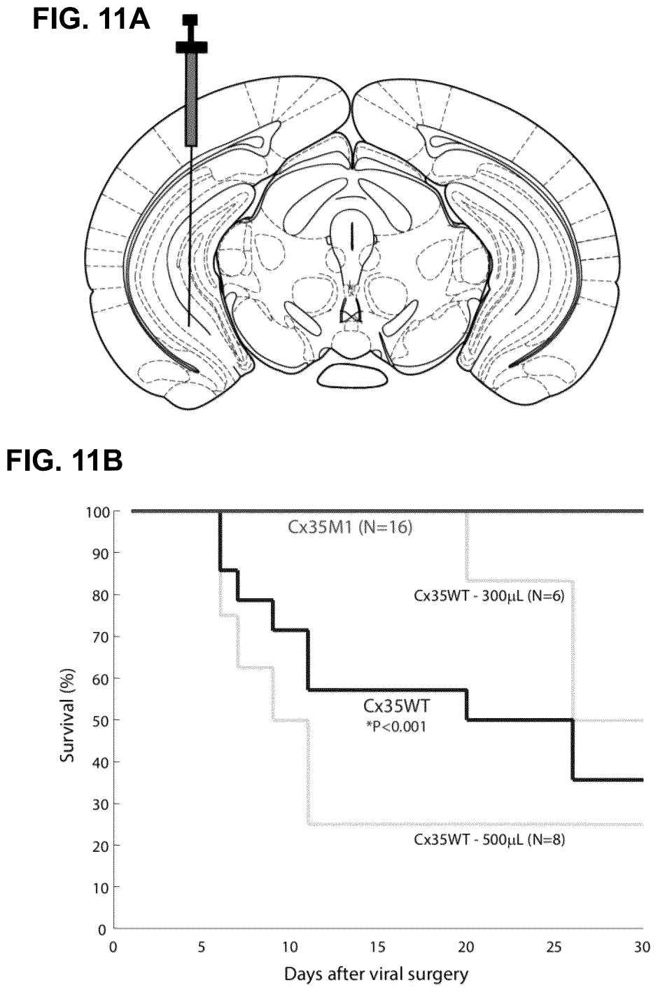

Within brain circuits, space is operationalized as the physical boundaries of individual cells, and time is operationalized as the sub-millisecond level electrical changes of those cells. Though many classic electrical stimulation approaches exhibit high temporal precision in their targeting, these techniques often stimulate volumes of brain tissue that include many brain cell-types and local axonal fibers of passage (FIG. 10A-FIG. 10B; electrical stimulation). Preclinical approaches such as optogenetics provide a substantial improvement regarding spatial targeting by enabling the selective stimulation of specific cell bodies (based on their genetic identities). Moreover, optogenetics modulates cells via temporally precise light pulses, maintaining the temporal precision of electrical stimulation. Nevertheless, both electrical and optogenetics stimulation bear substantial potential to override circuit computations, which integrate space and time across precise cell types, since these approaches are typically utilized under conditions that modulate the activity of many neurons concurrently, and outside of the activity context of their inputs (FIG. 10A-FIG. 10B; optogenetics—soma stimulation).

Over the last decade, several strategies have been employed across myriad studies to enhance the context precision of circuit targeting. One such strategy is based on selectively modulating projection neurons, whereby the inputs at a targeted brain site are selectively activated (FIG. 10A-FIG. 10B; optogenetics—projection stimulation) (Deisseroth K, et al. (2011) Nat Methods. 8(1):26-29). By directly modulating presynaptic nerve fibers, investigators can regulate the context (presynaptic neurotransmitter release) that drives the target cellular response. Nevertheless, this approach has the potential to change physiological variables that define the context of the presynaptic neuron (and thus the circuit). For example, terminal stimulation can activate axonal collaterals of the presynaptic neuron thereby decreasing the spatial precision of circuit targeting, or it can induce retrograde activation of the presynaptic cell in a manner that disrupts the activation context of that neuron relative to its own inputs. This approach can also drive the activation of non-target circuits since inputs from a brain region can synapse onto multiple distinct cell types.

Other strategies utilized to enhance the context precision of circuit targeting include the stable step function opsins (SSFOs) (Yizhar O, et al. (2011) Nature. 477(7363):171-178) and designer receptors exclusively activated by designed drugs (DREADDs) (Armbruster B N, et al. (2007) Proc Natl Acad Sci USA. 104(12):5163-5168), which function to increase the resting membrane potential of target cells. SSFOs and DREADDs maintain the cell type specific spatial precision characteristic of initial optogenetic targeting approaches. Moreover, because cells are rendered more likely to fire in response to their input signals under optimal conditions, these approaches provide improved temporal and context precision. Nevertheless, SSFOs and DREADDs can render target neurons more responsive to all their excitatory inputs (including those from non-targeted circuits), raising the potential of circuit-level off-target effects (FIG. 10A-FIG. 10B; DREADDs). Thus, there is still demand for neuromodulation approaches that function within the spatial, temporal, and context constraints that together define brain circuit operation.

Another strategy to address limitations in context precision is to deliver stimulation within a closed loop framework. In this framework, neural activity is modulated based the ongoing activity in the brain. For example, stimulating hippocampus at the peak vs. trough of the endogenous theta oscillatory cycle differentially impacts spatial memory (Siegle J H, et al. (2014). Elife. 3:e03061). In prior work exploring the circuitry underlying stress induced behavioral adaptation, neurons in medial dorsal thalamus were stimulated while mice were engaged in a tail suspension testing (Carlson D, et al. (2017) Biol Psychiatry. 82(12):904-913). When neurons were activated based on ongoing oscillatory activity in infralimbic cortex during the test (i.e., closed loop circuit stimulation), mice showed decreased immobility. Conversely, when these same cells were stimulated using two distinct temporal patterns that were untimed to activity in infralimbic cortex, either increased behavioral immobility or no behavioral effect at all were observed. Additionally, no behavioral effect was observed when the terminals of infralimbic cortex neurons in medial dorsal thalamus was stimulated. Together, these observations provided evidence that context precision serves as another critical axis of circuit operation in the brain that is orthogonal to both spatial and temporal precision. Moreover, these findings highlighted an ideal circuit and behavioral paradigm to test this new neuromodulation approach that was designed to preserve the spatial, temporal, and context precision of neural circuits.

Here, LinCx modulation of the IL→MD circuit decreased immobility during repeat tail suspension testing. This was a behavioral outcome that could only previously be achieved through closed loop stimulation. Interestingly, mice expressing LinCx exhibited normal behavior on the first day of tail suspension testing (t₃₃=1.24; p=0.22 compared to non-edited mice using post-hoc testing with unpaired two-tailed t-test). This indicated that rather than driving acute stress behavior per se', LinCx actually enhanced the compensatory function of endogenous IL→MD circuitry, ultimately suppressing behavioral adaptation in response to acute stress. As such, the findings disclosed herein indicate that LinCx likely enhanced circuits within their physiological range, rather than driving them into supraphysiological states. This observation was supported by prelimbic cortex micro-circuit editing findings that revealed that the majority of LinCx edited mice exhibited cross-frequency coupling that was less than the upper bound of the unedited mice.

Like established protein-based modulation tools such as optogenetics and DREADDs, LinCx can be targeted to precise cell types. LinCx builds upon these technologies by enabling each hemichannel to be expressed in a different cell type. The hemichannels expressed by these two distinct cell types then integrate in vivo to form an electrical synapse. As such, LinCx offers unprecedented spatial precision compared to optogenetics and DREADDs in that it enables targeting of one of the specific spatial features that constrains circuits (e.g., the structural integration of two distinct cell types). Importantly, no electrical synapses are constituted between distinct cells of the same cell type since both hemichannels' abilities to dock homotypically was disrupted (FIG. 1A).

LinCx is also designed to optimize the context precision of neuromodulation. Because this electrical synapse rectifies and it only forms between the target pre- and post-synaptic neuron, LinCx constrained the modulation of each individual post-synaptic neuron by the endogenous activity of its genetically defined pre-synaptic partner. This feature yielded a level of temporal precision that mirrors the precision of endogenous brain activity. Moreover, this feature indicates that LinCx only potentiates circuit activity within the broader brain state context for which those circuits are typically engaged (i.e., the presynaptic neuron remains under the normal control of its own inputs). Finally, unlike established modulation approaches, LinCx does not require an exogenous actuator such as light, electricity, or an inert pharmacological compound. Rather, LinCx utilizes endogenous brain activity to modulate target neurons, yielding a tool for precise circuit editing.

Direct stimulation of the brain is a well-established treatment for neurological and psychiatric disorders. For example, electrical convulsive therapy (ECT), which delivers energy to the whole brain, has remained the most effective treatment for major depressive disorder for nearly a century (Pagnin D, et al. (2004) J ECT. 20(1):13-20). Deep brain stimulation (DBS) of the sub thalamic nucleus is a widely utilized therapeutic for Parkinson's disease (Liu Y, et al. (2014) J Neurosurg. 121(3):709-718). Nevertheless, both these modalities have important spatiotemporal constraints. ECT can induce short term cognitive dysfunction, likely due the spatially untargeted nature of energy delivery to the brain, ultimately limiting its clinical use. On the other hand, DBS of the subthalamic nucleus has limited impact on the devastating emotional and cognitive symptoms that accompany Parkinson's disease, likely due to the brain region targeting selectivity (Liu Y, et al. (2014) J Neurosurg. 121(3):709-718). While non-invasive techniques such as transcranial magnetic and focused ultrasound hold future promise for expanding the therapeutic landscape for brain disorders due to their increased clinical accessibility (these approaches do not require anesthesia/brain surgery), there continues to be demand for novel therapeutic approaches that modulate brain activity within both the spatial and temporal constrains of brain circuit activity.

There is great potential for LinCx as a tool to probe the causal relationship between brain circuit function and behavior, and as a therapeutic approach to ameliorate human neuropsychiatric disorders. Moreover, there are many potential applications for LinCx technology that extend beyond the central nervous system including, for example, as a treatment for Myasthenia Gravis, arrhythmias, inflammation, a range of disorders associated with gastrointestinal dysfunction, or to ameliorate chronic pain.

1. An isolated nucleic acid molecule, comprising: a nucleic acid sequence encoding a designer Cx34.7 connexin protein, wherein the designer Cx34.7 connexin protein comprises the Cx34.7 of Morone americana having a substitution at encoded amino acid 214 and at amino acid 223.

2. (canceled)

3. The isolated nucleic acid molecule of claim 1, wherein the substitution at encoded amino acid 214 comprises a glutamic acid for a lysine (E214K) and wherein the substitution at encoded amino acid 223 comprises a glutamic acid for a lysine (E223K).

4. The isolated nucleic acid molecule of claim 1, wherein the encoded designer Cx34.7 connexin protein comprises the sequence set forth in SEQ ID NO:19 with a substitution at encoded amino acid 214 and at encoded amino acid 223.

5. The isolated nucleic acid molecule of claim 1, wherein the nucleic acid sequence encoding the designer Cx34.7 connexin protein comprises the sequence set forth in SEQ ID NO:23 or SEQ ID NO:24 or a fragment thereof.

6.-7. (canceled)

8. An isolated nucleic acid molecule, comprising: a nucleic acid sequence encoding a designer Cx35 connexin protein, wherein the designer Cx35 connexin protein comprises the Cx35 of Morone americana having a substitution at encoded amino acid 221.

9. (canceled)

10. The isolated nucleic acid molecule of claim 8, wherein the substitution at encoded amino acid 221 comprises a lysine for a glutamic acid (K221E).

11. The isolated nucleic acid molecule of claim 8, wherein the encoded designer Cx35 connexin protein comprises the sequence set forth in SEQ ID NO:20 with a substitution at encoded amino acid 221.

12. The isolated nucleic acid molecule of claim 8, wherein the nucleic acid sequence encoding the designer Cx35 connexin protein comprises the sequence set forth in SEQ ID NO:25 or SEQ ID NO:26 or a fragment thereof.

13.-14. (canceled)

15. A viral vector, comprising: the isolated nucleic acid molecule of claim 1.

16. A viral vector, comprising: the isolated nucleic acid molecule of claim 8.

17.-36. (canceled)

37. A method of precision editing a neural circuit, the method comprising:
generating a synthetic gap junction between a targeted first type of cells and targeted second type of cells in the central nervous system in a subject in need thereof,
wherein the synthetic gap junction facilitates electrical activity between the first type of cells and the second type of cells.

38. The method of claim 37, wherein the synthetic gap junction exhibits rectification in the Cx34.7 to Cx35 direction.

39. The method of claim 37, wherein generating a synthetic gap junction comprises:
administering to a subject a first viral vector targeting the first type of cells; and
administering to the subject a second viral vector targeting the second type of cells,
wherein the first type of cells is different than the second type of cells.

40. The method of claim 39, wherein the first viral vector comprises the vector of claim 15.

41. The method of claim 39, wherein the second viral vector comprises the vector of claim 15.

42. The method of claim 40, wherein following expression of the designer connexin protein, the first type of cells comprises a hemichannel comprising Cx34.7_{E214K/E223K}.

43. The method of claim 41, wherein following expression of the designer connexin protein, the second type of cells comprises a hemichannel comprising Cx35_{K221E}.

44. (canceled)

45. The method of claim 39, wherein the targeted first type of cells comprise pre-synaptic neurons and wherein the targeted second type of cells comprises post-synaptic neurons.

46. (canceled)

47. The method of claim 37, wherein the subject in need thereof has a neurological disease and/or disorder and/or wherein the subject in need thereof has a psychiatric disease and/or disorder.

48. The method of claim 37, wherein the subject's behavior is modulated and/or wherein the subject's physiology is modulated.

49.-65. (canceled)
